(19) United States
(12) Patent Application Publication
(10) Pub. No.: US 2025/0262454 A1
(43) Pub. Date: Aug. 21, 2025

(54) METHODS AND DEVICES FOR PHOTOBIOMODULATION

(71) Applicant: JelikaLite LLC, New York, NY (US)

(72) Inventors: Eugenia Steingold, New York, NY (US); Katya Sverdlov, New York, NY (US); Kameshwar Durvasula, Fair Lawn, NJ (US)

(21) Appl. No.: 19/197,619

(22) Filed: May 2, 2025

Related U.S. Application Data
(63) Continuation of application No. PCT/US23/36721, filed on Nov. 2, 2023.
Application No. PCT/US23/36721 is a continuation-in-part of application No. 17/949,997, filed on Sep. 21, 2022.
(63) Continuation-in-part of application No. 17/949,997, filed on Sep. 21, 2022.
(60) Provisional application No. 63/541,995, filed on Oct. 2, 2023.
(60) Provisional application No. 63/429,815, filed on Dec. 2, 2022.
(60) Provisional application No. 63/421,887, filed on Nov. 2, 2022.

(51) Int. Cl. A61N 5/06

(52) U.S. Cl. CPC A61N 5/0622; A61N 2005/0626; A61N 2005/0647; A61N 2005/0652; A61N 2005/0659; A61N 2005/0663

(57) ABSTRACT

Systems and methods are described for treatment of neurological conditions in which transcranial illumination using infrared, near-infrared and/or red wavelengths of light are delivered into the brain of a patient using a portable head wearable device. Systems and methods are also described to deliver light to patient tissues for photobiomodulation, particularly through the patient's mouth.

### CROSS REFERENCE TO RELATED APPLICATIONS

This application is a continuation application of International Application No. PCT/US2023/036721 filed on Nov. 2, 2023, which is a continuation in part of U.S. patent application Ser. No. 17/949,997 filed Sep. 21, 2022 and which claims the benefit of and priority to U.S. Provisional Application No. 63/421,887 filed on Nov. 2, 2022, U.S. Provisional Application No. 63/429,815 filed Dec. 2, 2022 and U.S. Provisional Application No. 63/541,995, filed Oct. 2, 2023. This application is also a continuation in part of U.S. patent application Ser. No. 17/949,997 filed Sep. 21, 2022, the entire contents of each of the above-mentioned applications being incorporated herein by reference in their entirety.

### FIELD OF THE INVENTION

The presently disclosed subject matter relates generally to methods and devices for transcranial illumination for the therapeutic treatment of neurological conditions. Preferred embodiments can include wearable devices that communicate with mobile devices such as web enabled phones and tablets to facilitate system operation and patient data analysis. This can optionally include cross-modal brain stimulation, diagnostic modalities and, more particularly, provide methods and devices for treating children suffering from autism that can optionally utilize simultaneous audio and light stimulation.

### BACKGROUND

Research indicates that in treating many neurological and psychiatric conditions, a strong combinatory effect of two separate types of treatments exists. For example, in the treatment of depression and anxiety, a combination of both medications and cognitive behavioral therapy (or dialectic behavioral therapy) produces stronger effects than either one of those modalities independently.

Furthermore, music therapy and videos games have been used to treat epilepsy patients. Some of the results indicate that listening to specific musical content in combination with pharmacological treatment reduced both the frequencies of epileptic discharges and frequencies of seizures. Similarly, combining video games with pharmacological treatment has also been shown to modulate the brain neuroplasticity and improve age-related neuronal deficits and enhanced cognitive functions in older adults. Therefore, adding two types of different treatments together has been shown to improve the outcome of the overall treatment of neurological and psychiatric conditions across various domains. Overall, when treating psychiatric or neurological disorder, combinatory effects of brain stimulation through various channels is likely to be stronger than unimodal stimulation.

For children diagnosed with Autism Spectrum Disorder (“ASD”), one of the most common challenges they face is learning language. Studies show that children with ASD struggle with acquiring syntax. As a result, they cannot parse sentences, understand speech, and/or acquire or produce new words. In particular, learning language by the age of five (being able to speak full sentences) is critical for future successful integration with neuro-typical community and independent functioning. In addition, language learning may only occur during the sensitive period (REFS), which ends between 5-7 years of age. If a child does not fully learn language during that period, subsequent learning is highly effortful and achieving fluency is unlikely. Furthermore, being able to comprehend and produce language reduces tantrums and improves behavior in individuals with ASD. Therefore, delays in speech development is one of the most critical symptoms that needs to be alleviated.

Another critical symptom that needs to be alleviated in children with ASD is anxiety. General anxiety is frequently quite debilitating in ASD children and it affects, among other things, children's ability to learn and ability to integrate socially. Children with ASD are frequently prescribed medication to reduce their anxiety, but these medications often have unintended side effects and may be not effective.

In the United States, there are over 1.5 MM children currently diagnosed with ASD, and approximately 80,000 new children are diagnosed with ASD annually. Across the world, approximately 1.5 MM-2 MM new children are annually diagnosed with ASD. Autism services cost Americans approximately $250 billion a year, which includes both medical costs (outpatient care, home care, and drugs) and non-medical costs (special education services, residential services, etc.). In addition to outright costs, there are hidden ones, such as emotional stress as well as the time required to figure out and coordinate care. Research indicates that lifelong care costs can be reduced by almost two thirds with proper early intervention. Further research indicates that ASD is often correlated with mitochondrial dysfunction. Mitochondria in brain cells of autistic individuals does not produce enough adenosine triphosphate (“ATP”). The result of mitochondria dysfunction may be especially pronounced in the brain, since it uses 20% of all the energy generated by the human body, which may lead to neuro-developmental disorders, such as ASD. Encouraging research has shown that infrared and red light may activate a child's mitochondria, and therefore increase ATP production. The metabolic pathways impacting neurological function have been studied in significant detail. See, for example, Naviaux, “Metabolic features and regulation of the healing cycle-A new model for chronic disease pathogenesis and treatment”, Mitochondrion. 2019 May; 46:278-297. doi: 10.1016/j.mito.2018.08.001. Epub 2018 Aug. 9. Note also, Mason et al. “Nitric Oxide inhibition of respiration involves both competitive (heme) and noncompetitive (copper) binding to cytochrome c oxidase”, PNAS, vol. 103, no. 3, Jan. 17, 2006, the entire contents of the above two publications being incorporated herein by reference.

Transcranial photobiomodulation (“tPBM”) of the brain with near infrared and red light has been shown to be beneficial for treating various psychiatric and neurological conditions such as anxiety, stroke and traumatic brain injury. Remarkably, autism spectrum disorder may potentially be treated therapeutically with tPBM as several scientists have recently linked the disorder to mitochondria disbalance and tPBM can potentially affect mitochondria by causing it to produce more ATP. Patients treated with tPBM will absorb near infrared light, which can potentially reduce inflammation, increase oxygen flow to the brain and increase production of ATP. However, devices and methods are needed that will enable additional treatment options for various neurological conditions.

One problem with language acquisition is that many children with ASD cannot focus on the language enough to extract syntactic features of words, to parse sentences, and/or to attend to syntactic and semantic clues of speech. Therefore, their word learning may be delayed.

The problem with anxiety is that ASD children frequently get very stressed and do not know how to calm themselves before a particular learning or social situation. As a result, they are unable to participate in regular activities (such as playdates or classes).

Accordingly, there is a need for improved methods and devices providing treatment of neurological disorders and to specifically provide therapies for the treatment of children.

### SUMMARY

Preferred embodiments provide devices and methods in which a head wearable device is configured to be worn by a subject that is operated to deliver illuminating wavelengths of light with sufficient energy that are absorbed by a region of brain tissue during a therapeutic period. Transcranial delivery of illuminating light can be performed with a plurality of light emitting devices mounted to the head wearable device that can also preferably include control and processing circuitry. Therefore, providing brain stimulation with one or more of, or combinations of (i) infrared, near infrared and red light to improve operational states of the brain such as by ATP production in the brain, for example, and (ii) provide additional specific linguistic input(s) to learn syntax will improve language acquisition in ASD children. Therefore, providing brain stimulation with a combination of (i) near infrared and red light to reduce anxiety and (ii) specific meditations written for ASD children will reduce anxiety. Reduced anxiety leads to both improved language learning and better social integration. Providing an audio language program specifically designed for ASD children, may focus the attention of the child on the language, provide the child with the information about linguistic markers, and improve the child's ability to communicate. This is likely to reduce lifelong care costs for affected individuals.

Preferred embodiments can use a plurality of laser diodes or light emitting diodes (LEDs) configured to emit sufficient power through the cranium of a patient to provide a therapeutic dose during a therapeutic period. This plurality of light emitting devices can be mounted to circuit boards situated on a head wearable device. For the treatment of children the spacing between light emitters in each array mounted to the head wearable device can be selected to improve penetration depth through the cranium. As the cranium of a child increases in thickness with age, the parameters of light used to penetrate the cranium will change as a function of age. As attenuation of the illuminating light will increase with age, the frequency of light, power density and spot size of each light emitter can be selectively adjusted as a function of age. The system can automatically set the illumination conditions as a function of age of the patient. The thickness of the cranium of an individual patient can also be quantitatively measured by x-ray scan and entered into the system to set the desired illumination parameters needed to deliver the required power density to the selected region of the brain. The density of the cranium can also change as a function of age and can be quantitatively measured by x-ray bone densitometer to generate further data that can be used to control and adjust the level of radiance applied to different regions of the cranium.

Aspects of the disclosed technology include methods and devices for cross-modal stimulation brain stimulation, which may be used to treat ASD children. Consistent with the disclosed embodiments, the systems and methods of their use may include a wearable device (e.g., a bandana) that includes one or more processors, transceivers, microphones, headphones, LED lights (diodes), or power sources (e.g., batteries). One exemplary method may include positioning the wearable device on the head of a patient (an ASD child). The method may further include transmitting, by the wearable device (e.g., the LED lights), a pre-defined amount of light (e.g., red or near infrared light). The method may also include simultaneously outputting, by the headphones of the wearable device or other device that can be heard or seen by the patient, a linguistic input to the patient, for example. The linguistic input may include transparent syntactic structures that facilitate, for example, learning how to parse sentences. Also, the method may include outputting specific meditations written for ASD children, that may help case anxiety, and thus allowing ASD children to better learn language and more easily integrate socially. In some examples, the method may further include receiving a response to the linguistic input from the patient, that the one or more processors may analyze to determine the accuracy of the response and/or to generate any follow-up linguistic inputs. Further, in some examples, the frequency and/or type of light outputted by the wearable device may be adjusted based on the response received from the patient. Also, in some examples, the wearable device may be paired to a user device (e.g., via Bluetooth®) that determines and sends the linguistic input(s) to the wearable device or other devices including one or more transducer devices, such as speakers, or display devices that can generate auditory or visual signals/images that can be heard and/or seen by the patient.

The battery powered headset can preferably be configured with an onboard power control device that automatically controls optical power output of the device during a therapeutic session. Therapeutic sessions can have preset operating conditions for each patient, or a class of patients, as described herein whereby a power distribution circuit board can independently control current levels through each of a plurality of light sources at a selected frequency and duty cycle. Preferred embodiments provide closed loop control of each light source such that the emitted light signal remains within 10% of a nominal value, and preferably within 5% of the selected nominal value. Safety features can be implemented in preferred embodiments in which a sensor can be used to monitor a selected operating condition of the head mounted device. For example, if a patient alters the position of or removes the headset form his or her head during a session, the sensor can transmit a signal to the system control which can, depending upon the received signal, switch off the power to the headset and/or record the time of the signal reception, and optionally send a signal to a remote device by wireless or wired connection communicating the change in state of the device. In another example, if light being emitted by one of the light sources exceeds a threshold value, or if an operating temperature of a component in the optoelectrical system exceeds a threshold temperature, this will trigger a shutoff of the light sources and cause a signal to be sent to an external device communicating the change in operating condition and record time and cause of the change. As the length of a therapeutic session may vary from patient to patient, the operating conditions can be selected based on a plurality of preset operating parameters stored in a device memory. A therapeutic session may last for at least 5 minutes for treatment of certain conditions, whereas the session may last at least 10 minutes for a further condition, and may last 15 or more minutes for a further distinct condition. Power levels may vary for each of these different treatment modules and different light sources may be controlled differently during one or more sessions.

The head wearable device can comprise rigid, semi-rigid or flexible substrates on which the light emitters and circuit elements are attached. The flexible substrates can include woven fabrics or polymer fibers, molded plastics or machine printed components assembled into a band that extends around the head of the patient. Circuit boards on which electrical and optical components are mounted and interconnected can be standard rigid form or they can be flexible so as to accommodate bending around the curvature of the patient's head. As children and adults have heads in a range of different sizes, it is advantageous to have a conformable material that can adjust to different sizes. More rigid head wearable devices can use foam material to provide a conformable material in contact with the patient's head. The head wearable device can be used in conjunction with diagnostic devices and systems that can be used to select the parameters for the therapeutic use of light as described herein. A computing device such as a tablet or laptop computer can be used to control diagnostic and therapeutic operations of the head worn device and other devices used in conjunction with a therapeutic session. Such computing devices can store and manage patient data and generate electronic health or medical records for storage and further use. The computing device can be programmed with software modules such as a patient data entry module, a system operating module that can include diagnostic and therapeutic submodules, and an electronic medical records module. The system can include a networked server to enable communication with remote devices, web/internet operations and remote monitoring and control by secure communication links. The computing device can include connections to electroencephalogram (EEG) electrodes to monitor brain activity before, during or after therapeutic sessions to generate diagnostic data for the patient. The EEG electrodes can be integrated with the head wearable device and be connected either directly to a processor thereon, or alternatively, can communicate by wired or wireless connection to the external computing device such as a touchscreen operated tablet display device. Light sensors that are optically coupled to the head of the patient can be used to monitor light delivery into the cranium of the patient and/or can measure light returning from the regions of the brain that receive the illuminating light. An array of near infrared sensors can be mounted on the LED panels or circuit boards, for example, that can detect reflected light or other light signals returning from the tissue that can be used to diagnose a condition of the tissue. Diagnostic data generated by the system sensors can be used to monitor the patient during a therapeutic period and can optionally be used to control operating parameters of the system during the therapy session such as by increasing or decreasing the intensity of the light delivered through the cranium or adjusting the time period or areas of the brain being illuminated during the therapy session.

For the treatment of children having an autism spectrum disorder, they often are not responsive to instructions, may exhibit behaviors such as self-injury or attempt to injure others, and may exhibit movements that are not conducive to standard therapeutic treatment. Specifically, it can be necessary with many patients that a device placed on the head must be light in weight and be untethered such as by a wired connection during treatment. Consequently, it is important to have a battery powered device that does not have a wired connection during a therapeutic period or session. Any communication that occurs between the head mounted device and an external device used to control and/or monitor the device during a therapeutic period is preferably performed by wireless connection. Thus, an external computing device such as a mobile communication device such as a mobile phone or a tablet display device can communicate wirelessly with the head mounted device. Such devices can include one or more processors configured to stream data to and from the head mounted device. Such devices are connectable to private or public communications networks to facilitate communication with parents and teachers, for example, that are involved with a child's treatment, medical history and education plan.

Machine learning tools can be employed to process data generated by the devices and methods described herein. Methods such as principal component analysis (PCA), support vector machines (SVM), convolutional and/or recurrent neural networks, clustering and other numeric and quantitative methods can be employed to characterize therapeutic outcomes and generate operational parameters for different classes of patients that exhibit different behavioral and/or medical conditions that can be effectively treated by photobiomodulation therapy. Neurologic conditions can impact sleep patterns and learning capacity of children and such computational methods can be used to improve therapeutic treatment.

Further features of the disclosed design, and the advantages offered thereby, are explained in greater detail hereinafter with reference to specific embodiments illustrated in the accompanying drawings, wherein like elements are indicated be like reference designators.

### BRIEF DESCRIPTION OF THE DRAWINGS

Reference will now be made to the accompanying drawings, which are not necessarily drawn to scale, are incorporated into and constitute a portion of this disclosure, illustrate various implementations and aspects of the disclosed technology, and, together with the description, serve to explain the principles of the disclosed technology. In the drawings:

FIG. 1 is an example head wearable device, in accordance with some examples of the present disclosure.

FIGS. 2A-2C show rear, side and front views of a patient with the head wearable device of FIG. 1.

FIG. 3 illustrates use of a portable phone or tablet device connected to the head wearable device.

FIG. 4 schematically illustrates the operating elements of the head wearable device and control features.

FIG. 5 schematically illustrates the components of a head wearable device in accordance with preferred embodiments.

FIG. 6 illustrates a screen shot of a testing procedure used with preferred embodiments of the invention.

FIG. 7 is a process flow diagram in accordance with preferred methods of operating the head wearable device and control system.

FIG. 8 is a process flow diagram illustrating the use of EEG measurements in conjunction with transcranial illumination of a patient.

FIG. 9 illustrates a table with exemplary parameters having variable ranges between upper and lower thresholds used for transcranial illumination of a patient in accordance with preferred embodiments.

FIG. 10 illustrates a process flow diagram for selecting and optimizing parameters over multiple therapeutic sessions including manual and automated selection tracks.

FIG. 11 illustrates a process flow diagram for administering a therapeutic session to a patient in accordance with various embodiments described herein.

FIG. 12A illustrates a further view of a head worn device having circuit housing elements accessible to a user that is communicably connected to a first tablet device used by the patient during a therapy session and a second tablet used by an operator to monitor, control and/or program the system for diagnostic and therapeutic use as described generally herein.

FIG. 12B illustrates a top view of a head mounted photobiomodulation system including an electroencephalographic (EEG) electrode system with wireless transmission of data to an external processing system.

FIG. 12C shows a rear view of the system of FIG. 12B with light emitting and/or sensor arrays mounted on a rear band configured for positioning to transmit and/or receive signals through the cranium via selected transmission paths such as, for example, the lambdoid suture and/or the squamosal suture.

FIG. 12D illustrates an enlarged side cross-sectional view of a spring mounted light emitting array, a sensor array, or a combination thereof in accordance with embodiments described herein.

FIG. 12E illustrates a side view of a head wearable device in accordance with some embodiments described herein.

FIG. 12F illustrates a rear view of the head wearable device of FIG. 12E.

FIG. 12G illustrates an alternative embodiment of the head wearable device with different placement of the head strap in accordance with some embodiments described herein.

FIG. 12H illustrates a head wearable device according to various embodiments described herein.

FIG. 12I illustrates placement of an LED module in a core of a headband in accordance with some embodiments described herein.

FIG. 12J illustrates placement of the LED module in a completed headband in accordance with some embodiments described herein.

FIG. 12K schematically illustrates the electrical connections between elements of the head wearable device in accordance with several embodiments described herein.

FIGS. 12L and 12M illustrate top and bottom views, respectively, of a power printed circuit board in accordance with some embodiments described herein.

FIGS. 12N and 12O illustrate top and bottom views, respectively, of an occipital power distribution printed circuit board in accordance with some embodiments described herein.

FIGS. 12P and 12Q illustrate top and bottom views, respectively, of a frontal power distribution printed circuit board in accordance with some embodiments described herein.

FIGS. 12R and 12S illustrate top and bottom views, respectively, of an LED printed circuit board in accordance with some embodiments described herein.

FIG. 13A illustrates a further view of a head worn device having circuit housing elements accessible to a user that is communicably connected to a first tablet device used by the patient during a therapy session and a second tablet used by an operator to monitor, control and/or program the system for diagnostic and therapeutic use as described generally herein.

FIG. 13B illustrates a rear view of a head mounted photobiomodulation system including an electroencephalographic (EEG) electrode system with wireless transmission of data to an external processing system.

FIG. 13C shows an exploded view of the system of FIG. 13B with light emitting and/or sensor arrays mounted on a rear band configured for positioning to transmit and/or receive signals through the cranium via selected transmission paths such as, for example, the lambdoid suture and/or the squamosal suture.

FIG. 13D shows a rear exploded view of the occipital mount and its housing.

FIG. 13E shows a front exploded view of the system of FIG. 13D. illustrates a side view of a head wearable device in accordance with some embodiments described herein.

FIG. 13F illustrates a portion of the occipital mount that rests against the patient's cranium.

FIG. 13G illustrates a head wearable device according to various embodiments described herein.

FIG. 13H illustrates placement of LED modules throughout a headband in accordance with some embodiments described herein.

FIG. 13I illustrates placement of racetracks throughout a headband to allow positioning of LED modules.

FIG. 13J illustrates an LED in a racetrack and translating.

FIG. 13K illustrates the layers of a headband.

FIG. 13L shows a headband adjusting in length.

FIG. 13M illustrates a side view of a LED module.

FIG. 13N shows an interior view of a LED module housing.

FIG. 13O illustrates an exploded view of a LED module with a LED panel.

FIG. 13P illustrates a side view of an alternative embodiment of a LED module.

FIG. 13Q illustrates a front view of the LED module of FIG. 13P.

FIG. 13R illustrates an internal view of the LED module of FIG. 13P.

FIG. 13S illustrates LED modules mounted to the occipital mount.

FIG. 13T illustrates exemplary LED circuit board details for a first side of the circuit board on which the LED is mounted.

FIG. 13U illustrates exemplary LED circuit board details for a second side of the circuit board on which the microcontroller is mounted.

FIG. 13V illustrates patient ergonomics and various head strap sizes in various embodiments taught herein

FIG. 13W illustrates an exemplary control panel in various embodiments taught herein.

FIG. 13X illustrates exemplary connector pod locations in various embodiments taught herein.

FIG. 13Y illustrates exemplary wiring channels in various embodiment taught herein.

FIG. 13Z illustrates exemplary rear LED PCB wiring in various embodiments taught herein.

FIG. 13AA illustrates exemplary capacitive touch sensor locations.

FIG. 13AB illustrates exemplary add-on LED locations in a housing in various embodiments taught herein.

FIG. 14 illustrates a process sequence that can be implemented with the therapeutic devices described herein.

FIG. 15 illustrates a circuit for operating photobiomodulation devices of the present description.

FIG. 16 shows resting power as a function of frequency bands for patients with different diagnoses.

FIG. 17A is a block diagram representing the user assessment, personalized treatment selection, and performance feedback process.

FIG. 17B is an illustration of regions of the brain that can be selected for treatment in various embodiments taught herein.

FIG. 18 illustrates a method for therapeutic photobiomodulation for treatment of diseases or disorders in accordance with some embodiments described herein.

FIG. 19 illustrates a graphical user interface including user prompts to resolve patient status requirements accordance with various embodiments taught herein.

FIG. 20 illustrates a flowchart for a method for therapeutic photobiomodulation for treatment of diseases or disorders in accordance with various embodiments taught herein.

### DETAILED DESCRIPTION

Some implementations of the disclosed technology will be described more fully with reference to the accompanying drawings. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

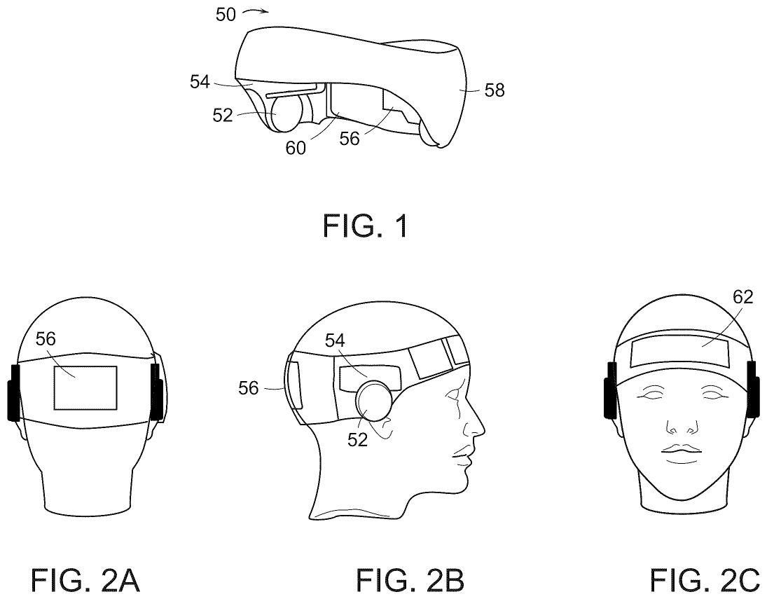

FIG. 1 shows an example wearable device 50 that may implement certain methods for cross-modal brain stimulation. As shown in FIG. 1, in some implementations the wearable device 50 may include one or more processors, transceivers, microphones, headphones 52, LED lights 54, and/or batteries, amongst other things. The wearable device 50 may be paired with a user device (e.g., smartphone, smartwatch), which may provide instructions that may determine a frequency of transmitted light, the type of light (e.g., red light or infrared light), the meditations, and/or the linguistic inputs. FIGS. 2A-2C depict rear side and front views of the head wearable device 50 positioned on the head of a patient with rear circuit board 56, side illumination panels 56, and front illumination panel 62 to provide transcranial illumination, and also earphones 52 to provide audio programming to the patient. The system can store audio files or video files that can be heard or seen by the user in conjunction with the therapeutic session for a patient.

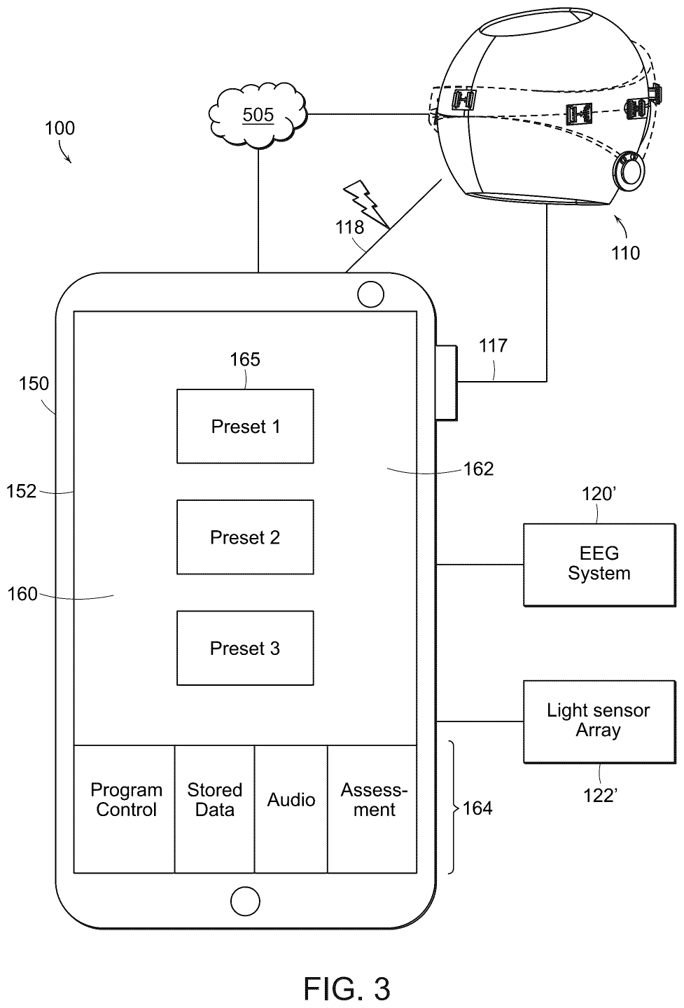

FIG. 3 is an illustration of a system 100 for brain stimulation in accordance with various embodiments described herein. The system 100 includes a photobiomodulation device 110 in communication with a remote computing device 150. In exemplary embodiments, the computing device 150 includes a visual display device 152 that can display a graphical user interface (GUI) 160. The GUI 160 includes an information display area 162 and user-actuatable controls 164. Optionally, the computing device 150 is also in communication with an external EEG system 120′. Optionally, the computing device 150 is also in communication with an external light sensor array 122′. An operating user can operate the computing device 150 to control operation of the photobiomodulation device 110 including activation of the functions of the photobiomodulation device 110 and mono- or bi-directional data transfer between the computing device 150 and the photobiomodulation device 110. Further details concerning devices and methods for performing photobiomodulation to diagnose and treat neurological disorders can be found in U.S. patent application Ser. No. 17/949,997, filed Sep. 21, 2022, which is a continuation-in-part of International Application No. PCT/US2022/020770, filed Mar. 17, 2022, which claims priority to U.S. Provisional Application No. 63/303,384, filed Jan. 26, 2022, and to U.S. Provisional Application No. 63/272,823, filed Oct. 28, 2021, and to U.S. Provisional Application No. 63/250,703, filed Sep. 30, 2021, and to U.S. Provisional Application No. 63/162,484, filed Mar. 17, 2021, the entire contents of each of these applications being incorporated herein by reference.

The operating user can change among operational modes of the computing device 150 by interacting with the user-actuatable controls 164 of the GUI 160. Examples of user-actuatable controls include controls to access program control tools, stored data and/or stored data manipulation and visualization tools, audio program tools, assessment tools, and any other suitable control modes or tools known to one of ordinary skill in the art. Upon activation of the program control mode, the GUI 160 displays program control information in the information display area 162. Likewise, activation of other modes using user-actuatable controls 164 can cause the GUI 160 to display relevant mode information in the information display area 162. The system can be programmed to perform therapeutic sessions with variable lengths of between 5 and 30 minutes, for example. The patient's use of language during the session can be recorded by microphone on the head wearable device or used separately and an analysis of language used during the session or stored for later analysis.

In the program control mode, the GUI 160 can display program controls including one or more presets 165. Activation of the preset by the operating user configures the photobiomodulation device 110 to use specific pre-set variables appropriate to light therapy for a particular class of patients or to a specific patient. For example, a specific preset 165 can correspond to a class of patient having a particular age or particular condition. In various embodiments, the pre-set variables that are configured through the preset 165 can include illumination patterns (e.g., spatial patterns, temporal patterns, or both spatial and temporal patterns), illumination wavelengths/frequencies, or illumination power levels.

In some embodiments, the photobiomodulation device 110 can transmit and/or receive data from the computing device 150. For example, the photobiomodulation device 110 can transmit data to log information about a therapy session for a patient. Such data can include, for example, illumination patterns, total length of time, time spent in different phases of a therapy program, electroencephalogram (EEG) readings, and power levels used. The data can be transmitted and logged before, during, and after a therapy session. Similar data can also be received at the computing device 150 from the external EEG system 120′ or the external light sensor array 122′ in embodiments that utilize these components. In the stored data manipulation and/or visualization mode, the operating user can review the data logged from these sources and received at the computing device 150. In some embodiments, the data can include information regarding activities used in conjunction with the therapy session (i.e., information related to tasks presented to the patient during the therapy session such as task identity and scoring). For example, activity data can be input by an operating user on the assessment mode screen as described in greater detail below.

In the audio system mode, the user can control audio information to be delivered to the patient through speakers 116 of the photobiomodulation device 110. Audio information can include instructions to the patient in some embodiments. In other embodiments, audio information can include audio programming for different therapeutic applications.

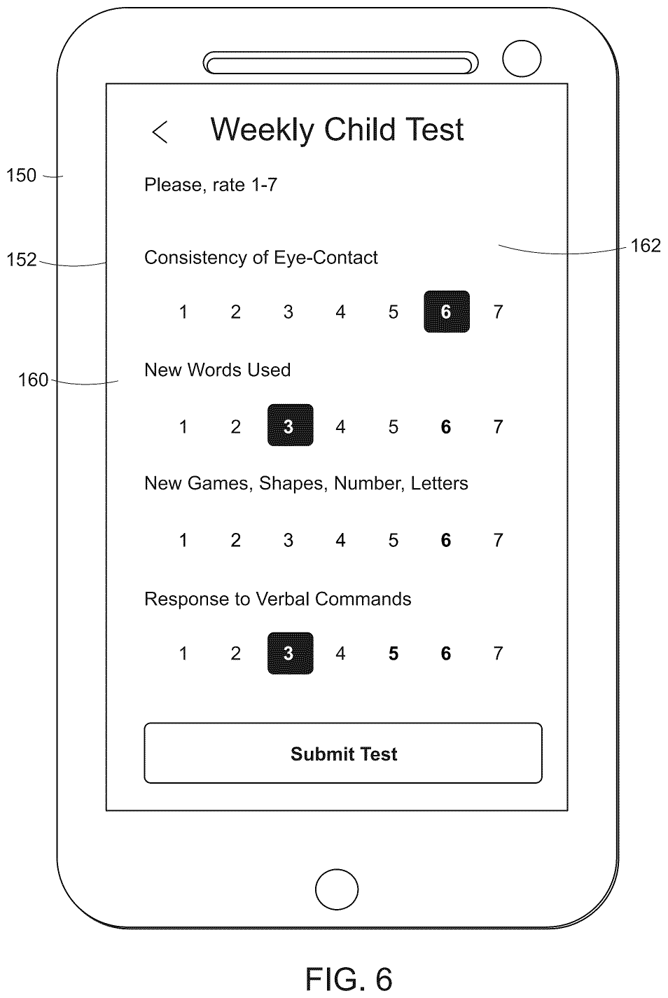

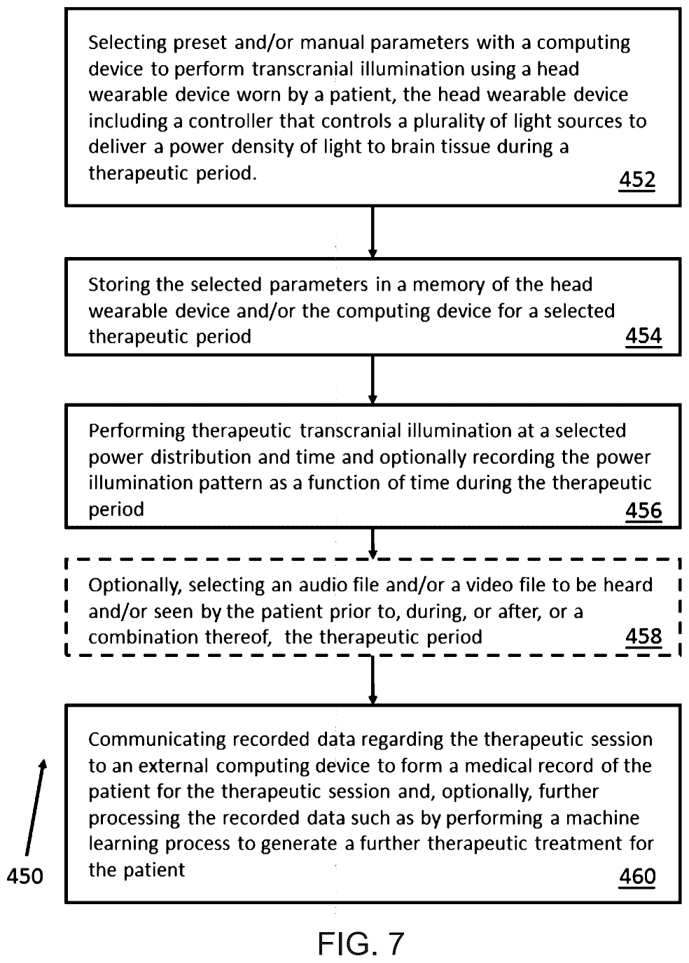

In the assessment mode, a user can input or review data related to patient assessment such as task identity and scoring. For example, FIG. 6 illustrates a particular assessment test displayed in the information display area 162 of the GUI 160. This assessment test, the Weekly Child Test, includes rating scales representing scoring on a variety of individual metrics geared to an overall assessment of the severity of autism in the child.

As described in greater detail below, the computing device 150 and photobiomodulation device 110 can communicate through a variety of methods. In some embodiments, a direct (i.e., wired) connection 117 can be established between the computing device 150 and the photobiomodulation device 110. In some embodiments, the computing device 150 and the photobiomodulation device 110 can communicate directly with one another through a wireless connection 118. In still further embodiments, the computing device 150 and the photobiomodulation device 110 can communication through a communications network 505.

In various embodiments, one or more portions of the communications network 505 can be an ad hoc network, a mesh network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a Wi-Fi network, a WiMAX network, an Internet-of-Things (IoT) network established using Bluetooth® or any other protocol, any other type of network, or a combination of two or more such networks.

In exemplary embodiments, the system 100 is configured to treat autistic patients and, in particular, juvenile autistic patients. As such, it is desirable in many embodiments to create a wireless connection between the photobiomodulation device 110 and the computing device 150 as a juvenile patient is less likely to sit still for the length of a therapy session. Wireless connection and use of a battery to power the photobiomodulation device 110 enables uninterrupted transcranial illumination for the entire length of a single therapy session and, further, enables the juvenile patient to move and engage in activities that may, or may not, be associated with the therapy.

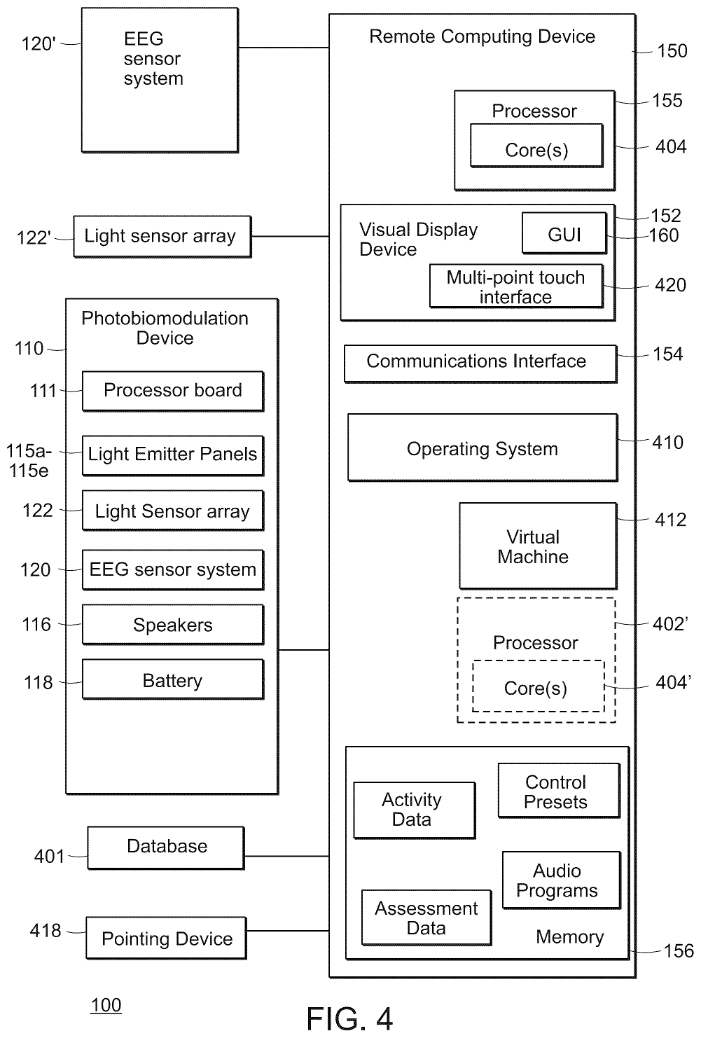

FIG. 4 shows block diagrams of a remote computing device 150 and photobiomodulation device 110 suitable for use with exemplary embodiments of the present disclosure. The remote computing device 150 may be, but is not limited to, a smartphone, laptop, tablet, desktop computer, server, or network appliance. The remote computing device 150 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 156 included in the remote computing device 150 may store computer-readable and computer-executable instructions or software for implementing exemplary operations of the remote computing device 150. The remote computing device 150 also includes configurable and/or programmable processor 155 and associated core(s) 404, and optionally, one or more additional configurable and/or programmable processor(s) 402′ and associated core(s) 404′ (for example, in the case of computer systems having multiple processors/cores), for executing computer-readable and computer-executable instructions or software stored in the memory 156 and other programs for implementing exemplary embodiments of the present disclosure. Processor 155 and processor(s) 402′ may each be a single core processor or multiple core (404 and 404′) processor. Either or both of processor 155 and processor(s) 402′ may be configured to execute one or more of the instructions described in connection with remote computing device 150.

Virtualization may be employed in the remote computing device 150 so that infrastructure and resources in the remote computing device 150 may be shared dynamically. A virtual machine 412 may be provided to handle a process running on multiple processors so that the process appears to be using only one computing resource rather than multiple computing resources. Multiple virtual machines may also be used with one processor.

Memory 156 may include a computer system memory or random access memory, such as DRAM, SRAM, EDO RAM, and the like. Memory 156 may include other types of memory as well, or combinations thereof.

A user may interact with the remote computing device 150 through a visual display device 152, such as a computer monitor, which may display one or more graphical user interfaces 160. In exemplary embodiments, the visual display device includes a multi-point touch interface 420 (e.g., touchscreen) that can receive tactile input from an operating user. The operating user may interact with the remote computing device 150 using the multi-point touch interface 420 or a pointing device 418.

The remote computing device 150 may also interact with one or more computer storage devices or databases 401, such as a hard-drive, CD-ROM, or other computer readable media, for storing data and computer-readable instructions and/or software that implement exemplary embodiments of the present disclosure (e.g., applications). For example, exemplary storage device 401 can include modules to execute aspects of the GUI 160 or control presets, audio programs, activity data, or assessment data. The database(s) 401 may be updated manually or automatically at any suitable time to add, delete, and/or update one or more data items in the databases. The remote computing device 150 can send data to or receive data from the database 401 including, for example, patient data, program data, or computer-executable instructions.

The remote computing device 150 can include a communications interface 154 configured to interface via one or more network devices with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections (for example, WiFi or Bluetooth®), controller area network (CAN), or some combination of any or all of the above. In exemplary embodiments, the remote computing device 150 can include one or more antennas to facilitate wireless communication (e.g., via the network interface) between the remote computing device 150 and a network and/or between the remote computing device 150 and the photobiomodulation device 100. The communications interface 154 may include a built-in network adapter, network interface card, PCMCIA network card, card bus network adapter, wireless network adapter, USB network adapter, modem or any other device suitable for interfacing the remote computing device 150 to any type of network capable of communication and performing the operations described herein.

The remote computing device 150 may run operating system 410, such as versions of the Microsoft® Windows® operating systems, different releases of the Unix and Linux operating systems, versions of the MacOS® for Macintosh computers, embedded operating systems, real-time operating systems, open source operating systems, proprietary operating systems, or other operating system capable of running on the remote computing device 150 and performing the operations described herein. In exemplary embodiments, the operating system 410 may be run in native mode or emulated mode. In an exemplary embodiment, the operating system 410 may be run on one or more cloud machine instances.

The photobiomodulation device 110 can include a processor board 111, one or more light emitter panels 115a-115e, one or more speakers 116, and one or more batteries 118. The photobiomodulation device 110 can optionally include a light sensor array 122 and an EEG sensor system 120. Although five light emitter panels 115a-115e are described with respect to this disclosure, one or ordinary skill in the art would appreciate that a greater or fewer number of panels may be used. In an exemplary embodiment, the light emitter panels 115a-115e hare flexible. In an exemplary embodiment, the light emitter panels 115a-115e are positioned at the front, top, back, and both sides of the user's head. In embodiments wherein the photobiomodulation device 110 does not have a full cap over the user's head (i.e., a headband-style device), the top panel may be omitted.

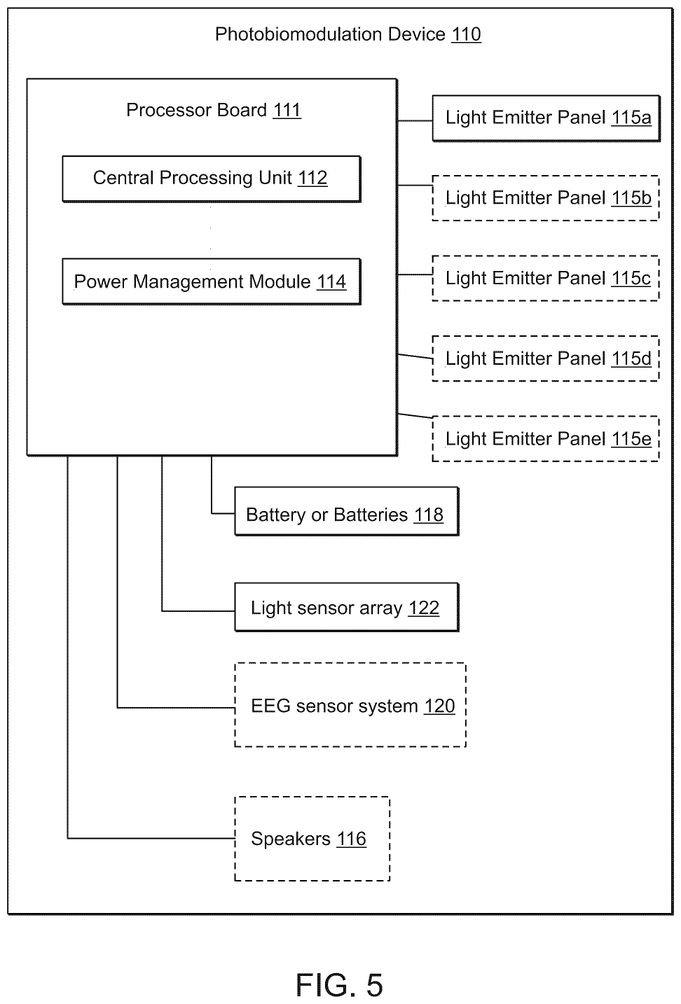

FIG. 5 illustrates a schematic layout of the photobiomodulation device 110 of the present invention. The processor board 111 is, for example, a printed circuit board including components to control functions of the photobiomodulation device 110. The processor board 111 can include a central processing unit 112 and a power management module 114 in some embodiments.

The power management module 114 can monitor and control use of particular light emitter panels 115a-115e during a therapy session. In some embodiments, the power management module 114 can take action to control or provide feedback to a patient user related to whether light emitter panels 115a-115e are not used, or are only partially used, during a particular therapy session. By mitigating use of certain panels during a session, longer operation can be achieved. Moreover, different classes of patient (e.g., patients of different ages) can have different cranial thicknesses. As a result, different transmission power (and penetration) may be necessary as a function of patient age. The power management module 114 can control power output to light emitter panels to provide a therapeutically beneficial dose of illumination while still extending battery life.

Autism Spectrum Disorder (ASD) is a neurodevelopmental disorder characterized by diminished social functioning, inattentiveness, and linguistic impairment. While autism is likely to be a multi-causal disorder, research indicates that individuals with ASD frequently have mitochondrial disease which results in abnormalities of energy generation from food proteins. However, mitochondria in the brain might be able to produce energy molecules from a different source, such as light.

Using the wearable device 50, certain methods of the present disclosure may perform photobiomodulation (stimulating brain with light) and linguistic training simultaneously to treat children with ASD. The wearable device 50 may include several near infrared and/or red lights to stimulate the language area of the brain. These methods associated with the wearable device 50 may include determining an area of the head to position the wearable device 50 (e.g., the temporal lobe, the prefrontal cortex, and/or the occipital lobe) to output the infrared and/or red lights. The light absorbed by the brain tissue may increase the production of ATP, which may provide the neurons more energy to communicate with each other and provide increased brain connectedness. The wearable device 50 may simultaneously receive linguistic inputs from an application of a user device that is transmitted to the user via the headphones of the wearable device 50. The linguistic inputs may help facilitate language learning. Therefore, by providing these combined mechanisms (photobiomodulation and linguistic input), for example, to children diagnosed with ASD, may significantly improve lifelong outcomes. Further, the wearable device 50 may output meditations that may help reduce anxiety of the patient user (such as an ASD child), which may allow the user to better learn language and integrate socially.

Autism spectrum disorders are associated with brain inflammation, in particular, inflammation characterized by activation of brain macrophages (microglial activation) (see, e.g., Rodriguez et al., Neuron Glia Biol 2011, 7 (204): 205-213; Suzuki et al., JAMA Psychiatry 2013, 70 (1): 49-58; and Takano, Dev Neurosci 2015, 37:195-202, the entire contents of each of which are hereby incorporated herein by reference). Brain inflammation can be identified by the presence of delta waves (high voltage slow waves) during wakefulness that can be detected using electroencephalography (EEG). In healthy individuals, delta waves in EEG are detected during the period of restorative sleep, but not during wakefulness. Wakeful delta waves in EEG are associated with pathological conditions that are, in turn, associated with brain injury and inflammation characterized by activation of brain macrophages (microglial activation). Such pathological conditions include, among others, classical mitochondrial diseases like Alpers syndrome, traumatic brain injury and autism spectrum disorders (ASD). The presence of wakeful delta waves in an individual with a pathological condition described above indicates that healing activities normally confined to sleep were not sufficient for inhibiting brain inflammation.

Evidence also indicates that wakeful delta wave power is a reliable marker of brain inflammation and microglial activation. For example, symptomatic improvement in traumatic brain injury and genetic forms of mitochondrial brain disease is accompanied by a decrease in wakeful delta wave power. The presence of wakeful delta waves in an individual with ASD is indicative of brain inflammation.

Activation of microglia during brain inflammation results in synthesis and release of nitric oxide (NO) in the inflamed brain tissues. NO inhibits oxidative phosphorylation in the mitochondria by binding to the iron and copper atoms present in the mitochondrial electron transport chain complex IV cytochrome oxidase and inhibiting its activity (see, e.g., Mason et al., PNAS 2006 103 (3): 708-713, the entire contents of which are hereby incorporated herein by reference). Decreased levels of oxidative phosphorylation result in the increased levels of dissolved oxygen in the cell which, in turn, results in the increased levels of reactive oxygen species (ROS) and mitochondrial damage and fragmentation.

Near infrared light penetrates biological tissues, including bone structures such as the cranium. It can act to displace NO bound to the complex IV cytochrome oxidase, thereby reversing the effect of elevated NO levels and reversing the inhibition of oxidative phosphorylation. Restoration of mitochondrial oxygen consumption has the effect of stimulating healing of the inflamed tissues. Thus, without being bound by a specific pathway when other physiologic pathways, medications or therapeutic agents may alter the circumstances impacting treatment of a particular patient, it is believed that illuminating brain tissue of a subject with near infrared light can reverse inhibition of oxidative phosphorylation mediated by NO, stimulate oxidative phosphorylation and facilitate healing of inflamed brain tissues, thereby reducing brain inflammation. It is also believed, without being bound by a specific pathway as noted above, that the reduced inflammation of brain tissue resulting from illumination as described in the present application with red, near infrared light and/or infrared portions of the electromagnetic spectrum can cause a decrease in the wakeful delta waves, for example. Indeed, as indicated by the results of the clinical trial described herein, photobiomodulation therapy resulted in a statistically significant decrease in the delta waves in the treatment group as compared to a control group, which, in turn, was associated with a statistically significant reduction in autism symptoms.

Methods for providing cross-modal brain stimulation may include determining the light frequency, location of the LED lights (e.g., areas of the brain needing increased ATP, areas of the brain most likely to respond to light treatments, and/or areas of the brain associated with language (e.g., auditory cortex, Broca area, Wernike area)), whether ATP production increased, and the overall effect of the treatments. Accordingly, based on the determined overall effect on the brain, the wearable device may be dynamically adjusted on a user-specific basis.

The wearable device 50 may be specifically tailored for children with ASD, such that it improves language skills, alleviates anxiety, and/or reduces tantrums. Further, the wearable device 50 may be used on a daily basis, in the convenience of the family's home, without a need for a specially trained therapist. Moreover, the wearable device 50 may be non-invasive, may not require a prescription, and/or may lack side effects.

Methods for using the devices of the present disclosure may further include determining the location(s) of the light emitting diodes that may be used to stimulate specific brain areas responsible for language, comprehension, energy production, and/or for self-regulation (e.g., reducing anxiety). The methods may also include determining total power, power density, pulsing, and/or frequency. The total power may be 400-600 mW (0.4-0.6 W) with 100-150 mW per each of four panels. The power for each panel may be selectively stepped down to the 50-100 mW range, or increased to the 150-200 mW range depending on the age or condition of the patient. Each of these ranges may be further incremented in 10 mW steps during a treatment session or between sessions. The spot size of the light generated by each LED or laser can optionally be controlled by adjusting the spacing between the light emission aperture of the LED or by using a movable lens for one or more LEDs on each circuit board that can be moved between adjustable positions by a MEMS actuator, for example.

Further, the wearable device 50 may be comprised of a comfortable material for prospective patients. For example, the wearable device may be comprised of plastic, fabric (e.g., cotton, polyether, rayon, etc.), and/or the like. Because ASD patients in particular are especially sensitive, the aforementioned materials may be integral in allowing ASD patients to wear it for a sufficient amount of time without being irritable. Of course, the wearable device 50 may need to be both safe and comfortable. The electric components (e.g., processors, microphones, headphones, etc.) may be sewn into the wearable device 50 and may be difficult to reach by children, for example. A cloth or fabric covering can contain the head worn frame and optoelectronic components to the extent possible without interfering with the optical coupling of the LED to the cranium. Further, the weight of the wearable device 100 may be light enough to allow it to be worn comfortably. Moreover, the wearable device 100 may require a power source (e.g., one or more replaceable batteries) that allows it to be portable.

Regarding the linguistic inputs, a patient user device (e.g., a smartphone or tablet) may include an application that 1) performs language acquisition: (e.g., develops and records a vast number of short vignettes specifically designed to make syntactic structure transparent and teaches how to parse sentences); 2) involves a system of specifically designed mediations to alleviate anxiety; and 3) involves a system of musical rewards to keep users (children) interested and engaged.

The application may disambiguate syntactic structure of a language. Present research suggests that word learning spurt occurs after the children learn basic syntax (and it occurs at the syntactic-lexicon interface). Furthermore, without syntax children may not move beyond speaking 10-15 words, which may be used for simple labeling, but not to express their needs, wants and feelings. This means that there may be no ability for proper communication without learning syntax first. In addition, syntax may be necessary to parse the acoustic wave or sound that children hear into sentences and words. Syntax may also be necessary for specific word-learning strategies (e.g., syntactic bootstrapping).

Syntactic bootstrapping is a mechanism which children use to infer meanings of verbs from the syntactic clues. For example, when a child hears “Michael eats soup” this child infers that “eats” is a transitive verb. A classic example used by a famous psycholinguist professor Leila Gleitman is the made-up verb “Derk”. By putting this verb in several syntactic contexts, the meaning of the verb becomes transparent “Derk! Derk up! Derk here! Derk at me! Derk what you did!” Dr. Gleitman argued that children infer the meanings of verbs from hearing them in different syntactic contexts. In addition, Dr. Pinker argued that children also use semantic bootstrapping (contextual clues) to infer meanings of the words. Therefore, there are several mechanisms (most likely innate) available to a typical child while learning language. Overall, there is scientific consensus that typical children learn language by specifically focusing on syntactic and semantic clues of speech.

However, studies suggest that children who are on the autism spectrum cannot always extract syntactic structure and semantic contexts from the imperfect linguistic input they receive. Usual linguistic input is too messy, incomplete and confusing for them. People frequently speak in fragments of sentences, switch between topics, use incorrect words or use words in incorrect forms. Human speech may be too messy to allow for simple learning based on this type of speech alone. Neurotypical children can still extract syntactic structure from this messy input by being predisposed to pay attention to specific syntactic cues (e.g., to look for nouns and verbs in the string of speech). When children grasp syntactic structure of a language, they learn to parse sentences, and therefore, acquire more words. Several studies corroborated this hypothesis that massive word learning happens at this syntax-lexicon interface, including studies with children on the spectrum.

Many children suffering from ASD seem to be unable to move beyond simple labeling, are unable to speak in full sentences, and therefore are unable to communicate effectively. There are many reasons for this difficulty, one of them is that those children do not usually pay enough attention to speech and communication, and therefore they do not pay enough attention to syntactic clues and are not able to parse individual sentences. However, without grasping syntactic structure of the language, word learning beyond simple labeling becomes impossible, specifically, acquisition of verbs may become impossible. Timely acquisition of verbs (not just nouns to label objects around them) may be critical for ASD children, as research shows that the best predictor of future integration with the neuro-typical community (and normal functioning) is speaking full sentences by 5 years of age. Therefore, specifically, the problem is that children with ASD are not focused on the language enough to extract syntactic features of words, to parse sentences and to attend to syntactic and semantic clues of speech. Therefore, their word learning is delayed.

Accordingly, the aforementioned application may calibrate the imperfect linguistic input for ASD children, thus, making syntactic structure as transparent as possible. For example, the child will hear a noun: “dog”, then she will hear “1 dog, 2 dogs, 3 dogs, 4 dogs, 5 dogs”. then she will hear “my dog is brown”, “my brown dog is cute”, “my brown dog is small”, “I have a small, cute, brown dog”, “my dog barks,” “dogs bark” “dogs chase cats”, “dogs eat meat”, “I have a dog,” and so on. By putting the same word in different syntactic contexts over and over again we will flood the child with the information about linguistic markers (syntactic roles in the sentences, countable, noun, animate/inanimate and so on).

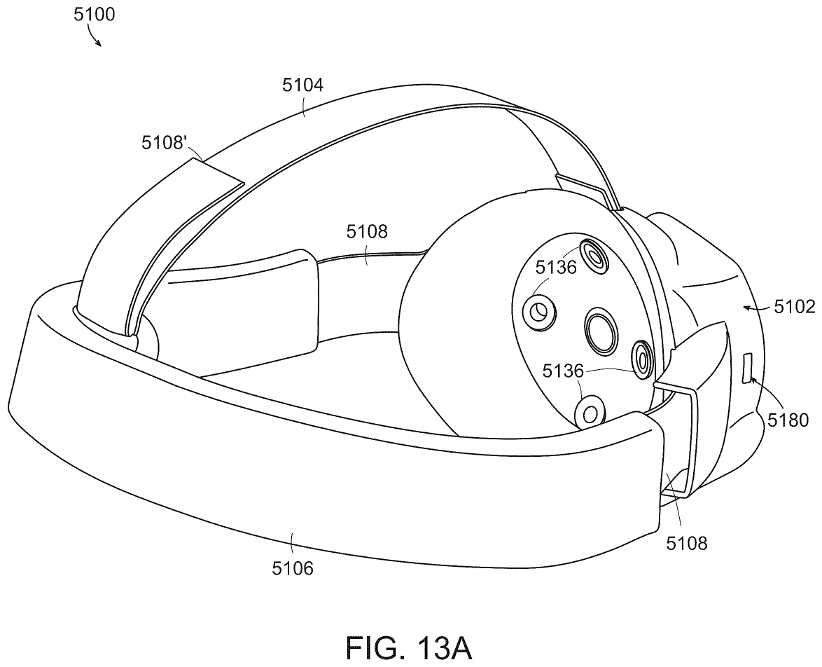

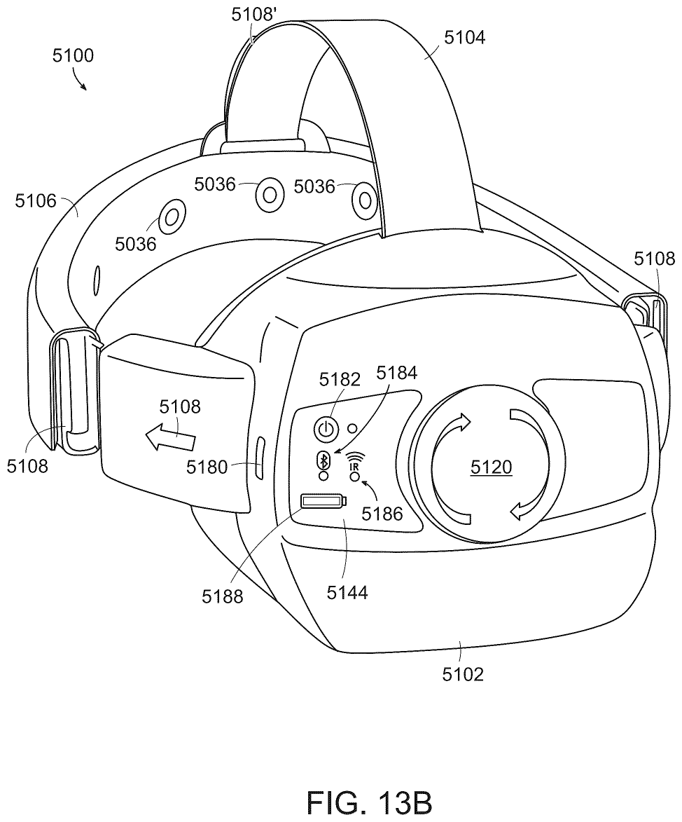

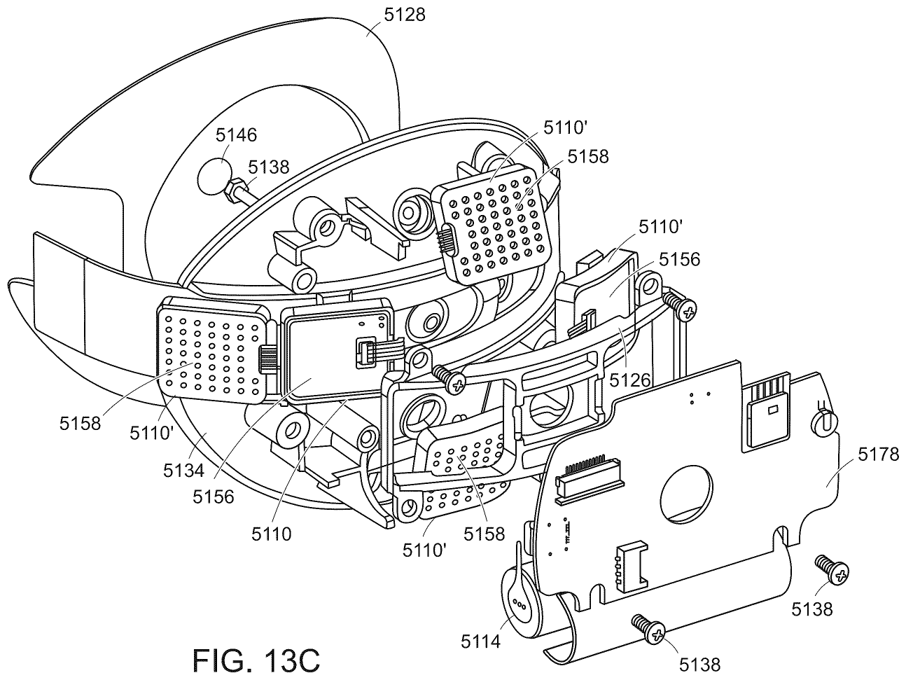

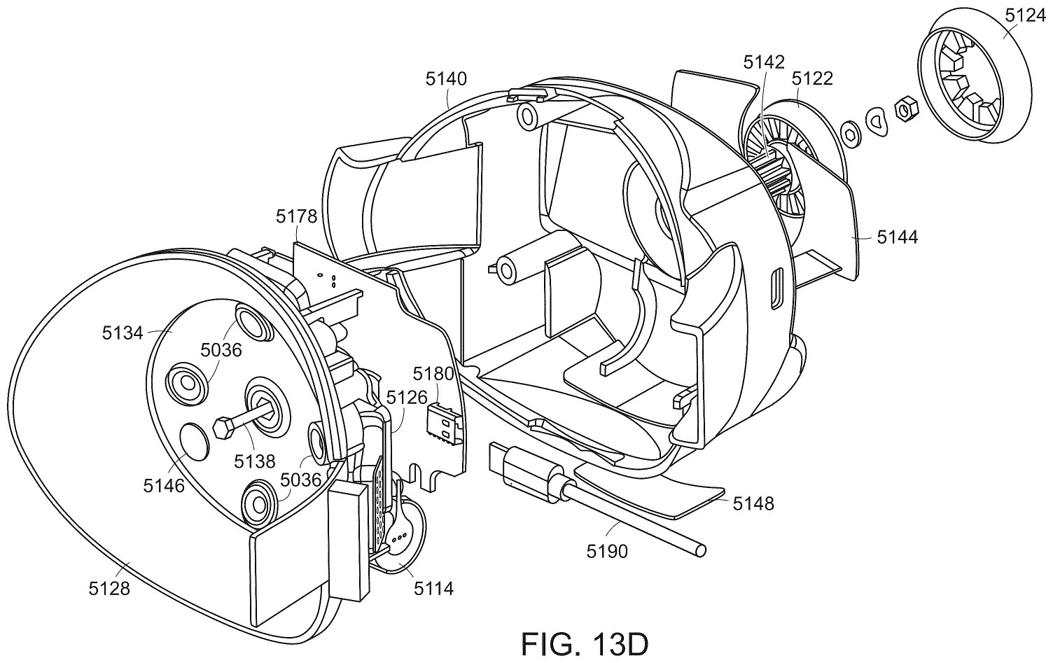

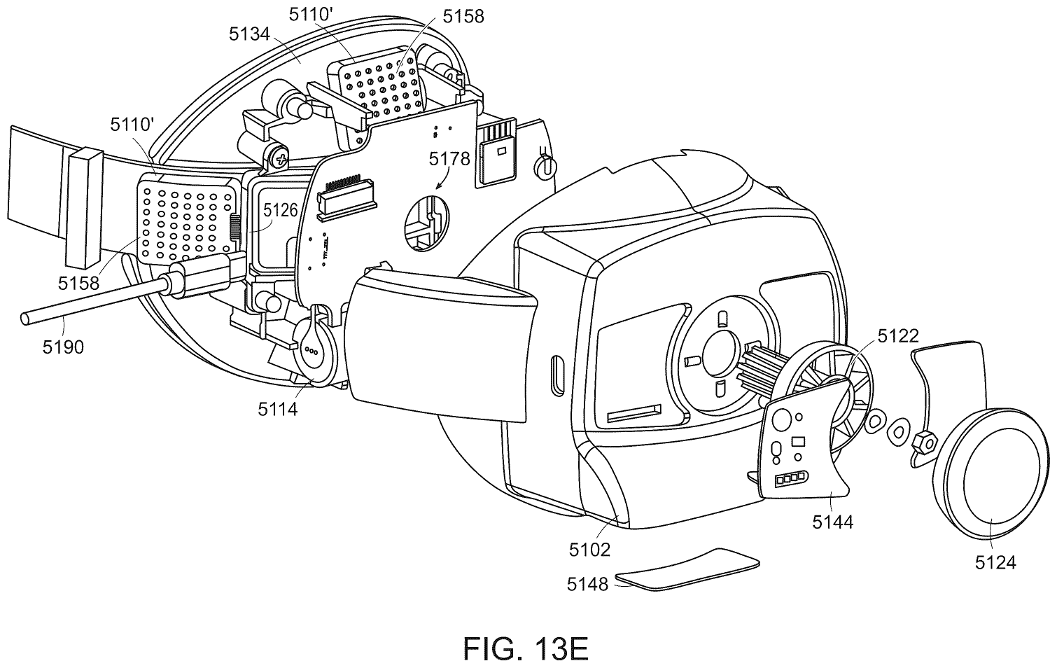

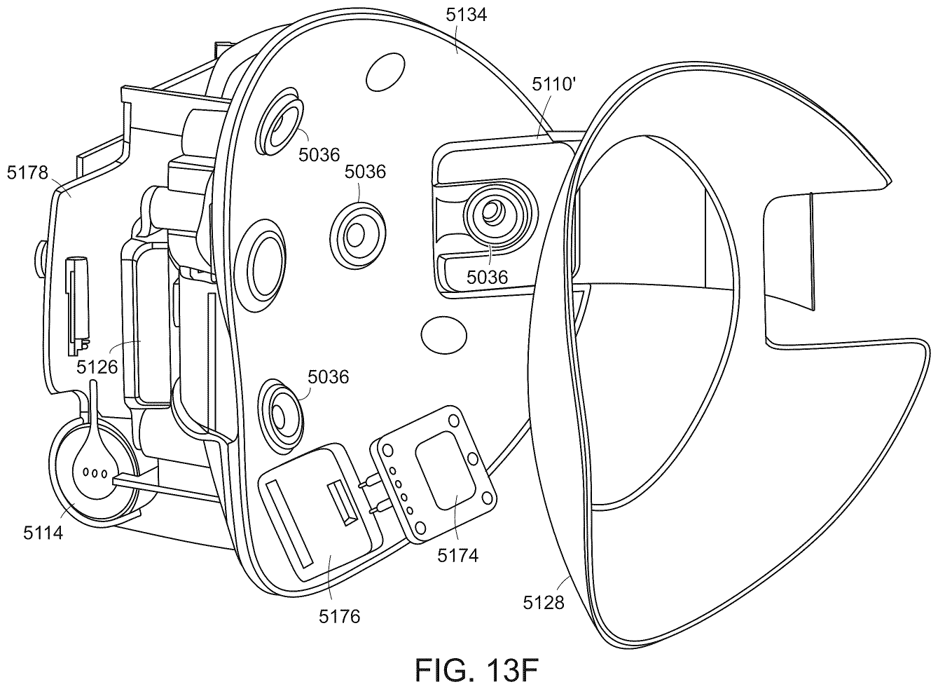

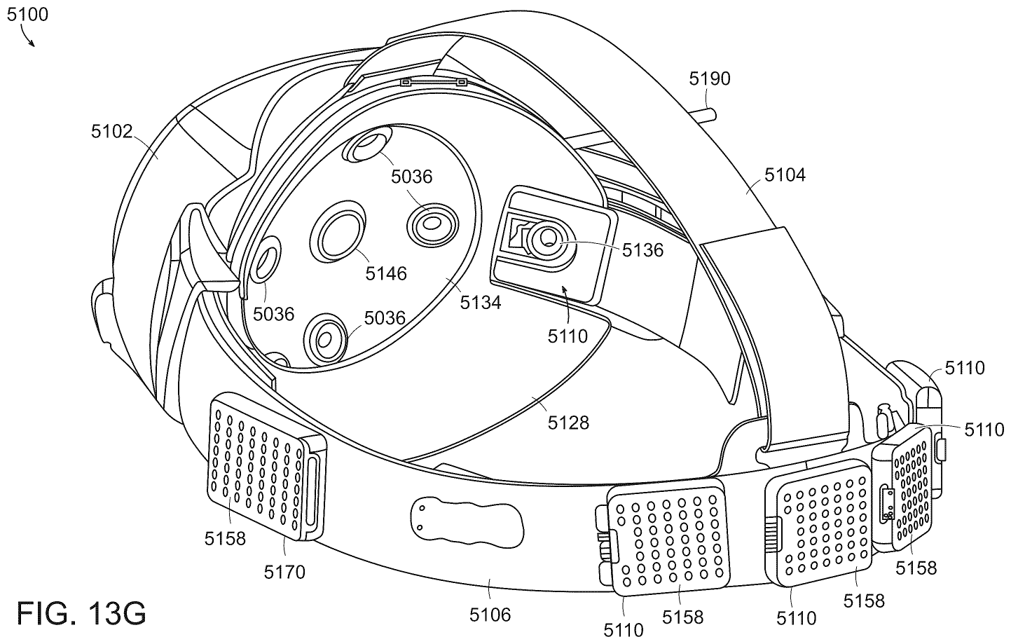

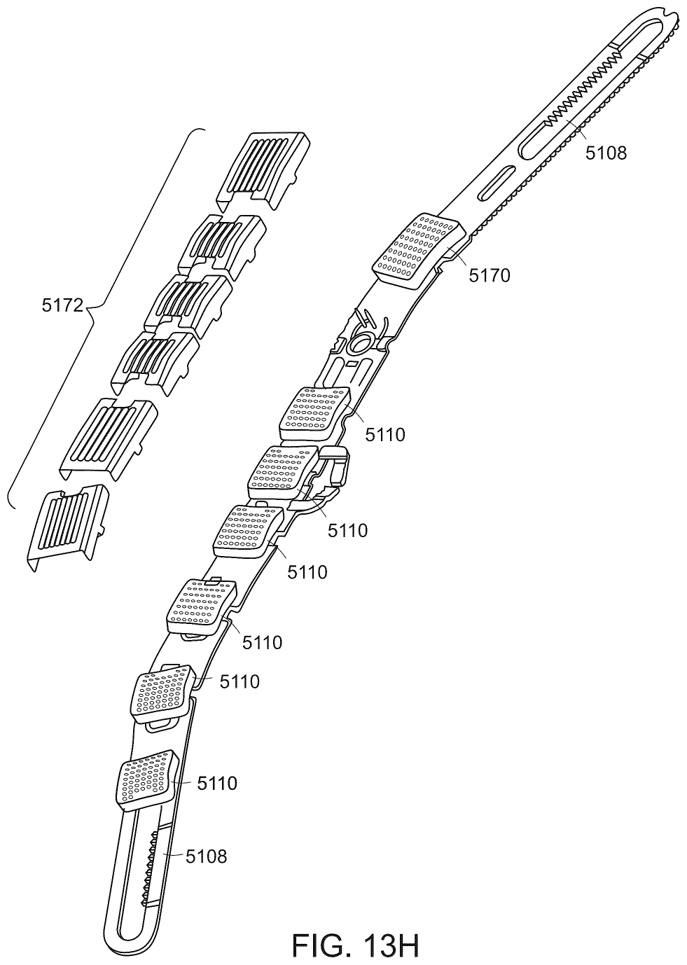

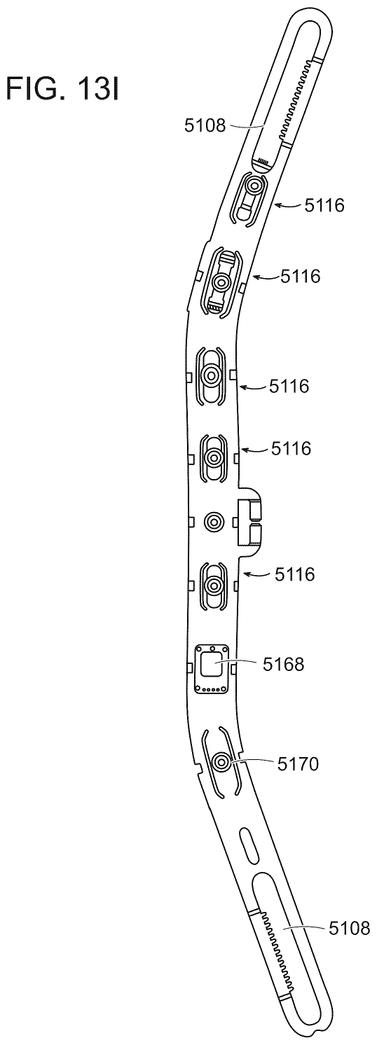

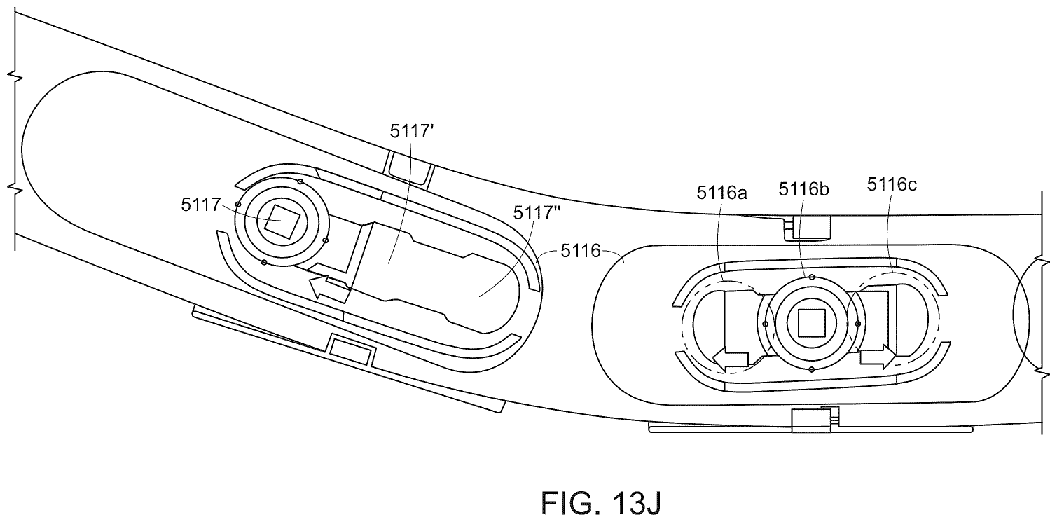

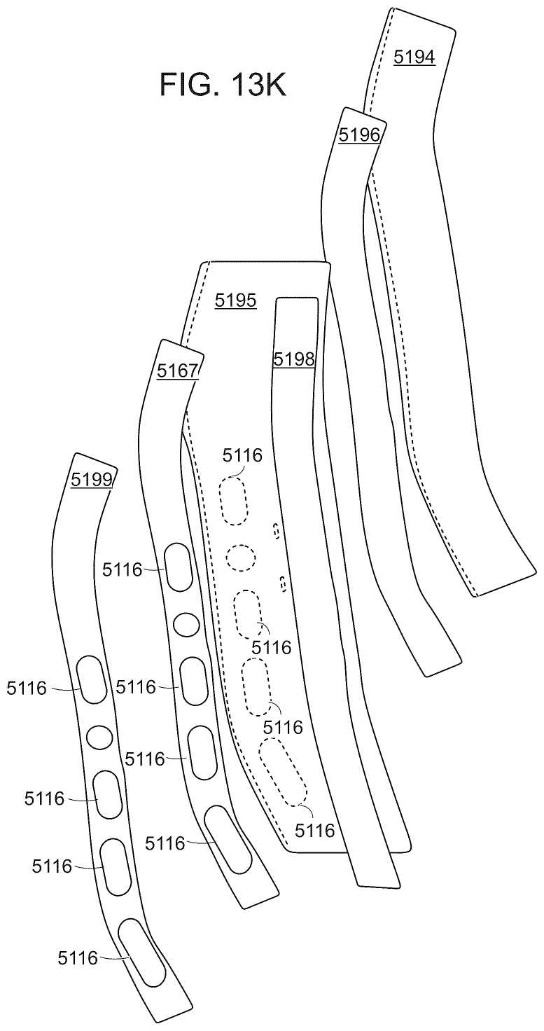

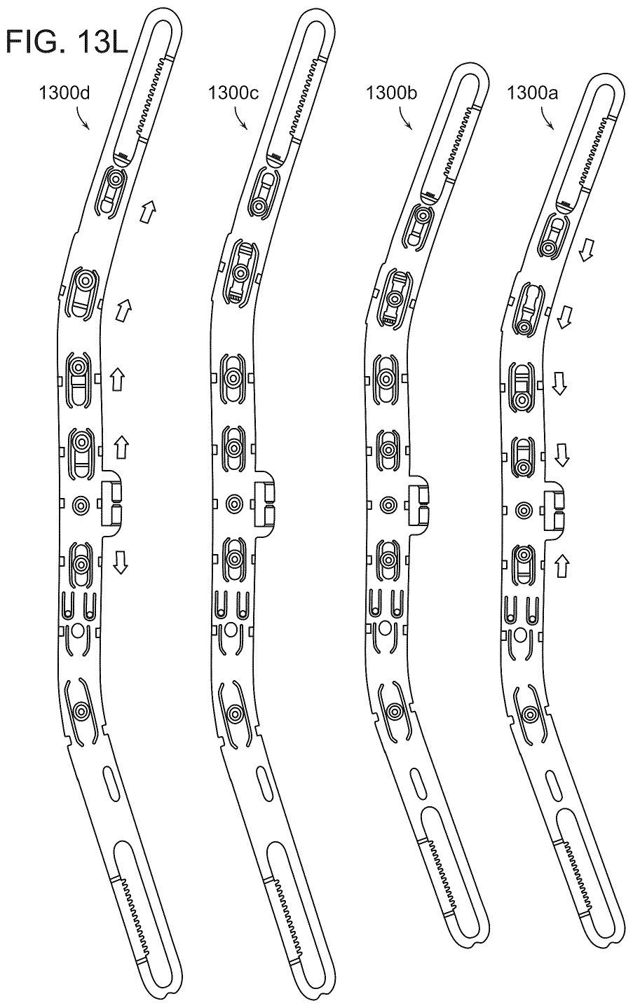

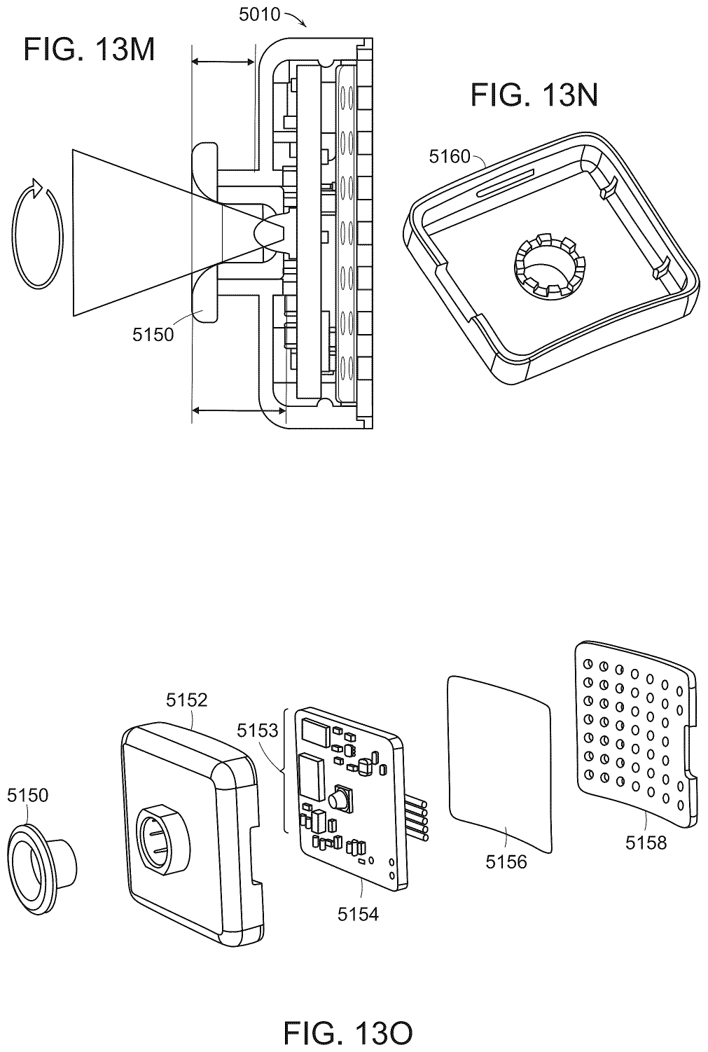

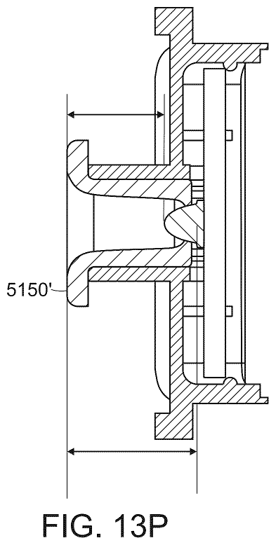

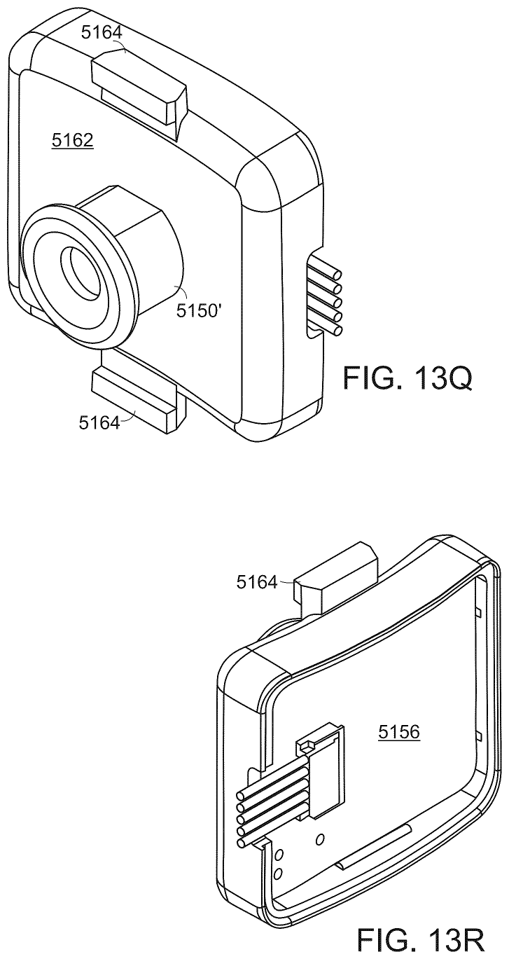

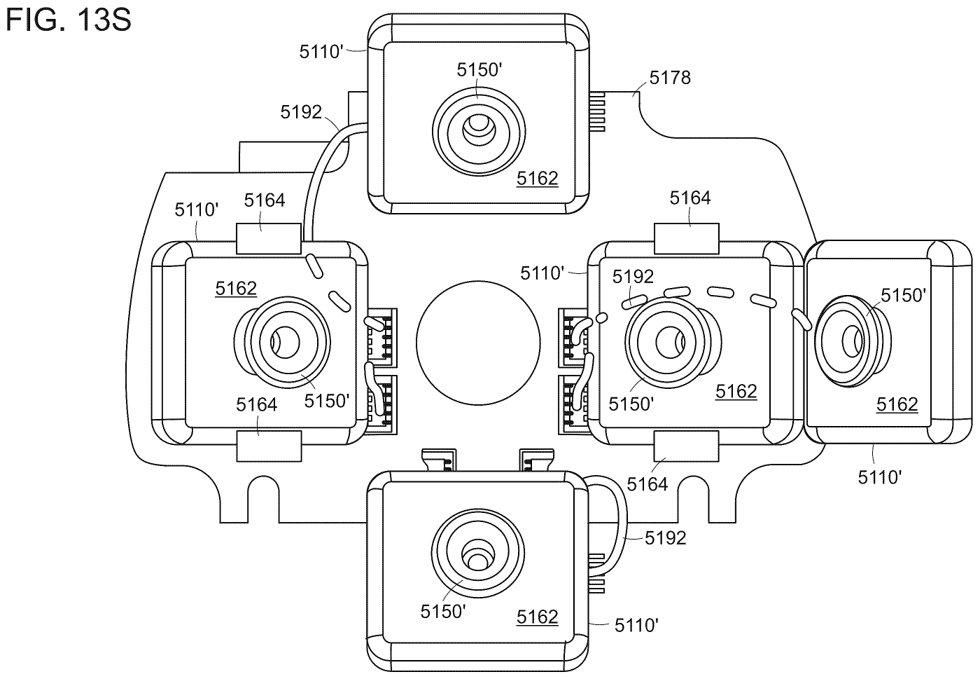

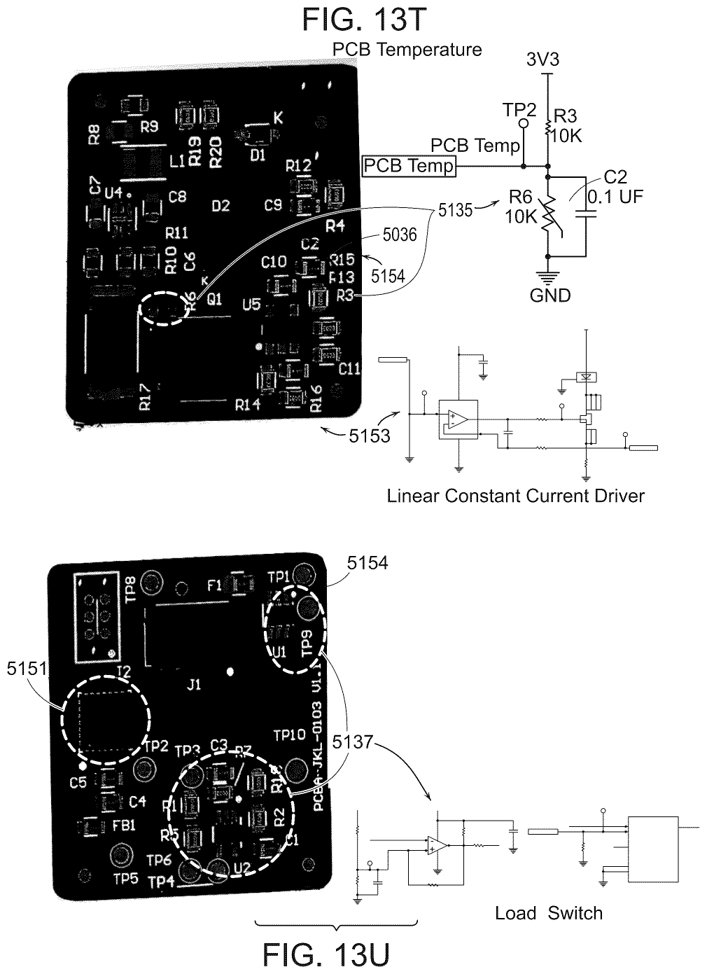

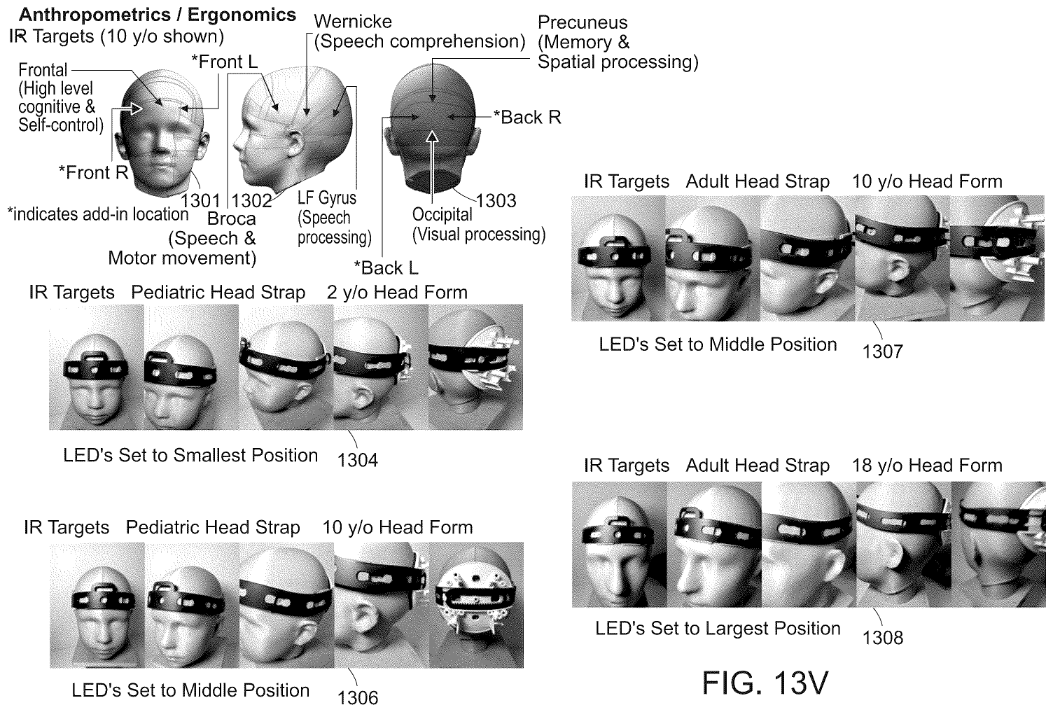

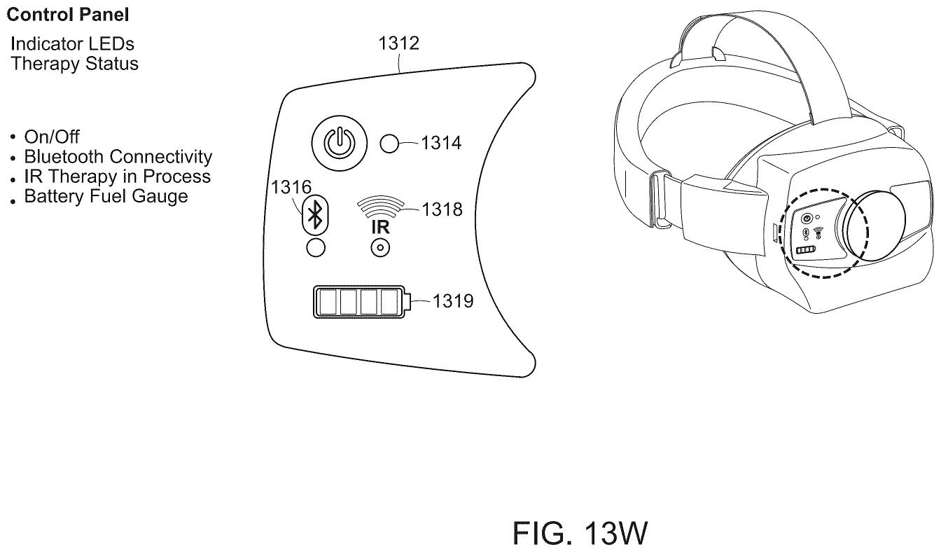

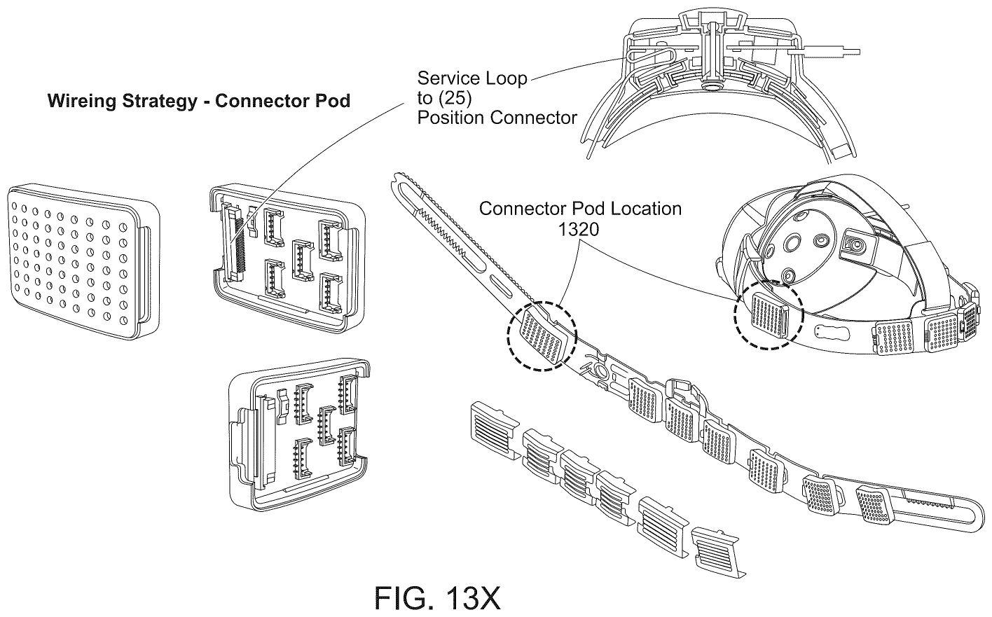

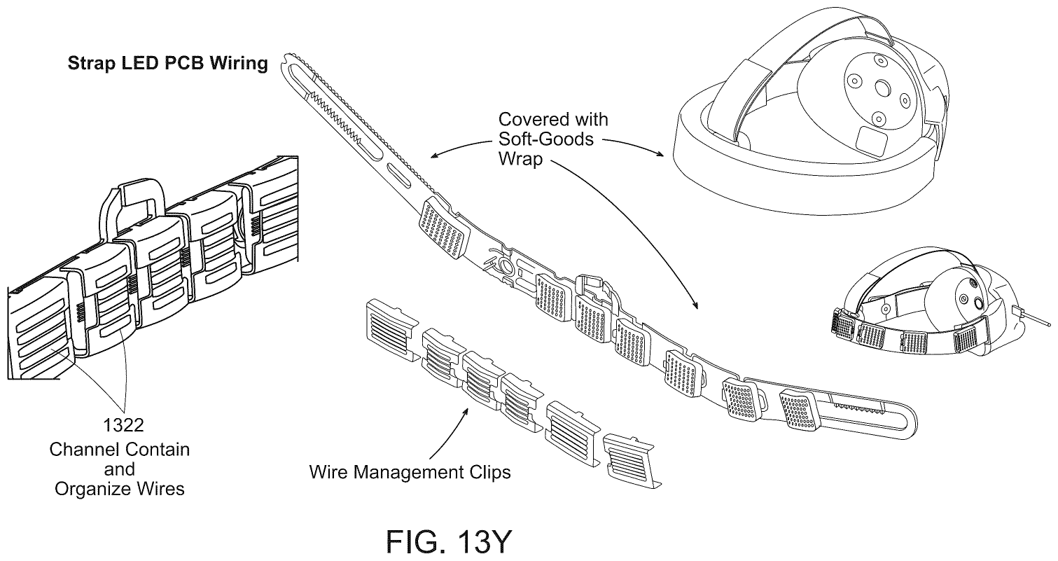

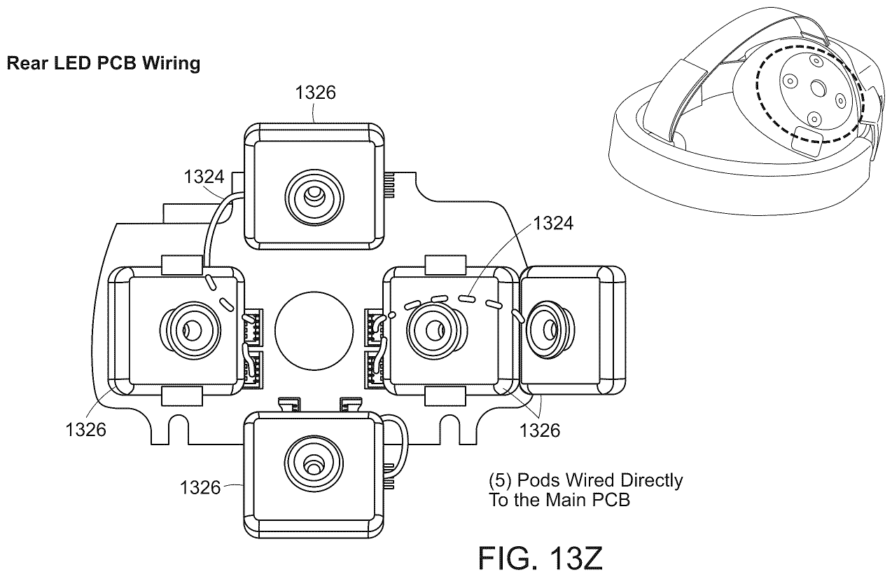

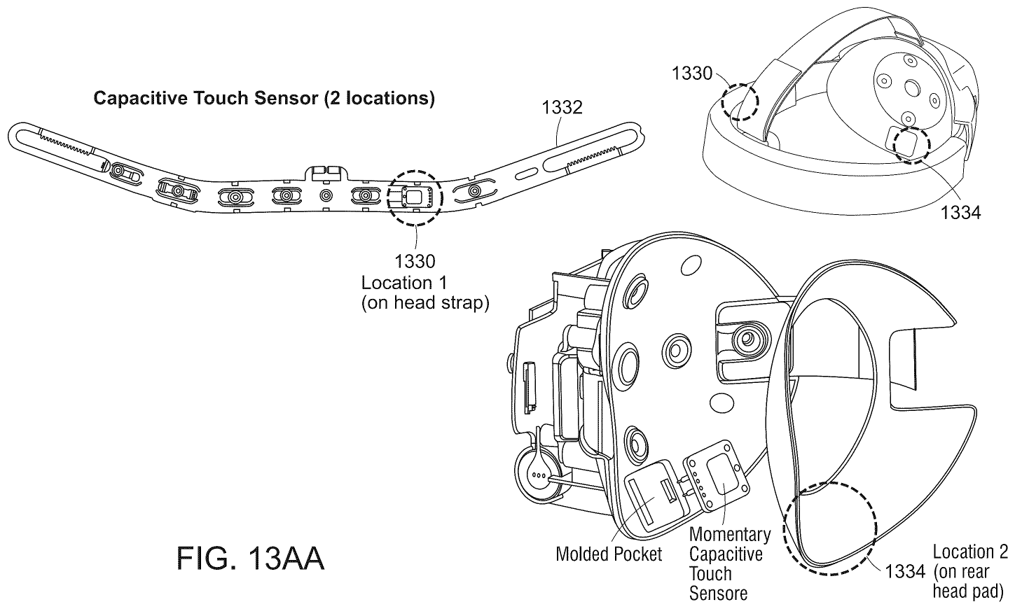

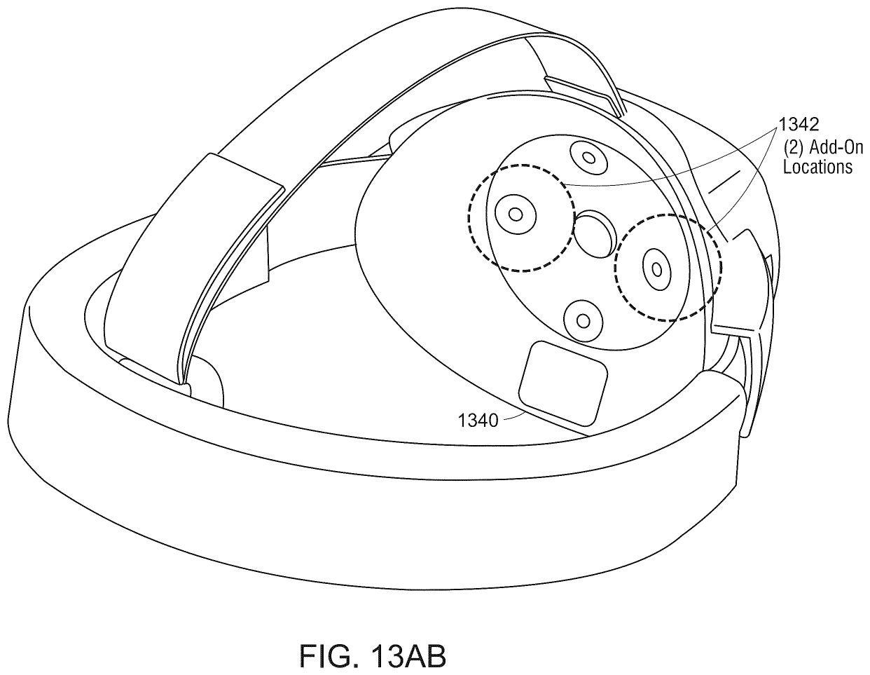

Therefore, the application may “wake up” (activate) language learning and make a child pay attention to the syntactic cues of the linguistic input. Further, the application may be refined by observing the behavior of the users and recording their improvements. A method for treatment 450 is described in connection with the process flow diagram of FIG. 13 wherein preset or manually entered parameters 452 can be entered by touch actuation on the tablet touchscreen so that the system controller can actuate the illumination sequence. These parameters are stored 454 in memory. The software for the system then executes stored instructions based on the selected parameters to provide transcranial illumination 456 for the therapeutic period. The system can utilize optional audio or video files 458 in conjunction with the therapy session. The system than communicates the recorded data 460 for the therapeutic session for storage in the electronic medical record of the patient. The data can be used for further analysis such as by application of a machine learning program to provide training data.

The following describes an example of a battery powered system as previously described herein where one or two 9 volt batteries are inserted into battery holders in the side and rear views showing the LED case design shown in the figures.

If the LED is uncased, a small tube can be used to ensure that it remains centered and held securely in place. This tube can fit through a hole in the foam band for proper location and is ⅜″ outside diameter. The PCB serves as a backing on the foam and allows clearance for the connecting cable. The same type of construction can be applied to the electronics mounted area, the battery, and the speakers. Sensors used to measure characteristics of the patient during use, such as EEG electrodes, photodetectors and/or temperature sensors can be mounted to the circuit boards carrying the LEDs or laser diodes as described generally herein. Detected electrical signals from the sensors can be routed to the controller board and stored in local memory and can also be transmitted via wireless transmission to the external tablet device so that a user or clinician can monitor the therapeutic session and control changes to the operating parameters of the system during use.

The electronics can comprise three or more separate PCB configurations with the LED PCB having (6) variations for the associated positions on the head. There can be two LED PCB boards on each side (front and rear) with at least one illuminating the temporal lobe on each side and at least one board centered for illuminating the frontal lobe. One or two boards can conform to one or both of the parietal lobe and the occipital lobe.

The system is fitted on the head of a patient and radiates energy via IR LEDs at 40 Hz into the patient's head, for example. The IR LEDs are split into six boards with each containing one IR LED. The LED utilized for preferred embodiments can be the SST-05-IR-B40-K850.

The LED boards can illuminate during the on-time of the 40 Hz signal. The duty cycle of the 40 Hz signal will be equal to the power setting. For example, a power setting of 25% will require a 25% duty cycle for the 40 Hz.

One or more 9V batteries can be the system's source of power. A buck converter reduces the 9V from the battery to 2.5V for the LEDs. One or more batteries of different voltages can be employed particularly where different batteries can be used for the light emitters and powering the circuitry.

In this section, note the calculation of the LED's absolute maximum optical flux output assuming that they are the only components powered by a single 9V battery.

Table 1 shows the current limits of important components. These current limits cannot be violated without the risk of permanent damage to the component.

| | Current |
|---|---|
| | Limit |
| Device Specification | (A) |
| Absolute Maximum Battery | 1.0 |
| Discharge | |
| Absolute Maximum Buck | 2.5 |
| Converter Current Output | |
| Absolute Maximum IR LED | 1.0 |
| Current | |

Conservation of energy dictates that the current sourced by the buck converter will not be the same as the current sourced by the battery. Equation 1 calculates the current drawn from the battery (IBATT).

$${I}_{BATT} = \frac{{V}_{LED \_ PWR} \times {I}_{LED \_ PWR}}{η \times {V}_{BATT}}$$

where V_{LED_PWR} is the LED supply voltage (2.5 V), I_{LED_PWR} is the buck converter output current, η is the efficiency (minimum of 0.85), and V_{BATT} is the battery voltage.

The efficiency of the buck converter changes over the output current range. The minimum efficiency is 0.85 at the maximum current of 2.5 A.

Note that the battery voltage is inversely proportional to the battery draw. For a fixed load, the battery will draw more current as the battery discharges. Therefore, a minimum battery voltage must be specified and observed by the system microcontroller to avoid exceeding the battery's maximum discharge current. Table 2 demonstrates how battery current draw increases as the battery discharges. Each battery draw value is calculated with Equation 1 with the following values: η=0.85, V_{LED_PWR}=2.5V, I_{LED_PWR}=2.5 A, and the battery voltage for V_{BATT}.

| | Battery |
|---|---|
| | Draw |
| Scenario | (mA) |
| Fully Charged Battery at 9 V | 816 |
| Intermediate Charge at 7.5 V | 980 |
| Absolute Minimum | 1000 |
| Battery Voltage, 7.35 V | |

Use Equation 2 to calculate the absolute minimum battery voltage, V_{B_AM}. Use the same values as before, but let I_{B_MAX}=1. Battery current draw reaches 1.0 A when the battery voltage discharges to 7.35V, therefore the LEDs must be turned off to avoid exceeding the L522 battery maximum discharge specification of 1.0 A. The buck converter supplying 2.5 A at 2.5V with a battery voltage below 7.35V risks permanent damage to the battery.

$${V}_{B \_ AM} = \frac{{V}_{LED \_ PWR} \times {I}_{LED \_ PWR}}{η \times {I}_{B \_ MAX}}$$

### Equation 2. Absolute Minimum Battery Current Draw

The absolute minimum battery voltage also affects battery life. FIG. 25 illustrates a discharge curve for the (Energizer L522) battery and it demonstrates that a lower absolute minimum battery voltage prolongs battery life. With a 7.35V absolute minimum battery voltage, the LEDs can be safely powered for approximately 24 minutes (if the battery was drawing 500 mA instead of 1.0 A). Thus, a lower absolute minimum battery voltage is beneficial.

The 2.5 A sourced by the buck converter must be shared amongst six boards (LED). Thus 2.5 A/6=416 mA from the buck converter per LED.

The duty cycle of the 40 Hz will attenuate the optical flux. Equation 3 shows how to calculate the average flux for a single pulsed LED. E_{e_pulse} is the optical flux during the pulse and D_{40 HZ} is the 40 Hz duty cycle. The range of D_{40 HZ} is a number between 0 and 1 inclusive.

$${E}_{e \_ average} = {D}_{40 HZ} \times {E}_{e \_ pulse}$$

### Equation 3. Average Irradiance for a Single LED.

As an example, Table 3 lists the optical flux for each power setting.

TABLE 3
Power Settings at Maximum
System Power.

| | Optical |
|---|---|
| | Flux |
| Power | Output |
| Setting | (mW) |
| 2% | 5.84 |
| 4% | 11.68 |
| 6% | 17.52 |
| 8% | 23.36 |
| 16% | 46.72 |
| 25% | 73 |
| 50% | 146 |
| 100% | 292 |

The output optical flux decreases with temperature and must be de-rated accordingly. Sources of heat to take into account are the LEDs' self-heating and the heat from the patient's head. For the purposes of this analysis, assume the patient's head is at body temperature, 37° C.

TABLE 4
Temperature Related Coefficients

| Parameter | Value |
|---|---|
| Temperature Coefficient of | −0.3%/° C. |
| Radiometric Power | |
| Electrical Thermal Resistance | 9.2° C./W |

Table 4 above lists two thermal coefficients. The thermal resistance of the LED can be understood as for every watt consumed by the LED, its temperature will rise by 9.2° C. The third graph below shows normalized V-I characteristics of the LED relative to 350 mA at 2V (at 350 mA, forward voltage ranges between 1.2V and 2.0V, but here we continue to use worst-case value of 2.0V).

At 416 mA (the maximum current available per LED), the forward voltage is approximately 2V+0.04V=2.04V. Using Equation 4, the temperature rise due to self-heating is 7.8° C. at a 100% 40 Hz duty cycle.

$${T}_{{Δ}_{LED}} = {V}_{f} \times {I}_{f} \times {D}_{40 HZ} \times 9.2$$

### Equation 4. Temperature Rise Due to Self-Heating

The LED can rise to a temperature of T_{LED}=37° C.+7.8° C.=44.8° C. Using the temperature coefficient of radiant power from Table 4 and Equation 5, the change in radiant power due to temperature is −5.94%. Therefore, de-rating the worst-case optical flux of 292 mW derived above by 5.94% yields approximately 275 mW.

$${PO}_{Δ \%} = ( {T}_{LED} - 25 ° C . ) \times ( - 0.3 \% / ° C . )$$

### Equation 5. Change in Output Flux Due to Temperature.

Note that the system also provides a temperature coefficient for forward voltage. Forward voltage decreases with temperature rise. For a worst-case analysis, the decrease in forward voltage due to temperature can be ignored.

An optical flux of 275 mW is the minimum absolute maximum that can be achieved if the buck converter and the battery are pushed to their limits assuming that the battery is only supplying power to the LEDs.

Since the battery may also be powering the digital logic which includes the microcontroller, the Bluetooth module or other wireless connection, etc. the LEDs cannot draw the 1.0 A maximum from the battery.

The steps below are an effort to summarize the approach described above.
1. Start by selecting the target current for a single LED, I_{f}.
2. The current sourced by the buck converter will be I_{LED_PWR}=6×I_{f}. If I_{LED_PWR} exceeds 2.5 A, you must decrease I_{f}.
3. Use Equation 2 to calculate the minimum safe battery voltage to ensure desired battery life and safe operating conditions. For efficiency, either use the worst-case value of 0.85 or select the closest efficiency for your value of I_{LED_PWR} from Table 5.

TABLE 5
Buck Converter Efficiency for
Different Output Currents

| Buck | |
|---|---|
| Converter | |
| Output, | |
| I_{LED}_PWR | Efficiency, |
| (A) | η |
| 2.5 | 0.85 |
| 2.0 | 0.871 |
| 1.5 | 0.89 |
| 1.0 | 0.91 |
| 0.5 | 0.926 |
| 0.25 | 0.91 |
| 0.125 | 0.88 |

4. Use graph to approximate the optical flux output at I_{f}.
   a. Note: Graph is normalized to optical flux of 265 mW at 350 mA.
5. Use graph to approximate the forward voltage at I_{f}.
   a. Note: Graph is normalized to 2.0V forward voltage at 350 mA.
6. Calculate the self-heating temperature rise, T_{Δ_LED}, using Equation 4. Use D_{40 Hz}=1 for 100% 40 Hz duty cycle as the worst-case temperature rise.
7. De-rate the optical flux for a TA LED rise over ambient temperature. Use 37° C. for ambient temperature. This de-rated optical flux is the maximum flux output for a single LED.

Table 6 gives examples of target LED current and the resulting system specification. Allow a 100 mA margin on the battery draw for supply logic. Values calculated in Table 6 assume worst-case efficiency of 0.85.

TABLE 6
LED Current Target Examples.

| | | | | | Flux |
|---|---|---|---|---|---|
| | Absolute | | | Flux | Output |
| Target | Minimum | Battery | LED | Output | per LED |
| LED | Battery | Draw | Temperature | per | (temperature |
| Current | Voltage | at 6.5 V | Rise | LED | adjusted) |
| (mA) | (V) | (mA) | (° C.) | (mW) | (mW) |
| 100 | 1.765 | 271 | 1.7 | 57 | 55 |
| 200 | 3.529 | 543 | 3.5 | 133 | 127 |
| 300 | 5.294 | 814 | 5.4 | 207 | 196 |
| 339 | 5.982 | 900 | 6.2 | 236 | 223 |

The maximum target LED current is 339 mA resulting in a temperature adjusted flux output of 223 mW. Table 7 demonstrates how the 40 Hz duty cycle attenuates the LED output flux.

TABLE 7
Power Settings for Realistically
Maximizing Flux Output

| | Optical |
|---|---|
| | Flux |
| Power | Output |
| Setting | (mW) |
| 2% | 4.46 |
| 4% | 8.92 |
| 6% | 13.4 |
| 8% | 17.8 |
| 16% | 35.7 |
| 25% | 55.8 |
| 50% | 111.5 |
| 100% | 223 |

EEG can be used to augment the use of TPBM to reduce symptoms of autism, for example, and this procedure is described in further detail below.

The head wearable device reduces symptoms of autism by applying tPBM to stabilize functional brain connectivity, while using EEG data as a measure of the efficacy of tPBM and as a guide for continuous applications. The head wearable device can include EEG electrodes situated on one or more of the light emitter printed circuit boards as described herein. Between one and six EEG electrodes can be mounted on one or more of the light emitter panels so that they are interleaved between the light emitters or surround them so as to detect brain wave signals occurring during illumination.

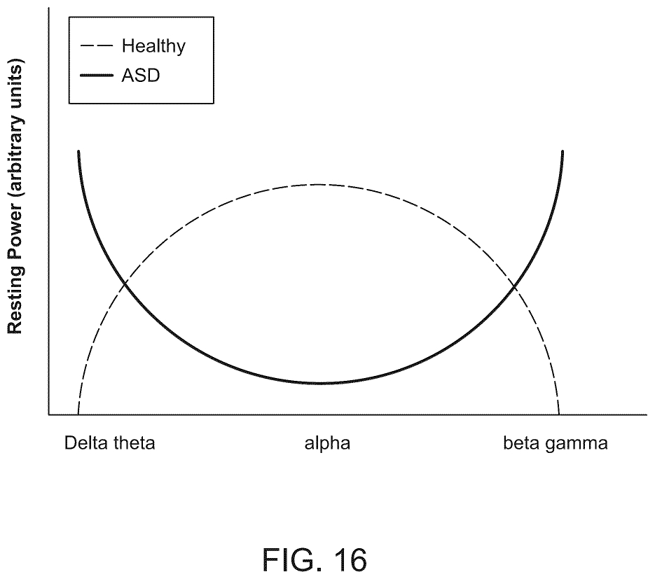

Autism (ASD) is a life-long disorder characterized by repetitive behaviors and deficiencies in verbal and non-verbal communication. Resent research identified early bio-markers of autism, including abnormalities in EEG of ASD infants, toddlers and children as compared to typical children. For example, children diagnosed with ASD present with significantly more epileptiforms (even, when they do not develop seizures), some researchers report as many as 30% of ASD children present with epileptiforms (e.g., Spence and Schneider, Pediatric Research 65, 599-606 (2009). A recent longitudinal study (from 3 to 36 months) detected abnormal developmental trajectory in delta and gamma frequencies, which allow distinguishing children with ASD diagnosis from others (Gabard-Durnam et al 2019). Short-range hyper-connectivity is also reported in ASD children. For example, Orekhova et al (2014). showed that alpha range hyper-connectivity in the frontal area at 14 months (and that it correlates with repetitive behaviors at 3 years old). Wang et al (2013), has indicated that individuals with ASD present with abnormal distribution of various brain waves. Specifically, the researchers argued that individuals with ASD show an excess power displayed in low-frequency (delta, theta) and high-frequency (beta, gamma) bands as shown in FIG. 16, and reduced relative and absolute power in middle-range (alpha) frequencies across many brain regions including the frontal, occipital, parietal, and temporal cortex. This pattern indicates a U-shaped profile of electrophysiological power alterations in ASD in which the extremities of the power spectrum are abnormally increased, while power in the middle frequencies is reduced.

Duffy & Als (2019) argued, based on EEG data, that ASD is not a spectrum but rather a “cluster” disorder (as they identified two separate clusters of ASD population) and Bosl et al Scientific Reports 8, 6828 (2018) used non-linear analyses of infant EEG data to predict autism for babies as young as 3 months. Further details concerning the application of computational methods of Bosl can be found in US Patent publication 2013/0178731 filed on Mar. 25, 2013 with application Ser. No. 13/816,645, from PCT/US2011/047561 filed on Aug. 12, 2011, the entire contents of which is incorporated herein by reference. This application describes the application of machine learning and computational techniques including the use of training data stored over time for numerous patients and conditions that can be used to train the a machine learning system for use with the methods and devices described herein. A neural network can be used for example to tune the parameters employed for transcranial illumination of a child at a certain age range undergoing treatment for autism. An array of 32 or 64 EEG channels can be used with electrodes distributed around the cranium of the child. Overall, the consensus is that ASD is a functional disconnectivity disorder, which has electrophysiological markers, which can be detected through an EEG system. Dickinson et al (2017) showed that at a group level, peak alpha frequency was decreased in ASD compared to TD children.

Transcranial photobiomodulation as described herein is used to treat many neurological conditions (TBI, Alzheimer, Depression, Anxiety), and is uniquely beneficial to autism, as it increases functional connectivity AND affects brain oscillations (Zombordi, et al, 2019; Wang et al 2018). Specifically, Zomorrodi et al Scientific Reports 9(1) 6309 (2019) showed that applying tPBM (LED-based device) to Default Mode Network increases a power of alpha, beta and gamma, while reduces the power of delta and theta (at resting state). Wang et al (2018) also showed significant increases in alpha and beta bands. Finally, Pruitt et all (2019) showed that tPBM increases cerebral metabolism of human brain (increasing ATP production).

Thus, preferred embodiments use a system that correlates continuously collected EEG data with observable symptoms (as reported by the parents) and use EEG to guide application of LED based tPBM. The symptoms provided by parents can provide ranked data can be used to formulate the parameters for a therapy session.

LED based tPBM can be applied to Default Mode Network (avoiding central midline areas) as well as Occipital lobe, and Broca area (left parietal lobe) as well as Wernike area (left temporal lobe).

Stimulating DMN (and simultaneous stimulation of frontal lobe with occipital lobe) increases long-range coherence. Stimulating language producing areas (e.g., Broca and Wernike areas with DMN) has been shown to facilitate language production in aphasic stroke patients (Naeser, 2014).

The device performs EEG measurements in combination with photobiomodulation therapy:
1. Analyze initial EEG data for epileptiforms, long-range coherence and hemispheric dominance.
2. Correlate EEG data with observed symptoms.
3. Based on the observed symptoms and the EEG data, the head wearable device can apply tPBM. For example, for children with severe repetitive behaviors and strong delta and theta power in the prefrontal cortex, the device stimulates prefrontal cortex to increase power within alpha and beta frequency band (and decrease power of delta and theta bands). For children who struggle with language, the device can stimulate DMN and Broca and Wernike areas. For children with various and severe symptoms, the device can stimulate all identified targeted areas (DMN, Broca, Wernike, occipital lobes).
4. The device can adjust power gradually and increasing it until the minimal change in brain oscillation is detected. This thresholding avoids applying too much power to a developing brain. The device operates at the lowest power that achieves the desired oscillation.
5. As the symptoms improve and the measured EEG signal stabilizes, the power level of the device can be gradually reduced. This system can be automated to control each therapy session.
6. Machine learning algorithms analyze EEG data and behavioral data, and the power alterations provided by the algorithm in the form of guidance to parents (and therapists), as well as indicate further improvements in the therapy being given to the patient.
7. As the symptoms sufficiently improve (expected improvement is within 8 weeks based on Leisman et al 2018), the device controls a break from tPBM and collect only EEG and behavioral symptoms to monitor for possible regression.
8. If any regress is detected, the device can instruct that tPBM is gradually resumed.

The device can apply tPBM to DMN, occipital lobe as well as to Broca and Wernike areas. The device collects EEG signals from prefrontal cortex, occipital cortex and temporal cortex (left and right to monitor hemispheric dominance observed in ASD children). The platform connected to the device can conduct initial assessment of behavioral symptoms (to be correlated with EEG data) as well as ongoing collection of symptoms (allowing for continuous correlations with EEG). Therefore the platform will continuously measure the efficacy of tPBM and personalization can be developed. Initially, a baseline is established by two separate measures. First, functional brain connectivity and brain oscillations baselines can be established in targeted brain areas (e.g., F1 & F2, T3 & T4, 01 & 02) prior to using treatment. Second, baseline demographic information (age, gender, race, etc), most concerning symptoms, and medical history (e.g., known genetic mutations, mitochondrial dysfunctions, gastroenterological symptoms, asthma, epilepsy, medications taken on regular basis) of each child is collected from parents. After the initial low-dosage treatment is administered several times (>3), a child's brain oscillations can be measured in order to establish the trend for reduction of delta brain waves, which is needed in order to detect the treatment's effect on the brain's electrophysiological activity and penetration of light through the skull. Separately, data can be collected from parents about the child's behavioral symptoms, including language, responsiveness, aggression, self-injurious behavior, irritability, and sleep disturbances. An AI algorithm, described in further detail below, processes collected data to determine the combination of effectiveness (as marked by behavioral symptoms and EEG data) as well as tolerability (as marked by behavior, reported by parents) to compute an optimal dosage (which include total power, time administered and frequency of pulsing). For example, the device used in some embodiments uses 40 HZ pulsing, which usually increases focus. However, for hyper-active children 10 HZ pulsing or continuous wave administration can be used and can be effective. To further improve personalization features, the system can be programmed to adjust for skin color based on the timing and strength of dosage. Darker skin pigments absorb light more than lighter skin, therefore fewer photons are likely to reach the brain. In the clinical study, children with darker skin showed improvement later than children with lighter skin, thereby indicating a need to adjust dosage based on skin absorption. Therefore, they might need longer usage of the device at a given dosage to detect improvements. The control software for the device can be programmed for such personal characteristics as race and ethnicity.

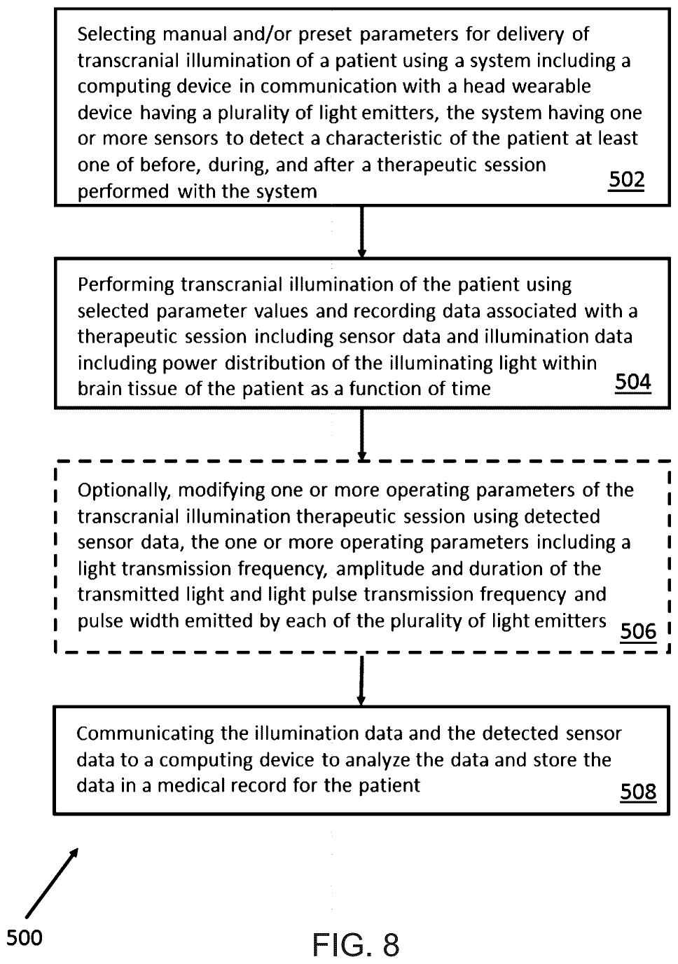

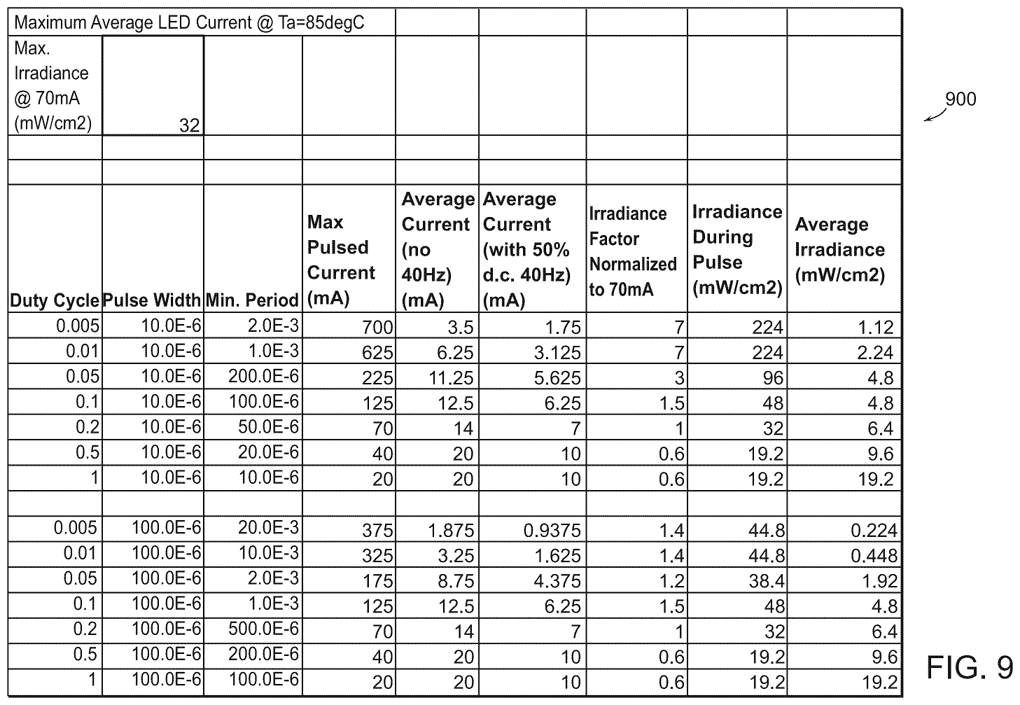

The process flow diagram in FIG. 8 illustrates the method 500 of performing transcranial illumination in combination with the use of one or more sensors to measure characteristics of the brain to monitor the treatment and detect changes in tissue that indicate a response during one or more sessions. Preferred embodiments can utilize an EEG sensor array with the head wearable device to measure brain electric field conditions where manual or preset parameters are selected 502 for a therapeutic session. The system performs transcranial illumination 504 and data is recorded such as EEG sensor data. Depending on the measured data and condition of the patient, the system can automatically adjust operating parameters or they can be manually adjusted 506 by the clinician. The data can be communicated 508 to the computing device such as the control tablet device and stored in the electronic medical record of the patient. This can be transmitted by communication networks to a hospital or clinic server for storage and further analysis as described herein. Shown in FIG. 9 is a table 900a with exemplary values for illumination conditions that can be employed by the system. These parameters typically fall within a range of values that the system can use that extend between a minimum threshold and a maximum threshold. These thresholds can be age dependent as the thickness and density of the cranium of a child increase with age as described in Smith et al, “Automated Measurement of Cranial Bone Thickness and Density from Clinical Computed Tomography,” IEEE conference proceedings Eng Med Biol Soc. 2012:4462-4465 (EMBC 2012), the entire contents of which is incorporated herein by reference. Thus, an age dependent quantitative rating can be associated with each patient that is used to define the illumination parameters used for that patient. Note that different lobes of a child may increase in thickness and/or density at different rates over time. Thus, the power density to be delivered to a child at age 4 will be less than that used for a 5 or 6 year old, for example.

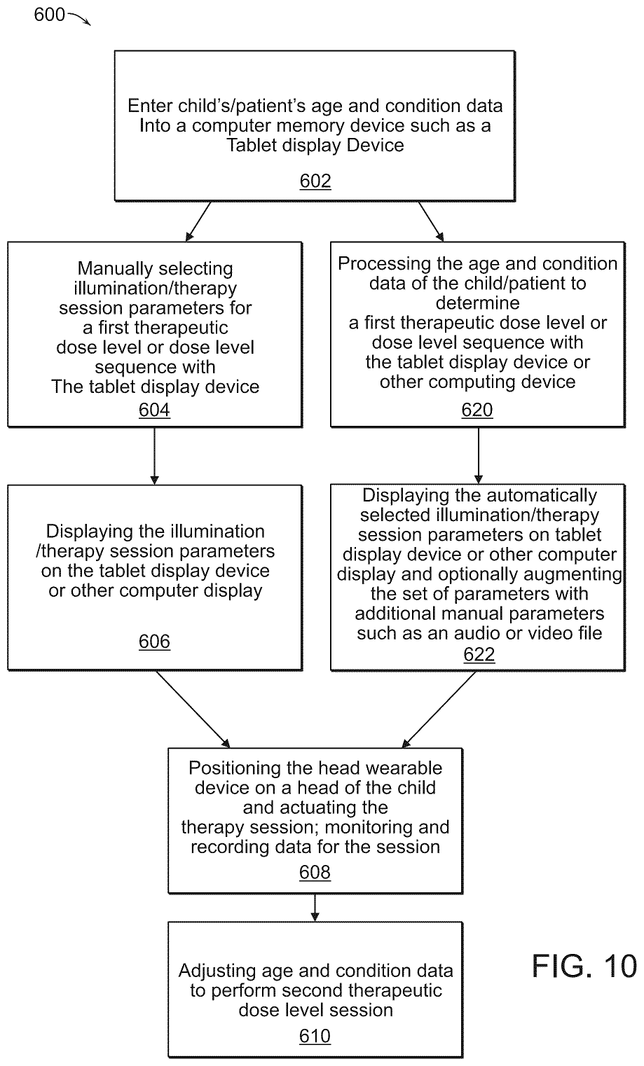

Thus, an operating module of the software can be programmed to retrieve fields of data or data files from a patient data entry module that can include patient information and other initial observations of parents or clinicians regarding a child's age, condition, medical history including medications that may impact a further diagnostic or therapeutic program. FIG. 10 illustrates a process flow diagram for a method 600 of selecting and optimizing parameters over multiple therapeutic sessions including manual and automated selection tracks. Initially, patient data related to a child or adult patient (such as age or condition) can be entered by a user into a memory of a computing device (step 602). For example, data can be entered by a user through the GUI 160 of the remote computing device 150 (such as a tablet computing device) and stored in the memory 156 as described previously in relation to FIG. 4. The method 600 can then follow one of two tracks. In one embodiment, the user can manually select illumination or therapy session parameters for a first therapeutic dose level or dose level sequence based upon the patient data (step 604). For example, the user can manually select parameters from menu or other displays on the GUI 160 of the remote computing device 150. Then, the illumination and/or therapy session parameters (which may include user-selected parameters and other parameters whether automatically determined or set by default) can be displayed on the computer display (step 606). For example, the parameters can be displayed on the visual display device 152. The device can also be programmed to operate a linguistic and/or visual message therapy module that communicates auditory and/or visual messages to the patient during a therapy session.

In an alternative embodiment, the parameters can be set algorithmically or automatedly. The processor of the computing device can process the patient data (including, for example, age and condition data) to determine the first therapeutic dose level or dose level sequence (step 620). For example, the processor 155 of the remote computing device 150 can analyze and process the patient data. Then, the automatically selected illumination and therapy session parameters (as well as other session parameters) can be displayed on the display associated with the computing device (step 622). Optionally, the set of automatically selected parameters can be augmented in this step with additional manual parameters such as an audio or video file used as part of the therapeutic session.)

Whether the parameters are determined automatically or manually, the head wearable device can then be positioned on the head of patient (e.g., a child or adult) and the therapy session can be actuated based on the session parameters (step 608). Data related to the patient or device during the session can be monitored and recorded. Then, the patient data (e.g., age or condition data) can be adjusted to optimize session parameters for future (i.e., second, third, or more) therapeutic sessions (step 610).

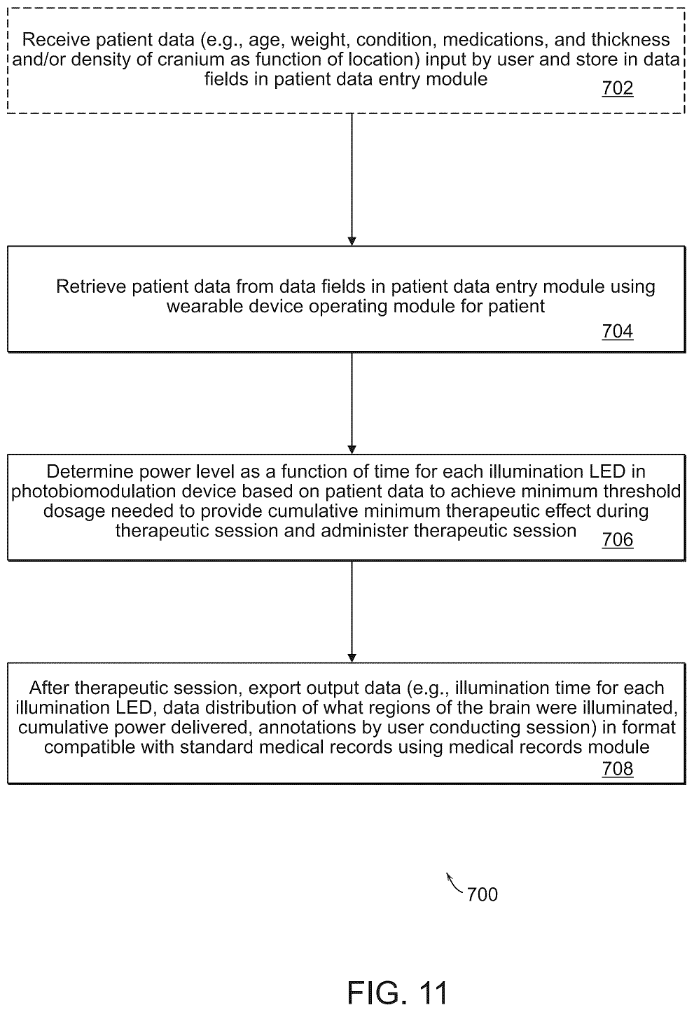

FIG. 11 illustrates a process flow diagram for a method 700 for administering a therapeutic session to a patient in accordance with various embodiments described herein. As an optional first step, patient data can be input by a user to a computing device and stored in data fields in a patient data entry module resident in the computing device or a server device (step 702). Relevant patient data entered in this step can include patient age, weight, physical or mental condition, medication history or regimen, and a data map of cranial thickness or density as a function of location on the patient's cranium. For example, the patient data entry module can reside in the memory 156 of the remote computing device 150, and patient data can be entered using the GUI 160 such as by using a keyboard, mouse, or multi-point touch interface 420. This step may be considered optional as the patient data for a particular patient may already be resident in patient data entry module (e.g., the data may have been entered during previous sessions and need not be re-entered). The patient data is then retrieved from the data fields in the patient data entry module using the wearable device operating module (step 704). The wearable device operating module can determine a power level as a function of time for each illumination LED 115a-115e in the array of the photobiomodulation device 110 based on the patient data to achieve the minimum therapeutic effect during the therapeutic session. Once the power levels are determined, the therapeutic session can be administered to the patient (step 706).

After concluding the therapeutic session, output data can be exported in a format compatible with standard medical records using a medical records module (step 708). Output data can include the illumination time and/or power for each individual illumination LED, a data distribution of which regions of the brain were illuminated, the cumulative power delivered, or annotations from a user conducting the session such as a medical professional. The data can be time-course data including time stamps that record when observations or other data events occurred within the therapeutic session.

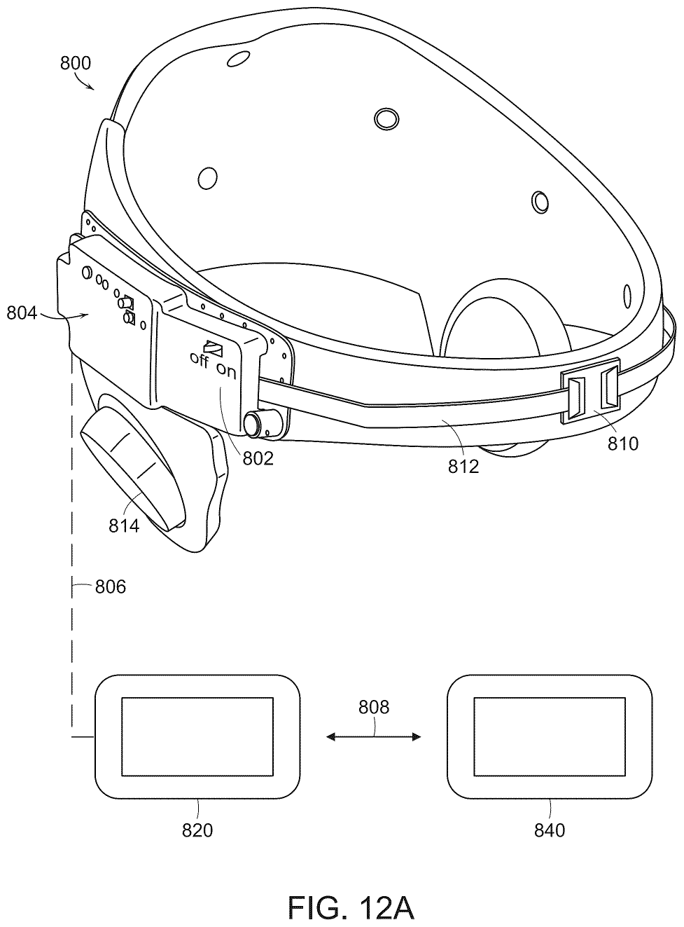

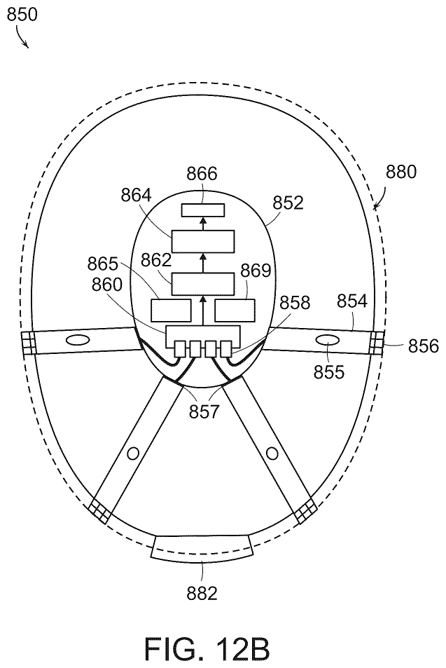

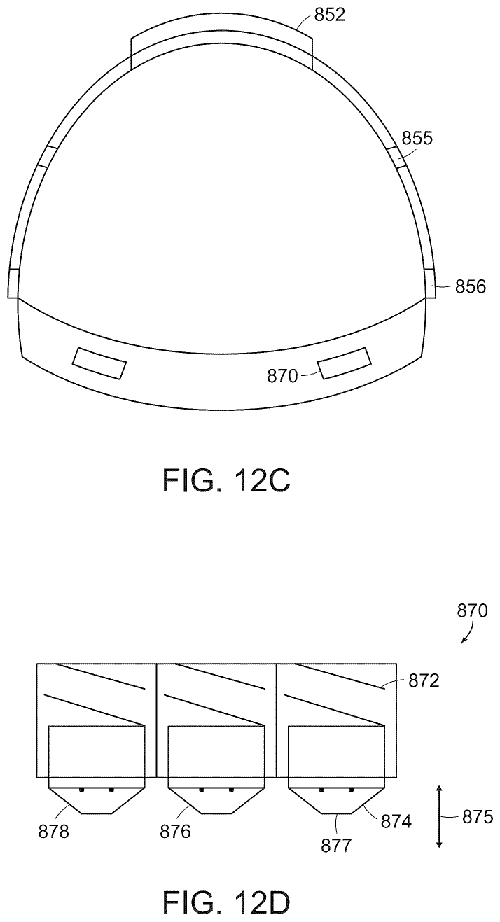

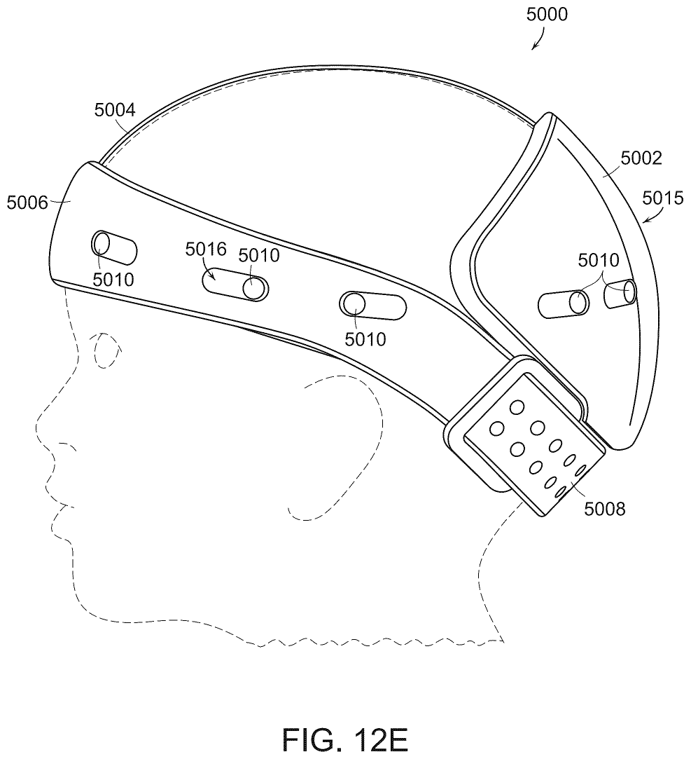

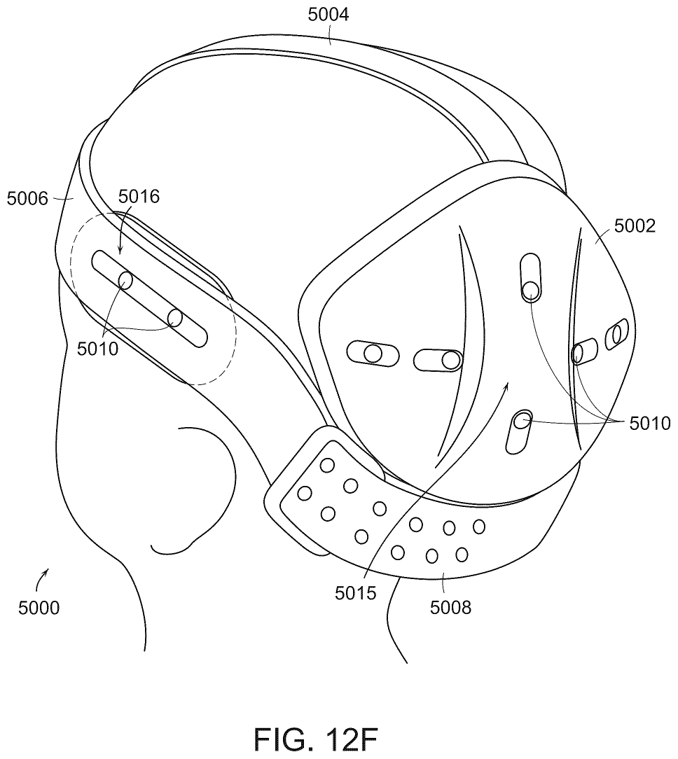

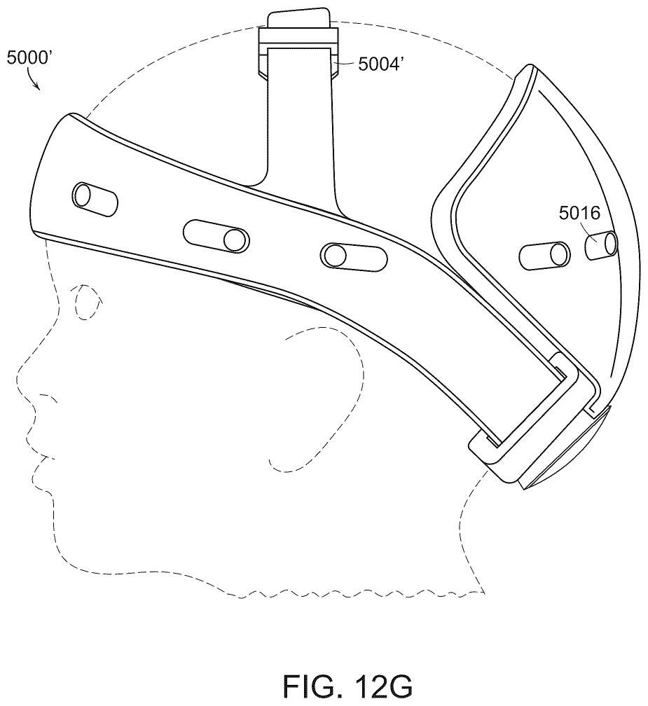

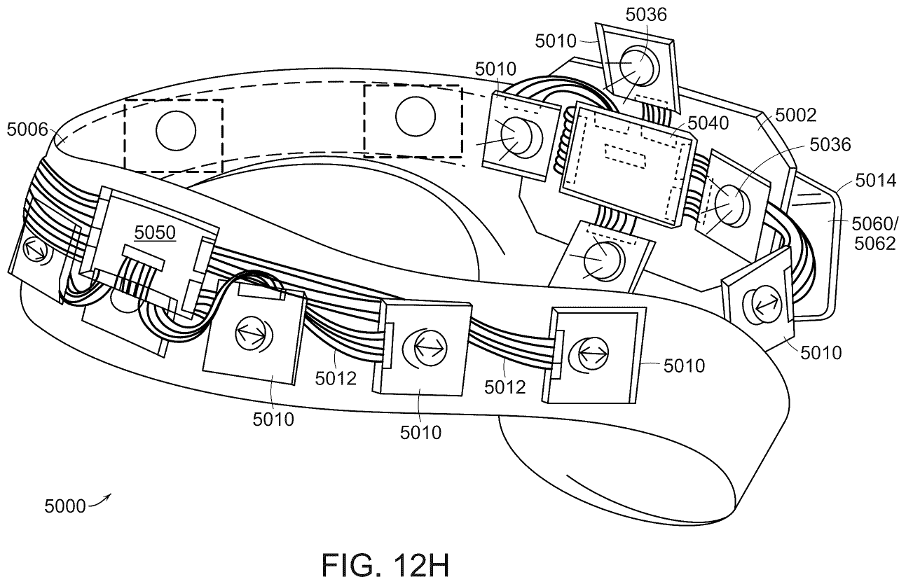

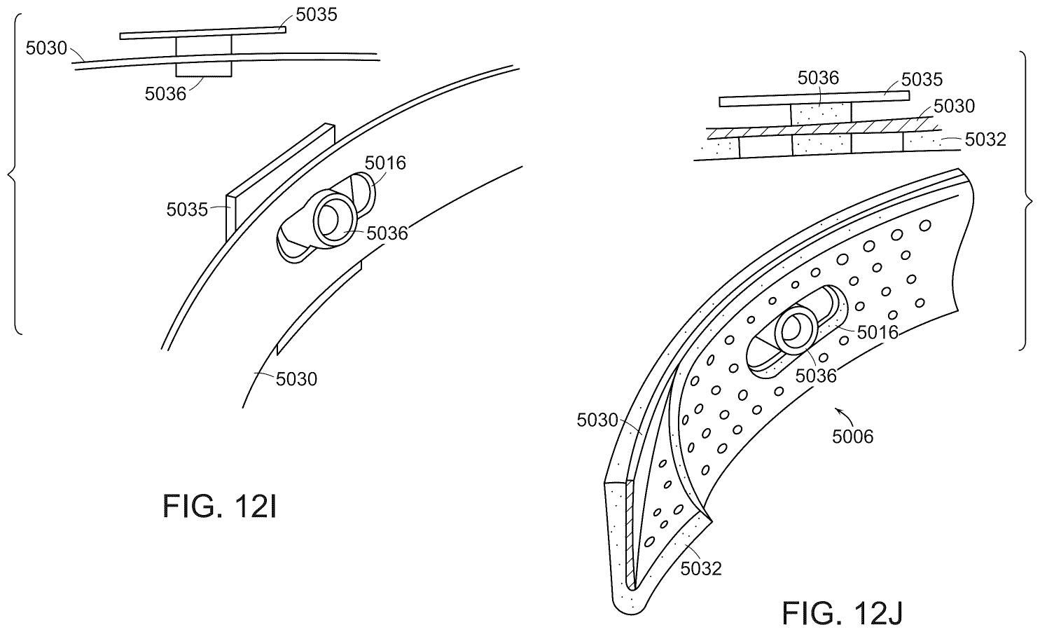

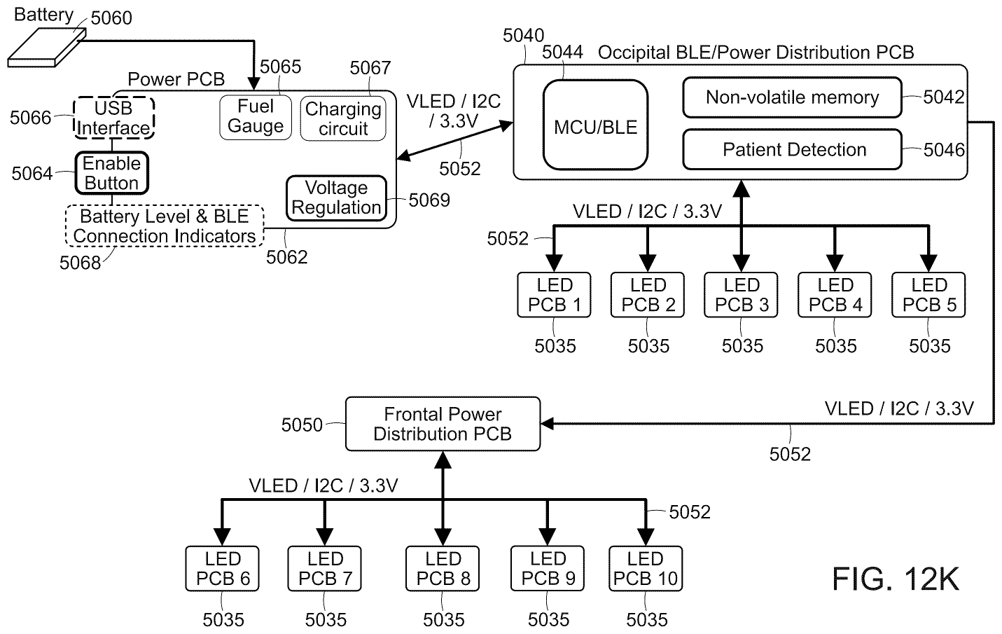

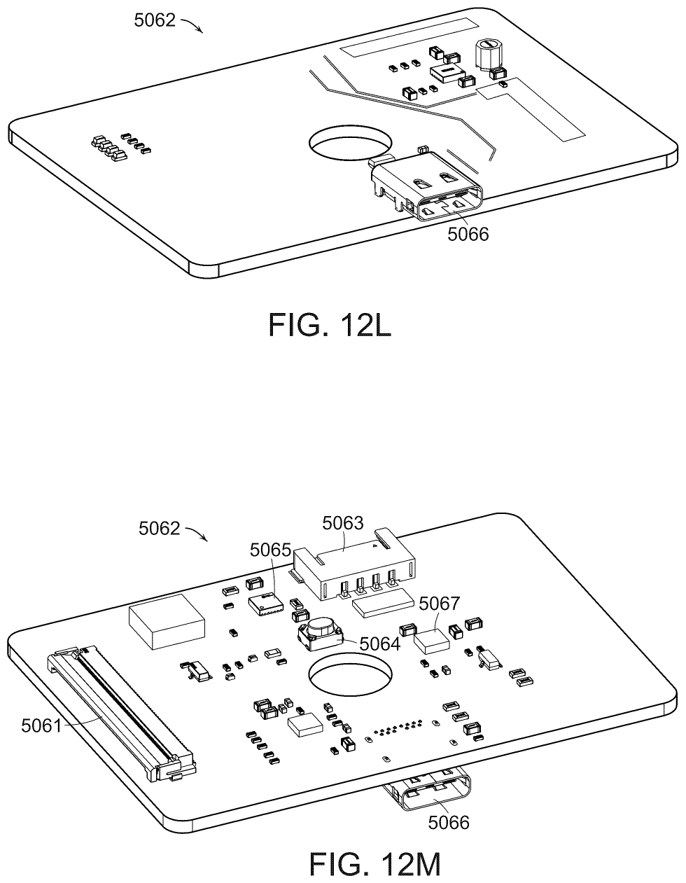

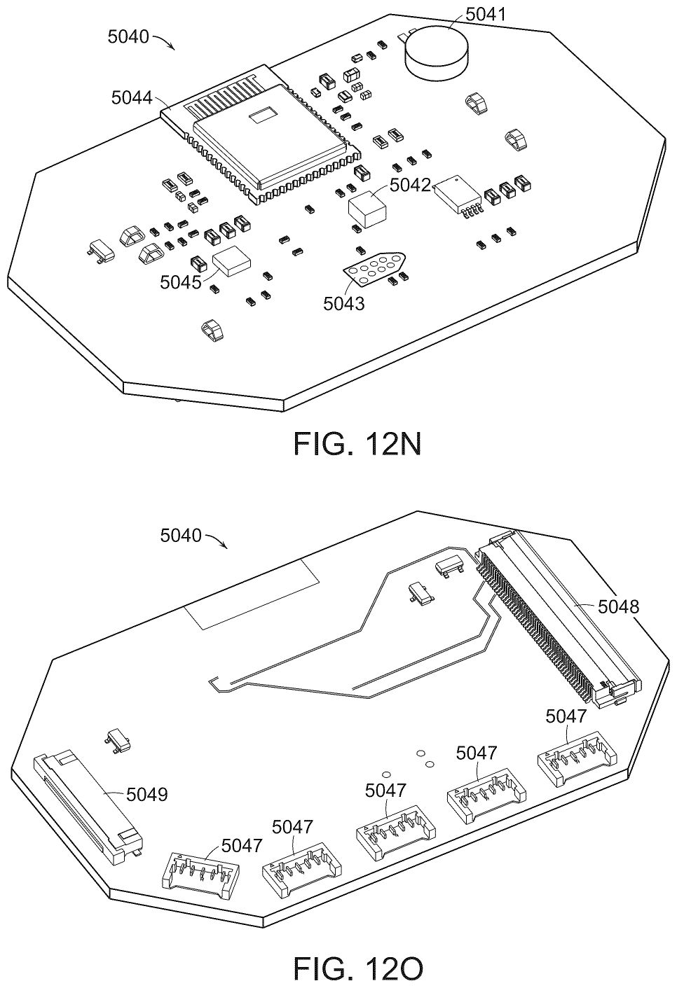

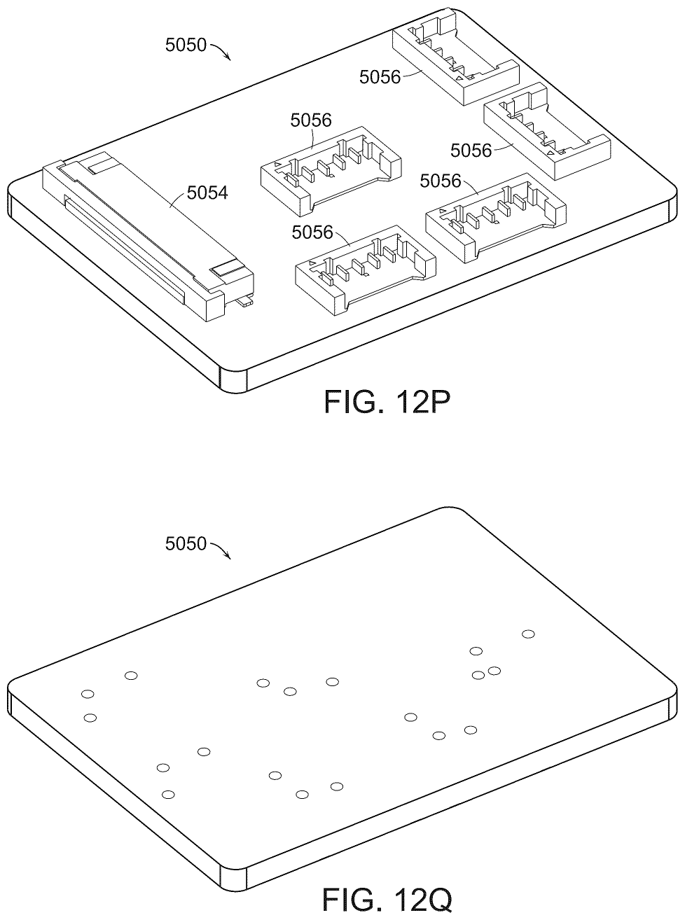

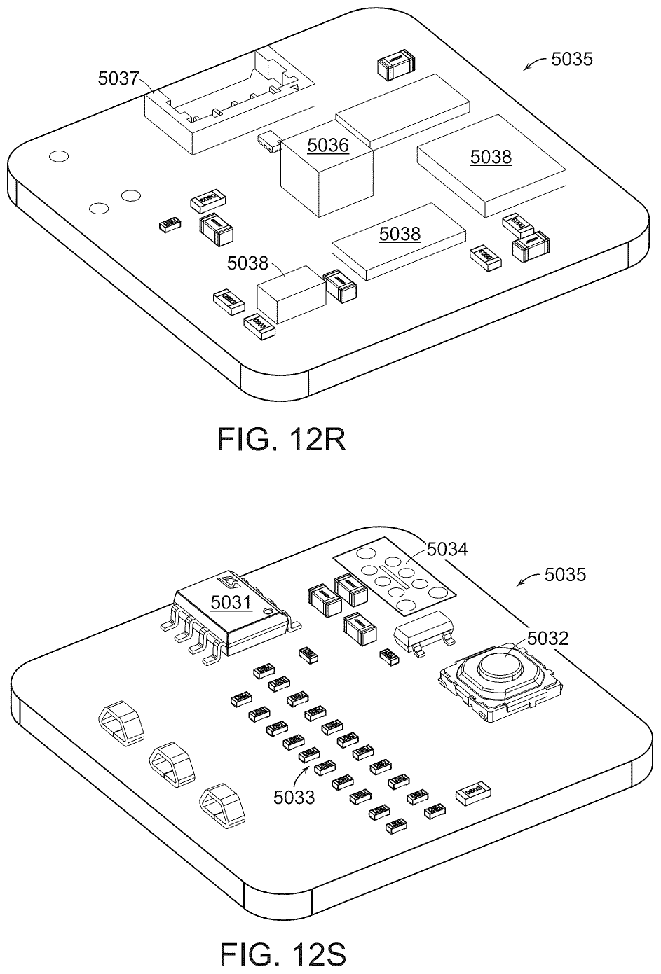

Shown in FIG. 12A is a further implementation in which the head wearable device 800 has light emitting devices 810 at spaced locations around the head of the patient connected by a cable 812 to a circuit housing having a first portion with an on/off switch 802 and a second portion with one or more control buttons or actuators 804 to manually select operating modes of the device as described herein. Headphone speakers and/or microphones 814 can be mounted to the head worn device 800 or speakers/microphones can alternatively be within a first tablet 820 that can be used by the patient during a therapy session. The first tablet or mobile phone 820 can be connected by wire or cable 806 to device 800 and can emit sounds or auditory signals for improving linguistic skills of the patient as described herein. The display on the first tablet can also be used to display images or video to the patient during the therapy session. A second tablet or mobile phone 840 can also communicate with the head worn device 800 and/or the first tablet by a cable or wireless connection 808. Tablet 840 can be used by an operating user to control operation of one or both of the head worn device 800 and first tablet 820, before, during or after a therapy session. For example, if an EEG sensor is used during a therapy session, this can serve to monitor the procedure or calibrate the power level to be used on a particular patient to establish the minimum level therapeutic dose, and optionally to also set a maximum dose for each period of illumination during the session, and further optionally to select which regions of the brain of the patient are to be illuminated during a session. The first tablet may be programmed only to provide the auditory and/or visual components to the patient, whereas the second tablet can be programmed solely for use by the operator or clinician to manage the therapy provided to one or more patients in separate sessions. The tablet used to manage patient data can also be connected by wired or wireless connection directly to an external EEG processing station 156 that receives wireless transmission of digitized EEG signals from the headset.

Shown in FIG. 12B is a top view of a headset 850 that incorporates an EEG electrode array including EEG electrodes 855, 856 located at different locations around the head of the patient. As described in further detail below such an EEG sensor array can be integrated with a light emitter array positioned around the head of the patient at different separate locations, or partially or entirely collocated with the EEG electrodes. The separation between light emitters and EEG electrodes can be adjusted depending on the treatment protocol for different neurological disorders as described herein. Light emitters and/or electrodes can be mounted on bands 854 that extend towards an upper housing or top portion 852 which can have a crown shaped bottom surface that can conform to the top of a user's head to help stabilize the housing 852 which preferably has a low profile shape with a light weight. The bands 854 can extend to a circumferential portion of the headset 880 extending around the user's head such as depicted in FIG. 12A and other figures shown and described herein. The bands 854 or tubes containing the necessary wiring for EEG electrodes and/or light emitters can be situated on all sides of the user's head so as to enable placement of light emitters and/or EEG electrodes as required for a specific application. Between 8-64 or more EEG electrodes can be mounted on the headset along with the same or a different number of light emitters as described herein. The tubes or bands 854 can also extend from the rear electronics module 882 for embodiments in which there is no housing 852 on the top of the patient's head such as depicted in connection with FIGS. 2A-3 and 9-12, in which case the EEG circuitry can be integrated into module 882. The tubes or bands can include connectors 857 at one end so that they can be easily removed and replaced. Such a system can thereby incorporate disposable components thereby allowing the electronics module to be reused with other patients without loss of sterile conditions. In a further embodiment, the housing can be configured to include circuitry for detecting EEG signals wherein wiring from the EEG electrodes 855, 856 is amplified with amplifiers 858 for each channel followed by analog to digital converter 860 for each channel, processing of the digital signals with processor 862 that can multiplex the signals for transmission by wireless transmitter 864 and antenna 866 that communicates with an external transceiver as described herein. A power source such as a battery 869 and an impedence excitation source 865 can also be located in the housing 852. The circuitry and one or more power sources in housing 852 can also be located in a second circuit housing 882 situated on the back of the patient's head. The housing 882 can also include circuitry, power and control operations for the light emitter system as previously described herein. A further embodiment can employ a battery situated in the rear housing to power both the circuitry in the top housing 852 and the rear housing 882. In a further embodiment, the circuitry in both housings can utilized a single processing unit to manage digital signals for both digital circuits. Such a control processor can be configured to control the light emitters, and the sensed EEG digital signals for external transmission. The transmitter 864 and antenna 866 can thereby operate as a transceiver to manage receipt of control signals to control operation of the light emitters and also control transmission of digitized EEG data. The EEG and light emitter control and processing functions can be performed by one or more processors. For applications requiring a larger number of EEG electrodes and/or light emitters one or more control and processing functions can be performed by a field programmable gate array (FPGA) or by an application specific integrated circuit (ASIC) configured to process the larger number of channels at faster speeds and at lower power levels. Such a configuration can further reduce the size and weight to accommodate use by pediatric patients. If a larger number of light emitters and/or EEG sensors is required, the electronic components required to operate the integrated system can also be mounted on a single flexible circuit board extending from the rear housing to the top housing within a single flexible sleeve. Note that the bands 854 can comprise flexible or semi-rigid plastic components. The bands can comprise tubes or prongs in which the wiring for the EEG electrodes or light emitters can extend. The light emitters can comprise LEDs or laser diodes, for example, that can be mounted in the ends of the tubes that are oriented to direct light through the cranium. A lens can be attached to the distal exit aperture of the light emitter to define the focal region of tissue within the cranium. Where an array of light emitters is used, the distal lenses can provide overlapping illumination volumes of tissue. The tubes can comprise polyethylene or polypropylene materials that can be sterilized or replaced after a single use. As seen in FIG. 12D, the EEG electrodes and/or light emitters 874, 876, 878 can each be spring loaded with springs 872 to cause the contact surfaces to press against the scalp. Thus, the tissue contact surfaces 877 can move relative to the housing along axis 875. Arrays of two or more EEG electrodes, light sensors and/or light emitters can be housed 870 at well-defined separation distances to provide repeatable measurements. Each housing 870 can be situated on a band at selected locations on the head so as to precisely locate the sensors and light emitters as described herein to transmit and receive signals through the cranium and also through the lambdoid suture and/or the squamosal suture. In pediatric patients these small lines between cranial plates have less density and are thus more transmissive of red and infrared light signals for photobiomodulation as described herein. X-ray images of these sutures lines can be obtained for each patient in which it is desirable to direct illuminating light through one or more suture locations. This enables precise positioning of the light emitters relative to the suture lines. In such applications, the headset must be properly secured to the head to align the light emitters to the suture lines for the therapeutic period.

FIG. 12E illustrates a side view of a head wearable device 5000 in accordance with some embodiments described herein. The head wearable device 5000 includes a head mounting frame or headband 5006 connected to an occipital mount 5002. The frame or headband 5006 can comprise a length of material that surrounds the head of the user wherein light sources are mounted to direct light inward through the cranium. The head mounted material has an inner surface and an outer surface on which circuit components can be mounted. The headband 5006 can include a size adjustment mechanism 5008. Alternatively, the optoelectronic circuit components described herein can be mounted on a flexible head wearable fabric with sufficient elasticity to be worn on different head sizes, but exert sufficient tension to cause the light emitting surface of the LEDs to come into contact with the scalp as previously described. Such features are described previously in the present application and can be adapted for this preferred circuit design. Note that this system can also include communication, user interface, audio and visual systems previously described generally in the present application. LED modules 5010 are mounted at locations on the headband 5006 and the occipital mount 5002 to illuminate different portions of a user's cranium with therapeutic light. The head wearable device 5000 can include a head strap or band 5004 that connects a frontal portion of the headband 5006 to the occipital mount 5002 at the back of the user's head.

The head wearable device 5000 is formed at least partially of a soft material with airy open spaces in some embodiments. In some embodiments, a surface of the headband 5006 is formed of a non-porous material to improve sterilizability and cleanability. In some embodiments, the material can include one or more of low-density polyethylene (LDPE), silicone, or ethylene-vinyl acetate (EVA) closed-cell foam. In some embodiments, the headband 5006 can include light, pastel, or bright colors that appeal to children for pediatric therapy applications.

The size adjustment mechanism 5008 can enable adjustment of the headband 5004 for comfort and/or to improve contact or coupling between the user's scalp and the LED modules 5010. The size adjustment mechanism 5008 can include a band or strap that tightens against a patient's skull or tightens below the occipital bone of the skull. In some embodiments, the adjustment mechanism can include a fastener such as a hook-and-loop fastener. For example, the headband 5006 can include two separated straps that fasten with the hook-and-loop fasteners or a single strap that passes through a retaining ring and doubles back upon itself so that hooks on the end of the strap can attach to a separate portion of the strap that includes loops. In some embodiments, the adjustment mechanism can include a snap closure wherein snaps or pegs in one portion of the headband 5006 connected with a variety of snaps or holes at different positions along the headband 5006. Similarly, the head strap 5004 can include a size adjustment mechanism to enable sizing adjustments of the head strap 5004 to improve comfort for a user with a given head size. The same size adjustment mechanisms 5008 described herein for the headband 5006 can be employed in the size adjustment mechanism for the head strap 5004.

FIG. 12F illustrates a rear view of the head wearable device 5000 illustrating the occipital mount 5002. The occipital mount 5002 can include one or more LED modules 5010. The LED modules 5010 can be spaced in a pattern in some embodiments such as a cross pattern or a polygonal patterns such as square-shaped or diamond-shaped. In some embodiments, the LED modules 5010 can be positionable at different positions on the headband 5006 or occipital mount 5002. For example, headband 5006 or occipital mount 5002 can include multiple receptacles at different locations so that the LED modules 5010 can be moved to different receptacles as needed. In some embodiments, the headband 5006 or occipital mount 5002 can include racetracks or slots 5016 that allow one or more of the LED modules 5010 to translate or slide in one or more directions to improve positioning of the LED modules 5010.

In some embodiments, the occipital mount 5002 can include an electronics housing 5014 to accommodate a battery or other electronics or power sources to power elements of the head mounted device 5000 such as the LED modules 5010.

FIG. 12G illustrates an alternative embodiment of the head wearable device 5000′ with different placement of the head strap 5004′ in accordance with some embodiments described herein. The head wearable device 5000′ is substantially identical to the head wearable device 5000 described above except that the head strap 5004′ extends from one lateral side of the headband 5006 to the other lateral side of the headband 5006. This differs from head strap 5004 as shown in FIGS. 12E-F that extends from the occipital mount 5002 forward to a portion of the headband 5006 adjacent to the patient's forehead. In some embodiments, the head strap 5004, 5004′ is omitted from the head wearable device 5000, 5000′ entirely.

FIG. 12H illustrates the head wearable device 5000 according to various embodiments described herein. This figure illustrates a patient-contacting surface of the occipital mount 5002 to show arrangement of LED modules 5010 and mounting of the occipital power distribution printed circuit board (PCB) 5040. In this embodiment, the occipital power distribution PCB 5040 controls power distribution to a group of five LED modules 5010 arranged as a group in the occipital region of the patient's brain. A separate frontal power distribution PCB 5050 is positioned at the forehead region of the headband 5006 and powers a group of five LED modules 5010. Each group of LED modules 5010 is connected to a respective PCB 5050 by connection wires 5012. In some embodiments, the connection wires 5012 pass directly from the respective PCB 5040, 5050 to the corresponding LED module 5010 as opposed to passing serially through multiple LED modules 5010. In this arrangement, direct powering and addressing of each LED module 5010 by the PCB 5040, 5050 is possible as there is no daisy chaining. This enables consistent power delivery to all LEDs on the head mounted device 5000.

FIGS. 12I and 12J illustrate placement of an LED module in a multi-material headband 5006 in accordance with some embodiments described herein. The headband 5006 can include a relatively stiffer core 5030 surrounded by a relatively softer foam liner 5032 in some embodiments. The core 5030 can retain an LED 5036 of an LED module 5010 in a stable position within an opening 5016 (such as an oval or racetrack opening) due to friction fitting between the LED 5036 and the core 5030. The foam liner 5032 can surround the stiffer core 5030 to provide a comfortable surface against the patient's head. As shown in the perspective and end views of FIG. 12I, the LED module 5010 can include the LED 5036 and LED PCB 5035, which is described in greater detail below. The LED 5036 projects from the LED PCB 5035 and extends through the core 5030. As shown in FIG. 12J, a front surface of the LED 5036 can be flush with the surface of the foam layer 5032 that contacts the patient so that the LED is placed as close as possible to the patient's scalp without projecting outward to form a painful pressure point.

FIG. 12K schematically illustrates the electrical connections between elements of the head wearable device in accordance with several embodiments described herein. The electrical system of the head wearable device 5000 includes a battery 5060, the power PCB 5062, the occipital power distribution PCB 5040 (sometimes abbreviated as the occipital PCB), and the frontal power distribution PCB 5050 (sometimes abbreviated as the frontal PCB). The power PCB 5062 is connected to the occipital PCB 5040 via a cable 5052. The cable 5052 can carry signals for multiple operations or functionalities simultaneously. In some cases, the cable 5052 can carry signals at different voltages. In various embodiments, the cable 5052 can transfer power to the LED PCBs 5035 at the appropriate voltage V_{LED}, can carry voltage for logic circuits such as TTL at 5 V or 3.3V, or carry voltage for inter-integrated circuit (I₂C) bus or Pulse-Width Modulation (PWM) Limit applications. Similar cables 5052 with similar functionalities connect the occipital PCB 5040 to the frontal PCB 5050; connect the occipital PCB 5040 to LED PCBs 5036; and connect the frontal PCB 5050 to LED PCBs 5036.

The power PCB 5062 can include a power gauge integrated circuit (IC) 5065, a charging circuit 5067, a voltage regulation module 5069, a USB interface 5066, an enable button 5064, and battery level and BlueTooth® low energy (BLE) connection indicators 5063. The power gauge IC 5065 can monitor the voltage level of the battery 5060 to determine the remaining energy (power) in the battery 5060. The battery level indicators 5068 can indicate visually to the user the level of remaining energy in the battery 5060 as measured by the power gauge IC 5065. The charging circuit 5067 enables wireless charging of the battery 5060 using inductive charging techniques such as those that conform to the Qi® wireless charging standard or other charging standards. The voltage regulation module 5069 can regulate the output voltage provided on the cable 5052 to the other components. The enable button 5064 can include a mechanical or electrical switch operable by the user to turn on or off the electrical systems of the head mounted device 5000. The USB interface 5066 enables wired charging of the battery 5060 and/or provides an interface for reprogramming (e.g., flashing) or debugging components of the power PCB or connected PCBs. The USB interface 5066 can also be used for transfer of data such as usage statistics (e.g., recorded or sensed power levels, up-time or down-time, or error statuses).

The occipital PCB 5040 includes a processor such as a microcontroller unit (MCU) and BlueTooth® low energy (BLE) module 5044, non-volatile memory 5042, and a patient detection module 5046. The MCU/BLE module 5044 can operate as a central control circuit that controls power output to each individual LED PCB 5054. The MCU/BLE module 5044 enables communication with external devices using the BLE protocol. For example, an external device such as a computer, tablet, or smartphone operated by the user can wirelessly send instructions to the MCU/BLE control module 5044 to adjust individual LEDs to different power settings over time according to a therapeutic program. The memory 5042 can include one or more of logical address information or instructions to control the LED PCBs 5154. The patient detection module 5046 can detect whether or not the head mounted device 5000 is being worn by the patient. If the patient detection module 5046 having a contact sensor detects that the head mounted device 5000 is not being worn by a patient, it can send signals to the controller 5044 or the power PCB 5062 to disable power to the LEDs to prevent light output. This automatic shutoff when the patient is not present can conserve power in the battery 5060 and provide safety by preventing illumination from being turned around when the light could accidentally enter a patient's eyes, for example. This is especially important in pediatric applications where a child may inadvertently remove the head mounted device 5000 and could accidentally aim the light at their eyes if it were not automatically shut off. In some embodiments, the patient detection module 5046 can operate by employing optical sensors that detect whether LED light is being reflected by a very close object. Alternatively, the patient detection module 5046 can use an accelerometer or inertial sensor system to determine the orientation of the head mounted device 5000 and disable the device when the position is not consistent with placement on a head of a sitting or standing individual.

FIGS. 12L and 12M illustrate respective top and bottom views of the power PCB 5062 in accordance with some embodiments described herein. The USB interface port 5066 is located on a top side of the power PCB 5062. The power gauge IC 5065, charging circuit 5067, and enable button 5064 are located on a bottom side of the power PCB 5062. The power PCB 5062 can include a connector 5063 to connect the cable from the battery 5060. The power PCB 5062 also includes a connector 5061 to connect the cable 5052 to the occipital PCB 5040.

FIGS. 12N and 12O illustrate respective top and bottom views of the occipital PCB 5040 in accordance with some embodiments described herein. The occipital PCB 5040 can include a battery 5041 to power an on-board real-time clock (RTC) that clocks operations of the device, a motion sensor such as an accelerometer 5045 for headgear orientation detection, an electronically erasable programmable read-only memory (EEPROM) 5042 or other volatile and/or non-volatile memory, the control module 5044, and a BLE control module programming port 5043 on a top side of the PCB 5040. The one or more memory devices on the head mounted device can retain information such as any error messages recorded during a treatment session, The memory also stores the duration of each treatment session, the amount of light delivered to the patient during each session and can also record annotations to the record for each session such as symptoms, side effects or proposed improvements or notes regarding the session by an observer. Each recorded session can automatically transfer a treatment session record that can temporarily be stored in the memory until the next session. The BLE programming port 5043 enables debugging or reprogramming of the control module 5044. The EEPROM memory 5042 can also be accessed and erased through the port 5043 or through signals sent from the power PCB 5062 through port 5066. The memory 5042 can include instructions for controlling LEDs in a particular program or pattern. The system can operate a first plurality of light sources according to a first pattern and control a second plurality of light sources (LEDs) according to a second pattern. Different light sources can emit at different optical wavelengths and at different duty cycles for example. The circuitry can include one or more current level sensors or temperature sensors to control operation of the device. This can include closed loop control of the light sources, for example, to maintain optical output from each light source within 5-10 percent of the nominal output to treat a selected condition of the patient for the prescribed therapeutic period. It is also important to prevent operating temperatures of the device so as to prevent thermal injury to the patient. Thus, the head mounted device is configured to automatically shut off the light sources and/or power if such a condition is detected. The accelerometer 5045 can send signals to the patient detection module 5046 to help detect whether the system is in a ready state for being mounted on the patient's head. The accelerometer or other sensor (pressure sensor, light sensor, etc as described herein) can also be configured to sense a change in orientation of the head mounted device relative to the users' head and to transmit a signal to the control module to shut off the LEDs. The system can also operate a state machine that is regularly updated with operational data that can be automatically transmitted to an individual that is monitoring the therapeutic session. An alarm signal can also be sent to a remote user communicating that at least a portion of the system has been disrupted or changed so that remedial action can be taken to continue or stop the therapy session. The system clock can report the time elapsed, the time remaining or the time of disruption.

The occipital PCB 5040 includes individual connectors 5047 to connect cables 5052 to the LED PCBs 5035. The occipital PCB 5040 also includes a connector 5049 to connect cables 5052 to the frontal PCB 5046 and a connector 5046 to connect the cables 5052 to the power PCB 5062. Any of the connectors 5049, 5047, 5048 can include a flat-flex connector that allows low-profile and flexible connections to reduce the space taken by cables 5052 within the headband 5006 or occipital mount 5002.

FIGS. 12P and 12Q illustrate respective top and bottom views of the frontal PCB 5050 in accordance with some embodiments described herein. The frontal PCB 5050 includes individual connectors 5056 to each LED PCB 5035 in the frontal or forehead region of the headband 5006. The frontal PCB 5050 also includes a connector 5054 to the cables 5052 from the occipital PCB 5040. The connector 5054 can be a flat-flex connector. Note that the use of two or more power control circuits mounted on the same or separate circuit boards can be used to control different sets of light sources. This can provide greater control over the operating conditions of the two or more groups of light sources to maintain operation with nominal operating conditions. Thus, a first plurality of light sources can be operated by a first power control circuit and a second plurality of light sources can be controlled by a second power control circuit. The use of a constant current control circuit improves the safe operation of the system. This system enables operation of the system at 500 milliamps and at a duty cycle of 35%, for example. It is desirable to operate the system at a currents above 400 milliamps and a duty cycle of less than 45% to improve safety, efficacy and durability of the system. This design is scalable, so that a third power control circuit can be used to control a third plurality of light sources, etc. Thus, a design of the system for older children or adult use can integrate more light sources to illuminate larger areas of the brain. In such a system 15-20 or more LEDs can be integrated for optimal control for a battery operated system, or for applications in which a power cable can be used to provide power to the head mounted system.

FIGS. 12R and 12S illustrate respective top and bottom views of the LED PCB 5035 in accordance with some embodiments described herein. The LED PCB 5035 can include a second controller 5031 with LED current feedback, an LED driving circuit 5038, the LED 5036, a temperature sensor, and an LED unique identification (ID) circuit 5033. The LED PCB 5035 can also include a connector 5037 to connect via cable 5052 with either the occipital PCB 5040 or the frontal PCB 5050. The LED driving circuit 5038 can be an LED constant current circuitry that maintains the proper current output to drive the LED 5035. The temperature sensor can sense the temperature and send signals to the microcontroller 5031. The microcontroller 5031 can halt power to the LED 5036 if an over-temperature or overheating condition is detected. The LED unique ID circuit 5033 can include a resistor bank that is set differently for each LED PCB 5035 in the system. The occipital PCB 5040 or frontal PCB 5050 can use the LED unique ID circuit 5033 to identify each connected LED PCB 5035 upon connection or at a subsequent time. The LED PCB 5035 can include a microcontroller reset switch 5032 that is not operator accessible but can be used during initial setup or repair. The LED PCB 5035 also can include a controller programming port 5034 to enable debugging or reprogramming of the LED PCB 5035.

Photobiomodulation can be used to treat several ailments including Alzheimer's disease, post-traumatic stress disorder (“PTSD”), cognitive enhancement, cognitive impairment from trauma and/or injury, depression, anxiety, mood disorders, Parkinson's Disease, strokes, Global Ischema, and Autism Spectrum Disorder (“ASD”). In particular, these ailments can be treated with transcranial photobiomodulation, which involves targeted light energy to the brain. The devices associated with performing transcranial photobiomodulation are often applied over the head, such as in certain embodiments described herein. However many such devices, can be cumbersome and in particular, for especially sensitive patients (e.g., children with ASD), it can be difficult to comfortably apply the device for treatment over a meaningful duration of time without the patients attempting to shift or remove the device.

Aspects of the disclosed technology include devices for photobiomodulation, which can be used to treat various patients including ASD children and older adults. Consistent with the disclosed embodiments, the photobiomodulation device may be sized and shaped to fit inside the oral cavity of the human mouth. The photobiomodulation device may include one or more light emitting diode (LED) lights, which may be located in a center portion of the device. Further, the LED emitters may be positioned to point downwards or to other regions, such that light from the device affects blood vessels that flow within the body to regions of the brain. Preferred embodiments of can be used in conjunction with methods and devices that can illuminate blood vessels within the brain or that supply blood directly to the brain such as the internal carotid artery. Further, the LED light emitters may emit light at one or more wavelengths which can be red, infrared, and/or a combination of the two. The LED light emitters may have a material (e.g., latex, silicone, rubber, etc.) surrounding it that allows the light to penetrate tissue within the mouth yet is also difficult to chew. The surrounding material can comprise one or more lenses to couple the emitted light onto the tissue that contacts a surface of the device and wherein the tissue contains regions of vascular flow that is illuminated with the device. The photobiomodulation device may further include an extendable portion that protrudes outwards from the device in a longitudinal direction. In some examples, the extendable portion may include the LED light emitters. The photobiomodulation device may be shaped similarly or substantially similar to a pacifier, for example. Therefore, a wearer of the photobiomodulation device can bite down or suck on the extendable portion while it is inside the mouth. The photobiomodulation device may further include one or more processors, transceivers, or power sources (e.g., batteries). Preferred embodiments can also include a cavity to collect a sample of fluid from within the mouth for further testing and analysis, such as a saliva sample. A surface of the device, or the cavity, can optionally include a sensor to measure further characteristics of the tissue and/or the sample. The sensor can be electronically connected to circuitry for readout of sensor data during use. The sensor can include a light sensor such as a photodetector to measure light from the tissue and/or sample. The device can be configured to communicate with an external portable communication device as previously described herein to store patient data in a memory, and to further process and communicate data as described in the present application.

In some examples, the frequency and/or type of light emitted by the photobiomodulation device may be adjustable. Therefore, the photobiomodulation device may further include a controller that allows the user to adjust the frequency, illumination pattern and/or intensity of light. Also, in some examples, the photobiomodulation device may be paired to a user device (e.g., via Bluetooth®) that can send instructions to adjust the operating parameters of light emitted. In some examples, the position of the LED light emitter may be adjustable, i.e., the LED light emitters can be moved or scanned in another direction (e.g., left, right, up, or down).

Some implementations of the disclosed technology will be described more fully with reference to the accompanying drawing. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

FIGS. 13A-13U illustrate an alternative embodiment of a head wearable device. FIG. 13A illustrates a side view of a head wearable device 5100 in accordance with some embodiments described herein. The head wearable device 5100 includes a head mounting frame or headband 5106 connected to an occipital mount 5102. The frame or headband 5106 can comprise a length of material that surrounds the head of the user wherein light sources are mounted to direct light inward through the cranium. The head mounted material has an inner surface and an outer surface on which circuit components can be mounted. The headband 5106 can include a size adjustment mechanism 5108. Alternatively, the optoelectronic circuit components described herein can be mounted on a flexible head wearable fabric with sufficient elasticity to be worn on different head sizes, but exert sufficient tension to cause the light emitting surface of the LEDs to come into contact with the scalp as previously described. Such features are described previously in the present application and can be adapted for this preferred circuit design. Note that this system can also include communication, user interface, audio and visual systems previously described generally in the present application. LED modules 5110, 5110′ are mounted at locations on the headband 5106 and the occipital mount 5102 to illuminate different portions of a user's cranium with therapeutic light. The head wearable device 5100 can include a head strap or band 5104 that connects a frontal portion of the headband 5106 to the occipital mount 5102 at the back of the user's head.

The head wearable device 5100 is formed at least partially of a soft material with airy open spaces in some embodiments. In some embodiments, a surface of the headband 5106 is formed of a non-porous material to improve sterilizability and cleanability. In some embodiments, a surface of the headband 5106 is formed of a perforated material to improve heat dissipation and breathability. In some embodiments, the material can include one or more of low-density polyethylene (LDPE), silicone, or ethylene-vinyl acetate (EVA) closed-cell foam. In some embodiments the material can have cushion; for example, the material could be 2 mm in thickness. In some embodiments, the headband 5006 can include light, pastel, or bright colors that appeal to children for pediatric therapy applications.

The size adjustment mechanism 5108′ can enable adjustment of the headband 5104 for comfort and/or to improve contact or coupling between the user's scalp and the LED modules 5110, 5110′. The size adjustment mechanism 5108′ can include a band or strap that tightens against a patient's skull or tightens below the occipital bone of the skull. In some embodiments, the adjustment mechanism 5108, 5108′ can include a fastener such as a hook-and-loop fastener. For example, the headband 5106 can include two separated straps that fasten with the hook-and-loop fasteners or a single strap that passes through a retaining ring and doubles back upon itself so that hooks on the end of the strap can attach to a separate portion of the strap that includes loops. In some embodiments, the adjustment mechanism 5108 can include a snap closure wherein snaps or pegs in one portion of the headband 5106 connected with a variety of snaps or holes at different positions along the headband 5106. Similarly, the head strap 5104 can include a size adjustment mechanism 5108′ to enable sizing adjustments of the head strap 5104 to improve comfort for a user with a given head size. The same size adjustment mechanisms 5108 described herein for the headband 5106 can be employed in the size adjustment mechanism 5108′ for the head strap 5104.

FIG. 13B illustrates a rear view of the head wearable device 5100 illustrating the occipital mount 5102. The occipital mount 5102 can include an adjustment knob 5120 to enable size adjustments of the head band 5106 and/or the head strap 5104 as described above. The adjustment knob 5120 may be manually manipulated to adjust the size of the head band 5106 and head strap 5104. In some embodiments, the headband 5106 can be lengthened in the direction indicated by the arrow in the rotational direction indicated on the adjustment knob 5120. The occipital mount 5102 can include one or more LED modules. The LED modules can be spaced in a pattern in some embodiments such as a cross pattern or a polygonal patterns such as square-shaped or diamond-shaped. In some embodiments, the LED modules can be positionable at different positions on the headband 5106 or occipital mount 5102. For example, headband 5106 or occipital mount 5102 can include multiple receptacles at different locations so that the LED modules can be moved to different receptacles as needed. In some embodiments, the headband 5106 and the occipital mount 5102 can include receptacles at different locations so that the LED modules can be moved to different receptacles as needed. In some embodiments, the headband 5106 or occipital mount 5102 can include racetracks (not pictured) that allow the LED modules to translate or slide in one or more directions to improve positioning of the LED module. The LED modules, positioned on the headband 5106, can be enclosed within the material, such that, for example only the LED 5036 is exposed. In some embodiments the LED 5036 is flush with the material. In some embodiments, the LED 5036 is extends outside the material.

The occipital mount 5102 can also include circuitry, power and control operations for the light emitter system as previously described herein. In a further embodiment, the circuitry in the occipital mount 5102 can utilize a single processing unit to manage digital signals for the digital circuits. Such a control processor can be configured to control the light emitters, and the sensed EEG digital signals for external transmission. The EEG and light emitter control and processing functions can be performed by one or more processors. In some embodiments, the occipital mount 5102 can include an electronics housing to accommodate a battery (not pictured) or other electronics or power sources to power elements of the head mounted device 5100 such as the LED modules 5110. In some embodiments, the occipital mount 5102 can include a membrane control panel 5144. The membrane control panel 5144 can include an on/off button 5182, a Bluetooth connectivity 5184 indicator, an IR Therapy indicator 5186, and/or a battery fuel gauge 5188.

FIGS. 13C-13F illustrates exploded views of the occipital mount 5102. FIG. 1313E illustrate respective front and rear exploded views of the occipital mount 5102 in accordance with some embodiments described herein. The occipital mount 5102 is configured to communicate with an external computing device. In some embodiments this communication is achieved with a wireless connection. The communication can include user provided instructions of illumination parameters and an illumination period. The occipital mount 5102 can include a battery 5114 to power on the main circuit board 5178 and thereby the LED modules 5110, 5110′ wired to the main circuit board 5178. Some embodiments can includes a charging cord 5190 and a USB interface-charging port 5180 that enables a charging circuit to charge the battery 5114 which can be a 3.7 V lithium battery in this example. The circuitry can be mounted on a circuit board in which a processor, such as microcontroller, is connected to a battery gauge 5188, a Bluetooth connectivity 5184 indicator, an IR Therapy indicator 5186, power supply regulator, LED control field effect transistor and LED 5110. 5110′. The main circuit board is attached to a strap retainer 5126 and held in place with fasteners 5138. The strap retainer 5126 allows the adjustment mechanism 5108 to lock into place a maintain a certain circumference of the headband 5106. The strap retainer is secured to the proximal housing 5134, over the LED modules 5110′. In some embodiments, a majority of the occipital mount 5102 components are assembled to the proximal housing 5134. The LED modules 5110′ are attached to the proximal housing 5134. A fastener 5138 secures the center of the proximal housing to the strap retainer 5126. A cover 5146 is placed over the fastener 5138 to cushion the contact with the user's head. A closed cell foam pad 5128 can attach to the rear of the proximal housing 5134 and cover the remaining fasteners. Located on the front of the occipital mount 5102 is the membrane control panel 5144 and adjustment knob 5120. The adjustment knob 5120 can include a rigid knob 5122 with a pinion gear 5142 and a soft elastomer cover 5124 over the rigid knob 5122. Some embodiments can include a cam mechanism to couple to adjustment knob 5120 to the occipital mount 5102. Located on the underside of the occipital mount is the PS label 5148. FIG. 13F illustrates another exploded rear view of the occipital mount. Beneath the closed cell foam pad 5126 can be a molded pocket 5176 in which the momentary capacitive touch sensor 5174 is positioned in.

FIGS. 13G-I illustrates the head wearable device 5100 according to various embodiments described herein. This figure illustrates a patient-contacting surface of the occipital mount 5102 to show arrangement of LED modules or panels 5110. FIGS. 13H and 13I illustrates placement of an LED module in a multi-material headband 5106 in accordance with some embodiments described herein. In some embodiments, the head wearable device 5100 can include ten LED modules 5110 that can be parallel with respect to each other. The number of modules mounted on the head wearable device, or the number modules that are actually selected to illuminate different regions of the brain during a therapeutic period, can vary depending upon the age and the specific type of neurotherapy prescribed for each patient. Generally between 4 and 20 LED modules can be effective depending on the condition being treated. Older children and adults may require a larger number of modules delivering a larger dose during a treatment period, for example. The LED modules 5110, 5110′ are positioned throughout the head wearable device 5100 such that they illuminate the patient from a plurality of different angles. A second capacitive sensor 5168 can be located on the headband 5106 in addition to the momentary capacitive sensor 5174 located in the occipital mount 5102. In some embodiments, the headband 5106 can include a connector pod 5170. In some embodiments, wire management clips 5172 are included. The wire management clips 5172 may, for example, include channels to contain and organize wires 5192. As described above, in some embodiments, the headband 5106 or occipital mount 5102 can include tracks 5116. FIG. 13J illustrates the LED 5036 being adjusted within the tracks 5116 in the direction of the arrows. The tracks 5116 allow the LED modules 5110 to translate or slide in one or more directions to improve positioning of the LED module 5110. This is particularly important where the headband 5106 has been adjusted to fit the patient's head. To facilitate effective therapy treatment, the LED modules 5110 must be positioned over certain areas of the patient's brain. As the headband 5106 is adjusted to fit the patient's head, the LED modules 5110 may be positioned in unideal locations. The tracks 5116 allow the LED modules 5110 to be adjusted to improve positioning within the track. In an embodiment the track 5116 may support the LED in a left 5116a, central 5116b or right 5116c position. In one embodiment tracks 5116 can include three adjustment positions 5117, 5117′, 5117″ which may correspond to LED positions for a large, medium or small patient.

In some embodiments, the head wearable device 5100 can include an occipital power distribution PCB. In this embodiment, the occipital power distribution PCB controls power distribution to a group of five LED modules 5110 arranged as a group in the occipital region of the patient's brain. A separate frontal power distribution PCB can be positioned at the forehead region of the headband 5106 and powers a group of five LED modules 5110. Each group of LED modules 5110 is connected to a respective PCB by connection wires 5012. In some embodiments, the connection wires 5192 pass directly from the respective PCB to the corresponding LED module 5110 as opposed to passing serially through multiple LED modules 5110. In this arrangement, direct powering and addressing of each LED module 5110 by the PCB is possible as there is no daisy chaining. This enables consistent power delivery to all LEDs on the head mounted device 5110. Furthermore, two or more power control circuits mounted on the same or separate circuit boards can be used to control different sets of light sources. This can provide greater control over the operating conditions of the two or more groups of light sources, for example the LED modules 5110, 5110′, to maintain operation with nominal operating conditions. Thus, a first plurality of light sources can be operated by a first power control circuit and a second plurality of light sources can be controlled by a second power control circuit. The use of a constant current control circuit improves the safe operation of the system. The control circuit operates in response to programmed instructions. This operation enables the processor to execute a sequence of steps to activate certain LED modules 5110, 5110′ at a selected level of light to illuminate different regions of brain tissue of the patient. The selected level of light may be manually selected by the user with a user interface in communication with the wearable head device 5100.

FIG. 13K illustrates the layers of material of the headband or frame 5106 and the placement of an LED module in a multi-material headband 5106 in accordance with some embodiments described herein. The headband 5106 can include a loop panel that attaches to the fold panel 5195 that can comprise 2 mm thick neoprene, for example, wherein hook panels, 5198, 5199 attach to portions of fold panel 5195, and sew panel 5194 can comprise a stitched material 2 mm thick on the inside surface of the frame, in some embodiments. In this embodiment, the stiffer core are loop panels 5196, and 5167. A loop panel 5167 can include cutouts or openings in portions of the frame to accommodate the LED 5036 of the LED modules or LED circuit board panels 5110. A hook panel 5199 similarly includes cutouts or openings to accommodate the LED 5036 of the LED modules 5110. The fold panel 5195 also includes cutouts or openings 5116. The hook panel 5199 with cutouts adheres to the headband 5106. The loop panel 5167 with cutouts adheres to the section of the fold panel 5195 that includes cutouts or openings 5116. The hook panel 5198 adheres to another section of the fold panel 5195. The loop panel 5196 adheres to the inner facing side of the sew panel 5194. The surrounding or sew panel 5194 can encapsulate the multi-material elements.

The interior material can retain an LED 5036 of an LED module 5110 in a stable position within an opening 5116 (such as an oval or track opening) due to friction fitting between the LED 5036 and the core 5196, 5197. In some embodiments, the foam liner 5195 can surround the core 5196, 5197 to provide a comfortable surface against the patient's head. The LED 5036 projects from the LED module 5110 and extends through the multi-material. A front surface of the LED 5036 can be flush with the surface of the foam layer 5195 that contacts the patient so that the LED emission surface is placed as close as possible to the patient's scalp without projecting outward to form a painful pressure point. FIG. 13L illustrates the headband 5106 being adjusted. The headband 5106 can be lengthened or shortened with position adjustment of the LEDs being illustrated by the arrows. For example, the headband may be sized for a small pediatric head 1300a, a medium pediatric head 1300b, a medium adult head 1300c or a large adult head 1300d.

FIGS. 13M-O illustrates the LED modules 5110 attached to the headband 5106. FIG. 13M illustrates an interior side perspective of the LED module 5110. The LED module 5110 can include an LED bezel 5150, LED housing 5152, LED circuit board 5154, an IP membrane film 5156 that is a porous material such as a fabric or polymer barrier allowing air to pass through and heat to escape, and an aluminum cover 5158 having apertures for heat transmission away from the patients head. In some embodiments, the distance from the LED to the edge of the LED bezel or emission cone 5150 can be 3.62 mm. In some embodiments, the distance from the LED to the edge of the LED bezel or emission cone 5150 can be 4 mm. In some embodiments, the distance to the edge of the LED bezel or emission cone 5150, including the diode, can be 6 mm. In some embodiments, the distance to the edge of the LED bezel 5150 or cone, through which light is directed onto the skin of the patient and through the portion of the cranium within the emission aperture of cone, including the diode, can be 6.4 mm. The cone can comprise an elastic polymer material to improve the comfort for the patient as well as efficient optical coupling. Thus, a range of the distance from the LED emitting surface to the outer surface of the emission cone is preferably between 3-7 mm. In some embodiments, the light emitted from the LED 5036 produces a 40 degree light cone in one example with the range can be from 25 degrees to 50 degrees for the cone of the emitted light. The circuit board 5154 can have a pad 5156 such as a fabric or polymer that transfers heat from the circuit board through the apertures of metal cover 5158. A film can be formed on the outside of cover 5158 in prevent ingress of fluids into the module or panel assembly (5152,5154, 5156, 5158) while also facilitating thermal transmission. FIG. 13N illustrates the first side of the LED housing front panel 5152 which can comprise a molded polymer with a central opening for the LED to be mounted on for transmission of light through the opening 5160. The LED housing front panel 5152 can include heat dissipating castellations around the opening 5160.

The LED circuit board 5154 can include a controller 5151 on the bottom or second side of the circuit board (see FIGS. 13T and 13U) with LED current feedback, an LED driving circuit 5153 on the top or first side of the circuit board, the LED 5154 (shown in FIG. 13T), a temperature sensor 5135 (thermistor circuit), and an LED unique identification (ID) circuit includes the controller 5151 to enable recording of the LED emission data for each LED during a treatment period. The LED circuit board 5154 can also include a connector to connect via cable with either the occipital PCB or the frontal PCB. The LED driving circuit can be an LED constant current circuit 5153 that maintains the proper current output to drive the LED 5036. The temperature sensor 5135 can sense the temperature and send signals to the microcontroller. The microcontroller can halt power to the LED 5036 if an over-temperature or overheating condition is detected. The LED unique ID circuit can include a resistor bank that is set differently for each LED PCB in the system. The occipital PCB or frontal PCB can use the LED unique ID circuit to identify each connected LED PCB upon connection or at a subsequent time. The LED circuit board 5154 can include a microcontroller reset switch that is not operator accessible but can be used during initial setup or repair. The LED circuit 5154 also can include a controller programming port to enable debugging or reprogramming of the LED circuit board 5154.

FIGS. 13P-R illustrate secondary LED modules 5510′ attached to the occipital mount 5102. The secondary LED modules 5510′ are in accordance with some of the embodiments described herein. FIG. 13P illustrates an interior side perspective of the LED module 5110′. In some embodiments, the distance from the LED to the edge of the LED bezel 5150′ can be 7 mm. In some embodiments, the distance to the edge of the LED bezel 5150, including the diode, can be 9.4 mm. This LED module 5110′ includes a longer LED bezel mount 5150′, a curved LED housing 5162, and membrane film 5156. This LED module also includes lugs 5164 for securing the LED module 5110′ in the occipital mount 5102. FIG. 13S illustrates these LED modules 5110′ wired directly to the main circuit board 5178 via wires 5192 according to some embodiments.

FIG. 13V illustrates patient ergonomics and various head strap sizes in various embodiments taught herein. For example, anthropometrics/ergonomics are shown via a front 1301, side 1302 and back 1303 views of a patient's skull are depicted that correspond to those areas of the cranium wherein light is transmitted to illuminate those regions of the brain that are responsive to the therapeutic delivery off light as described herein for the treatment of neurological disorders, including autism as described herein. Also shown are views of a pediatric head strap for a 2 year old 1304 and 10 year old 1306a and 1306b as well as a view for an head strap suitable for an 18 year old patient 1308. As the circumferential size of the frame must be adjusted to enable a stable placement of the light sources relative to the specific areas of the brain for treatment, a two stage process for the adjustment of the position of the light sources relative to the cranium is advantageous. The first being the manual or motorized adjustment of the frame circumference. Thus, each individual patient can have the frame properly configured to match the patient's head circumference. In one embodiment, a manually adjusted actuator, such as a rotating element on a housing positioned on the back of the child's head is preferred. With this position, it is difficult for the child to see, reach or operate this feature, thereby preventing the unwanted interruption of treatment. The control panel can also be positioned in the housing at the back of the user's head to minimize access and avoid disruption of treatment.

FIG. 13W illustrates an exemplary control panel 1312 in various embodiments taught herein. The control panel includes indicator lights for power 1314, Bluetooth connectivity 1316, IR status 1318 and battery power level 1319.

FIG. 13X illustrates exemplary connector pod locations 1320 in various embodiments taught herein.

FIG. 13Y illustrates exemplary wiring channels 1322 in various embodiments taught herein. These channels contain and organize wires connecting the individual light emitting panels and sensors that reside on the frame.

FIG. 13Z illustrates exemplary rear LED PCB wiring in various embodiments taught herein. As shown wiring 1324 may be used to connect the five pods 1326 directly to the main PCB located within the rear housing.

FIG. 13AA illustrates exemplary capacitive touch sensor locations. For example, exemplary locations may include a first location 1330 on head strap 1332 and a second location 1334 on rear head pad 1336.

FIG. 13AB illustrates exemplary add-on LED locations in a housing in various embodiments taught herein. For example, housing 1340 may include two add-on locations 1342.

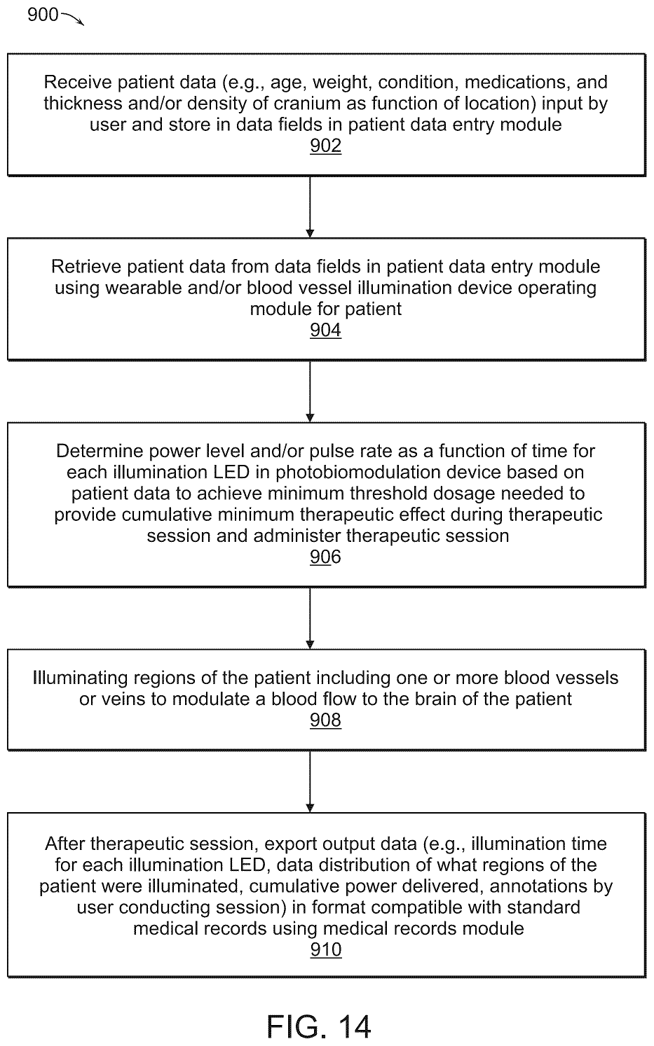

Shown in FIG. 14 is a process sequence 900 that can be implemented with a controller on the therapeutic device or in conjunction with an external controller as described herein. The user interface is configured to receive and store patient data 902. Certain data can be retrieved manually or automatically 904 so that parameters for a therapeutic session as implemented 906 on the PBM device. The device is actuating to illuminate vascular tissue of the patient 908 to thereby modulate blood flow within the body including the brain of the patient. This can be implemented in combination with transcranial illumination of brain tissue in selected patients, which can include transcranial illumination of blood vessels in proximity to brain tissue that is also receiving light. A record of the therapeutic session is than communicated 910 for storage and further analysis.

Further methods of the invention can include photobiomodulation of lymphatic vessels to improve drainage to treat neurological conditions. See, for example, the publication by Semyachkina-Glushkovskaya et al., “Photobiomodulation of lymphatic drainage and clearance; perspective strategy for augmentation of meningeal lymphatic functions”, Biomedical Optics Express, Vol. 11, No. 2, February 2020, the entire contents of which is incorporated herein by reference. By using PBM to augment the rate of drainage of lymphatic fluid from the brain there are improvements in transport of components that adversely impact neurological condition of the patient. Improved drainage of the lymphatic system has been shown to improve the condition of autistic patients. See Antonucci et al., “Manual Lymphatic Drainage in Autism Treatment”, Madridge Journal of Immunology, Vol. 3, Issue 1, December 2018, the entire contents of which is incorporated herein by reference. Thus, methods of treatment can include transcranial PBM of lymphatic channels in the brain. The LED array elements can be actuated to illuminate lymphatic channels at the energy densities described herein to perform therapeutic treatment of the patient. Imaging technologies including Optical Coherence Tomography (OCT) and ultrasound have been used to monitor lymphatic flow as well as blood flow and perfusion.

Methods for providing photobiomodulation may include determining the light frequency, location of the LED lights (e.g., blood vessels needing increased ATP), whether ATP production increased, and the overall effect of the treatments. Accordingly, based on the determined overall effect on the brain, the photobiomodulation device 1100 may be dynamically adjusted on a user-specific basis.

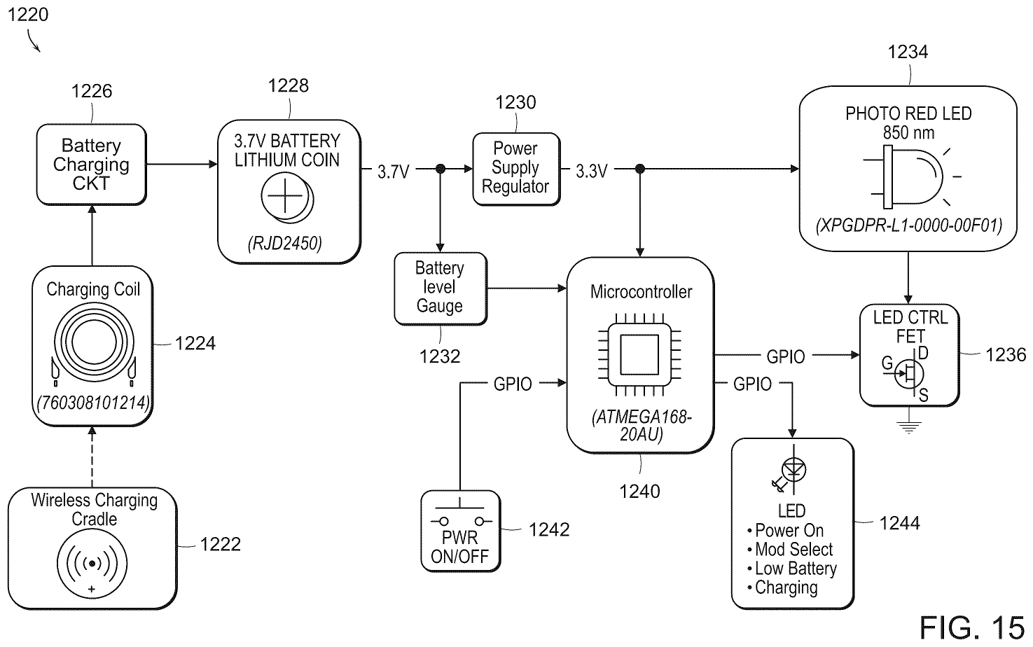

FIG. 15 illustrates an exemplary circuit 1220 for operating the PBM device. This embodiment can include a wireless charging element 1222 connected to a charging coil 1224 that enables charging circuit 1226 to charge the battery 1228 which can be a 3.7 V lithium battery in this example. The circuitry can be mounted on a circuit board in which a processor such as microcontroller 1240 is connected to a battery gauge 1232, power supply regulator 1230, LED control field effect transistor 1236 and LED 1234. In this example, an LED wavelength of 850 nm is shown but other wavelengths, or different wavelengths in the red and/or near infrared range can be used at different locations on the head mounted device as described herein. The device can include an on/off switch 1242 and an LED status indicator light 1244.

Fetal Alcohol Syndrome (FACS) results from a baby being exposed to alcohol during the neonatal stage of development. The fetal liver cannot metabolize alcohol (ethanol), so when alcohol enters the blood stream of the developing baby it interferes with the delivery of nutrition and oxygen to the developing organs. Therefore, it interferes with cell growth and proliferation. Specifically, ethanol in the developing baby's blood stream can result in permanent and irreversible brain damage. Neurological and behavioral symptoms often reflect the affected brain areas. The most common affected brain areas are the prefrontal cortex, which results in difficulties with focus, decision making and social interactions; the hippocampus can also be affected, which results in difficulties with forming memories; the cerebellum, which results in difficulties controlling movements; and also the corpus callosum, which affects overall brain function and results in mental retardation.

Brain imaging studies have specifically identified these areas (frontal lobe, corpus callosum, hippocampus and cerebellum) as being most likely to be affected by FACS. Other imaging studies showed that FACS results in poor communication between various brain areas (i.e., poor brain connectivity). Children affected by FACS usually have smaller brains. In addition, children affected by FACS may develop physical characteristics like microcephaly, growth retardation, dislocated limbs, certain facial features (e.g., thinner upper lip) and cardiological problems. It should be noted that the physiological features of FACS may or may not be present and a percentage of FACS children are misdiagnosed as having ADHD (due to their difficulties with focus, organization, planning, decision making and memories). It should also be noted that Fetal Alcohol Syndrome disproportionally affects babies in the minority communities (specifically in the Black community). No treatment is currently available for FACS.

tPBM (stimulation of the brain with near-infra red light) has been shown in animal and human studies (in vivo and in vitro) to increase blood oxygenation, cerebral blood flow, and mitochondrial ATP production. In addition, EEG and NIRS data has shown that tPBM improves brain connectivity. Therefore, blood brings more oxygen and nutrition to the brain. In addition, increased ATP production results in more neurogenesis and synaptogenesis. Furthermore, functional brain connectivity has been shown to improve after one session. tPBM has been shown to be beneficial for traumatic brain injury, depression, ischemic stroke, and Parkinson's disorder. In addition, it has been shown to be effective for Down syndrome, autism and ADHD. Similarly, tPBM can be effective for the neurological symptoms of FACS by increasing the amount of oxygen and nutrients delivered to the brain, improving functional brain connectivity, and increasing neurogenesis and synaptogenesis. Specifically, the effect may be most pronounced in cortical structures (frontal lobes), which improves organization, focus, and decision making. The effect on memory and motor functions may be less pronounced since sub-cortical structures are implicated (e.g., hippocampus and cerebellum). However, due to neuroplasticity, the beneficial effect of tPBM may be most pronounced when treatment is administered to young children.

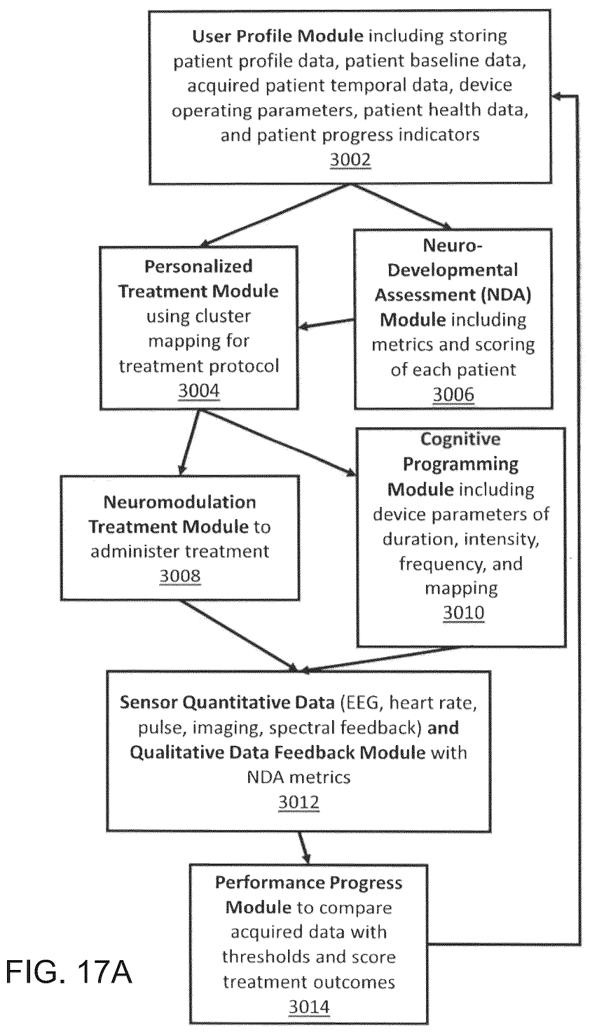

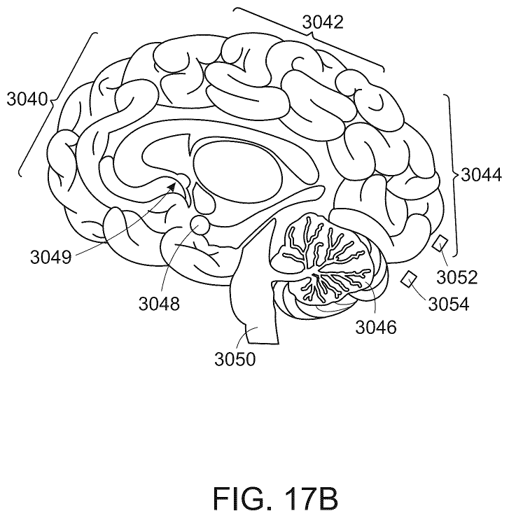

In order to configure the headset, the individual light sources or arrays must be configured to illuminate selected regions of the brain that will address the condition of the patient to be treated. As described previously herein, regions of the brain, as shown in FIG. 17B, can be selected for treatment including the frontal lobes 3040, the parietal lobes 3042, the occipital lobes 3044, the cerebellum 3046 adjacent to the brain stem 3050, the temporal lobes 3049, and the hippocampus 3048. As noted previously, light sources such as LEDs or arrays thereof can be mounted to a headset so as to contact the tissue surface so as to illuminate selected areas. The cerebellum 3046, for example, can be illuminated by LEDs 3052, 3054 positioned adjacent to the occipital lobes and/or the cerebellum so as to treat selected areas. Brain imaging methods can be used to map brain inflammation, for example, that can contribute to the disorder being treated and thereby used to select areas of the brain for treatment.

Neuroinflammation is a response that involves neurons, microglia and macroglia, which are cells that are present in the central nervous system (CNS) (Bradl and Hohlfeld, “Molecular Pathogenesis of Neuroinflammation”, J. Neuro Neurosurg Psychiatry; 74:1364-1370 (2003); Carson et al., 2006a). Neuroinflammation has been reported to characterize many neurodegenerative diseases and neuropsychiatric conditions such as multiple sclerosis, narcolepsy, AD, Parkinson's disease (PD), and ASD (Carson et al., 2006b; Frick et al., 2016). Autistic individuals often show signs of altered inflammatory responses and neuro-immune system abnormalities throughout life, which implicates a potential role of inflammation in the etiology of ASD. This is further confirmed by increasing clinical and experimental evidence that links altered immune and inflammatory responses with the pathogenesis of ASD (Lucchina and Depino, 2014). Moreover, post mortem studies have supported this hypothesis, documenting substantial neuroinflammation in several brain regions of patients with ASD (Vargas et al., 2005).

During pregnancy, both environmental and genetic risk factors may affect inflammatory response of newborns, hence altering postnatal brain development (Adams-Chapman and Stoll, 2006). These genetic and environmental factors can directly elicit chronic neuroinflammation which in turn may modulate neuronal function and immune response via glia activation, or directly by affecting neuronal function (Depino, 2013) (See FIG. 2). Valproic acid (VPA), as an environmental risk factor, elicited activation in different brain regions, with evidence of long-lasting glia activation in the hippocampus and the cerebellum (Lucchina and Depino, 2014). The hippocampus (Depino et al., 2011) and cerebellum (DeLorey et al., 2008; Martin et al., 2010) are two brain regions linked to autism-related behavior, namely, limited social interaction and repetitive behaviors. Additionally, several studies showed that altered social behavior in adult mice may be due to cerebellar inflammation as the cerebellum is considered to be involved in executive and cognitive functions (Shi et al., 2009; Koziol et al., 2014; Lucchina and Depino, 2014; Wang et al., 2014). Furthermore, this evidence suggested that astrocyte and microglia activation in the cortex and cerebellum increase expression of cytokines, including IL-6, TNF-α, MCP-1, TGF-β1, IFN-λ, interferon gamma, IL-8, and other associated genes involved with the immune response in different brain regions of autistic subjects (Vargas et al., 2005; Chez et al., 2007; Garbett et al., 2008; Li et al., 2009; Chez and Guido-Estrada, 2010). Alternatively, both these environmental and genetic factors can chronically alter immune response through increasing production of free radicals, which consequently activate glia cells, increasing the inflammatory response and then affecting neurons, thus mediating clinical symptoms of autism (Depino, 2013). These results suggest that reducing brain inflammation in the targeted brain areas (e.g., prefrontal cortex, cerebellum), can alleviate behavioral symptoms of ASD.

Near infrared light has an anti-inflammatory effect on distressed cells. When such light is used to illuminate oxidatively stressed cells or in animal models of disease, ROS levels are lowered. PBM is able to up-regulate anti-oxidant defenses and reduce oxidative stress. It was shown that PBM can activate NF-κB in normal quiescent cells, however in activated inflammatory cells, inflammatory markers were decreased. One of the most reproducible effects of PBM is an overall reduction in inflammation, which is particularly important for disorders of the joints, traumatic injuries, lung disorders, and in the brain. PBM has been shown to reduce markers of MI phenotype in activated macrophages. Many reports have shown reductions in reactive nitrogen species and prostaglandins in various animal models. PBM can reduce inflammation in the brain, abdominal fat, wounds, lungs, spinal cord.

Children and adolescents with autism often have an enlarged hippocampus. Individuals exhibiting autistic behavior have decreased amounts of brain tissue in parts of the cerebellum.

Default Mode Network (DMN) is under-connected in ASD (Ha et al, 2015). Stimulating nodes of DMN simultaneously increases functional connectivity. Stimulating Cerebellum reduces activation of microglial cells in that region (which is specifically affected by ASD).

The Cluster-Treatment Mapper (CTM) takes the individual's User Profile Model (UPM) vector and maps it into the clusters identified in the Embedded Cluster Predictor (ECP) to identify the optimal treatment options based on the Reference Population Module (RPM). It then feeds the identified cluster into the Personalized Treatment Module (PTM) for further processing.

The entire process for an individual is captured in the flowchart in FIG. 17A. User's data is captured by the UPM, and an neuro-developmental assessment is performed 3006. The PTM leverages the Machine Learning Module (MLM) and the RPM to identify ideal treatments using the Neuromodulation Treatment Module (NMT) and Cognitive Programming Module (CPM) modules. Once the user engages in the treatment, the Sensor and Quantitative Feedback Module SQD module records data on the effect of the treatment, and the Performance Progress Module (PPM) assesses the effectiveness of the treatment, recording all the activities back into the UPM.

Preferred embodiments employ EEG measurements of the cerebellum to characterize improvements in patient function over the course of a series of treatment as described herein. As the cerebellum has been studied extensively in relation to ASD (see D'Mello et al., “Cerebro-cerebellar circuits in autism spectral disorder”, Frontiers in Neuroscience; November 2015, Vol. 9, Art. 408 and van der Heijden et al. “Abnormal Cerebellar Development in Autism Spectrum Disorders”, Dev Neurosci 2021; 43:181-190, the entire contents of each of these references being incorporated herein by reference), the measurement of changes in function by NMR and EEG demonstrate efficacy of the treatment methods as described herein.

Changes in skin color can be addressed in preferred methods described herein by classifying each patient by reference to a known scale of variance in skin pigmentation. See, for example, Everett et al., “Making Sense of Skin Color in Clinical Care”, Clin Nurs Res., 2012 November; 21 (4); 495-516, which employed a spectrophotometer to establish a reference scale with white skin as reference color. The following example illustrates treatment protocols for a white reference color at a selected percentage of maximum light delivery that is used for a darker color of skin at an opposite end of the scale.

Example of changes in light delivery protocol for different skin colors:

### White Skinned Patient:

Session 1:2 minutes at 75% of total power of the device
Session 2:4 minutes at 75% of total power of the device
Session 3:6 minutes at the 75% of total power of the device
Session 4: If hyperactivity is moderate to severe, stay at 6 minutes until session 16. If hyperactivity is minimal and mild then 8 minutes.
Session 5: If hyperactivity is moderate to severe after session 4, then remain at 8 minutes until session 16. If mild hyperactivity, 10 minutes until session 16.
Once full dosage is reached, 12 sessions of treatment are administered.

### Dark Skinned Patient:

Session 1:2 minutes at 100% of total power of the device
Session 2:4 minutes at 100% of total power of the device
Session 3:6 minutes at 100% of total power of the device
Session 4:8 minutes at 100% of total power of the device
Session 5:10 minutes at 100% of total power of the device
Session 6: If strong hyperactivity, stay at 10. If hyperactivity is mild, raise to 12 and stay at 12 until session 18.
Once full dosage is reached, 12 sessions of treatment are administered.

Thus a scale of 1-10 can be employed, for example, in which each number on the scale corresponds to a percentage of 100% of the illuminating power that can be delivered by the device within an established safety limit for which the controller on the headset is programmed.

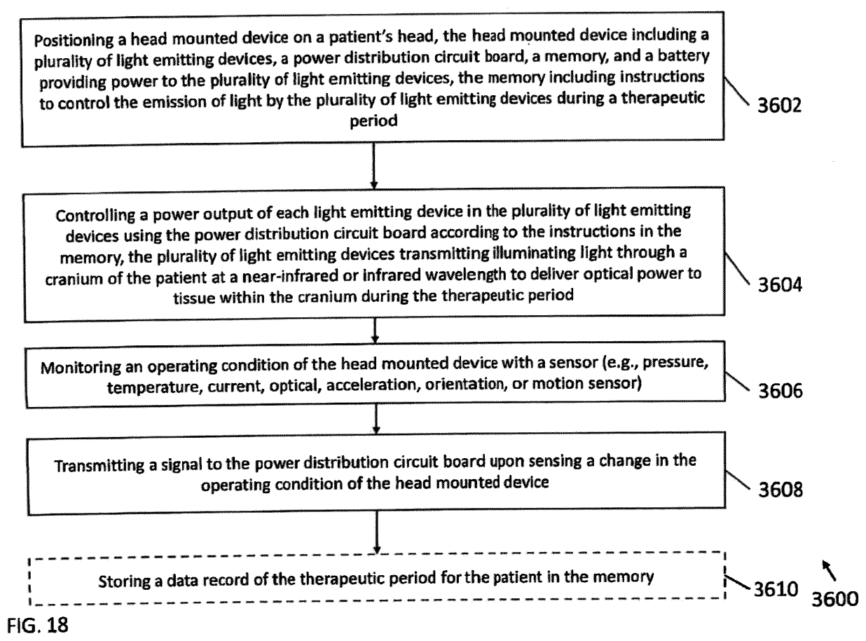

FIG. 18 illustrates a method 3600 for therapeutic photobiomodulation for treatment of diseases or disorders in accordance with some embodiments described herein. The method 3600 includes positioning a head mounted device 5000 on a patient's head (step 3602). The head mounted device 5000 includes a plurality of light emitting devices 5036, a power distribution circuit board 5040, 5050, a memory 5042, and a battery 5060 providing power to the plurality of light emitting devices 5036. The memory 5042 includes instructions to control the emission of light by the plurality of light emitting devices 5036 during a therapeutic period. The method 3600 includes controlling a power output of each light emitting device 5036 in the plurality of light emitting devices using the power distribution circuit board 5040, 5050 according to the instructions in the memory 5042 (step 3604). The plurality of light emitting devices transmits illuminating light through a cranium of the patient at a near-infrared or infrared wavelength to deliver optical power to tissue within the cranium during the therapeutic period. The method 3600 includes a step of monitoring an operating condition of the head mounted device 5000 with a sensor (step 3604). The sensor can be a pressure, temperature, current, optical, or motion sensor in various embodiments. The sensor can measure acceleration or orientation in some embodiments similar to the accelerometer 5045 employed by the patient detection module 5046. Monitoring the operating condition can include detecting adverse events such as monitoring whether the head mounted device 5000 has been removed (advertently or inadvertently) from the patient's head, monitoring temperature to determine if the device is overheating and/or causing the temperature of the tissue being illuminated to exceed a threshold, or monitoring optical power or current to determine whether the device is transmitting too great of an intensity of optical power. The method 3600 can also include transmitting a signal to the power distribution circuit board 5040, 5050 upon sensing a change in the operation condition of the head mounted device 5000 (step 3608). For example, a signal can be sent to the power distribution circuit board 5040, 5050 to stop power upon sensing a parameter that indicates an adverse operating condition. Conversely, a signal can be sent to the power distribution circuit board 5040, 5050 that enables power distribution to the LEDs if the sensor detects that the operating condition is safe (i.e., no errors or warnings). The method 3600 also includes an optional step of storing a data record of the therapeutic period for the patient in the memory 5042 (step 3610). In such an embodiment, the memory 5042 can be non-volatile (e.g., EEPROM or solid-state storage) or the memory 5042 can be volatile memory such as any of the various forms of random access memory (RAM).

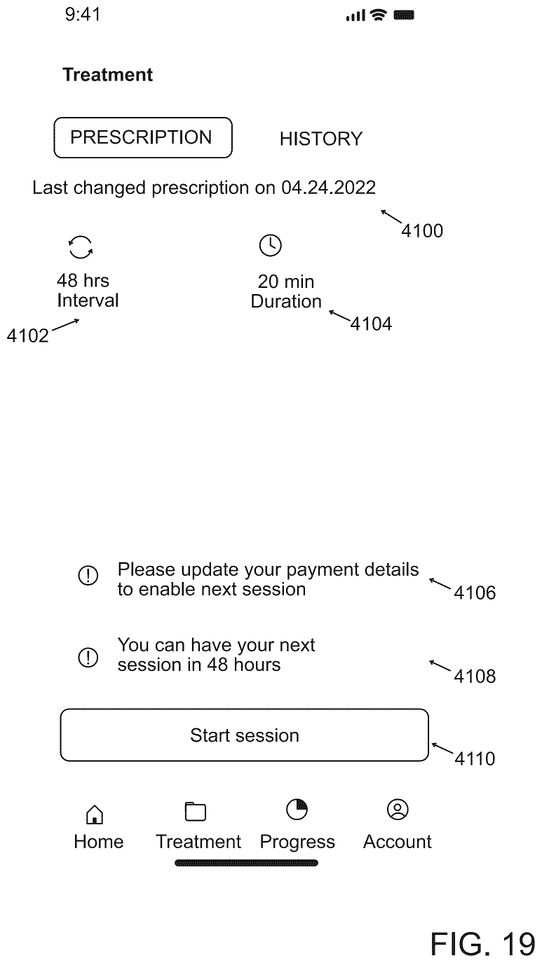

FIG. 19 illustrates a graphical user interface (GUI) 4100 including user prompts to resolve user status restrictions in accordance with various embodiments taught herein. The GUI 4100 can be deployed on an external device as described previously such as the remote computing device 150. The GUI 4100 includes static elements that provide information to the user and actuatable elements that can be actuated by a user to perform certain actions and to control an associated photobiomodulation unit. Actuation of the actuatable elements can occur when the user clicks the element with a mouse, inputs information with a keyboard, or gestures using one or more touches on a touchscreen. Static elements can include prescription information such as prescribed usage interval 4102 and therapeutic program duration 4104. The information displayed in these static elements can be retrieved from a database by the application running the GUI 4100 or can be pushed from a central server (e.g., from a physician's office) to the external device and populated into the appropriate fields in the application.

Actuatable elements can be dynamically displayed in the GUI 4100 when certain conditions are met or can have their appearance modified by the presence of certain conditions. For example, the session start 4110 element can appear on the GUI 4100 in an actuatable state or a deactivated state depending upon whether patient status requirements have been met. If patient status requirements have been met, the session start 4100 element is depicted in an actuatable state that can be actuated by the user to initiate a therapeutic session. If the application determines that patient status requirements have not been met, the session start 4110 element is depicted in a deactivated state that will not respond to user input. The deactivated state can be maintained until the patient status requirements are resolved.

In some embodiments, the use of the photobiomodulation device can be restricted through the application running on the external device until patient status requirements are met. For example, use of the photobiomodulation device can be restricted subject to payment by the patient or user. The application can determine whether the payment requirement of the patient or associated user or associated patient account has been met by, for example, contacting a financial institution or other third-party payment processor and identifying when or whether the most recent payment has been made whether by one-time payment or subscription. The GUI 4100 can prompt the user that the payment requirement has not been met using a payment element 4106 of the GUI 4100. Actuation of the payment element 4106 prompt can take a user to further information or explanation as to what steps should be taken to resolve this patient status requirement. In some embodiments, actuation of the payment element 4106 can take the user to a secure payment portal or front-end site for a financial institution to execute operations that cause a payment to be made using the external device, such as a tablet or mobile phone communication device. These operations can be incorporated into a downloadable software application that includes all operational modules, or any periodic updates thereto, to perform photobiomodulation therapy as generally described herein.

As a further example, use of the photobiomodulation device can be restricted subject to patient usage history. For certain patients it can be necessary to limit the time to sequential therapeutic sessions such as at least 12 hours between sessions, at least 24 hours between sessions or at least 48 hours between sessions. Thus a selected delay time can be prescribed so as to set the light delivery dose over a selected sequence of therapeutic sessions occurring over days, weeks or months. The device can thus employ a programmed calendar that will automatically prevent the device from operating during selected intervals between treatment. In some situations, the prescribed therapeutic regimen may include spaced therapeutic sessions such that the patient is not exposed to photobiomodulation therapy in between the spaced sessions. The application can determine patient usage history either from data stored on the external device (i.e., past sessions that the patient conducted using the external device) or from a database record such as from a doctor's health records. The application can deny or restrict access to usage of the photobiomodulation device until the patient's usage history meets requirements set out in the prescription (e.g., no more than 1, 2, 3, or any specified number of sessions within a set period of time). The GUI 4100 can prompt the user that the patient usage history has not been met using patient history element 4108. Actuation of the patient history element 4108 prompt can take a user to a description of the limitations imposed by the prescription or a countdown clock until the next treatment session can be commenced in accordance with various embodiments.

Additional patient status requirements can include prescription status (e.g., whether a prescription has been issued by a physician, whether a prescription was received from the physician by the external device, whether a prescription has expired) or completion of prerequisites (e.g., usage of the device can be restricted if the user does not complete biographical information input, questionnaires, or surveys before initial treatment or since the most recent treatment session).

Although the static and actuatable elements are shown on GUI 4100 as visual elements, the skilled person would appreciate that other forms for the elements are possible to increase accessibility such as replacing and/or augmenting the visual static or actuatable elements with auditory prompts or prompts to receive speech from the user.

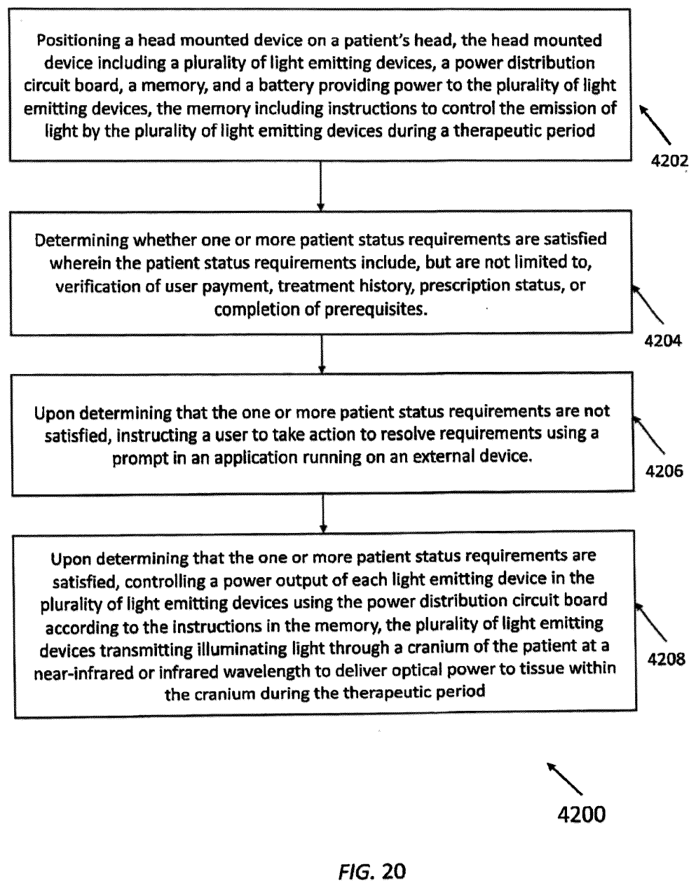

FIG. 20 illustrates a flowchart for a method 4200 for therapeutic photobiomodulation for treatment of diseases or disorders in accordance with various embodiments described herein. The method 4200 includes positioning a head mounted device 5000 on a patient's head (step 4202). The head mounted device 5000 includes a plurality of light emitting devices 5036, a power distribution circuit board 5040, 5050, a memory 5042, and a battery 5060 providing power to the plurality of light emitting devices 5036. The memory 5042 includes instructions to control the emission of light by the plurality of light emitting devices 5036 during a therapeutic period. The method includes determining whether one or more patient status requirements are satisfied wherein the patient status requirements include, but are not limited to, verification of user payment, treatment history, prescription status, or completion of prerequisites (step 4204). The method includes instructing a user to take action to resolve patient status requirements using a prompt in an application running on an external device upon determining that the one or more patient status requirements are not satisfied (step 4206). The method 4200 includes controlling a power output of each light emitting device in the plurality of light emitting devices using the power distribution circuit board according to the instructions in the memory upon determining that the one or more patient status requirements are satisfied (step 4208). The plurality of light emitting devices transmit illuminating light through a cranium of the patient at a near-infrared or infrared wavelength to deliver optical power to tissue within the cranium during the therapeutic period.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

1. A photobiomodulation neuro-therapy device comprising:
a portable head mounted device that is sized to be positioned on a patient's head, the portable head mounted device including a plurality of light emitting devices, a controller, a memory and a battery providing power to the portable head mounted device, each of the light emitting devices being operable in response to control signals to control transmission of transcranial illuminating light into the head of the patient having at least one of a red and a near infrared wavelength delivered during a therapeutic period wherein the processor executes instructions stored in the memory to control the emission of light by the light emitting devices during the therapeutic period, the controller being connected to each of the light emitting devices to control emission of the illuminating light;
wherein each light emitting device is mounted to a panel that is attached to a frame positioned on the patient's head, each panel including a circuit board having a driving circuit to actuate the light emitting device mounted to the panel; and
an adjustable frame on which one or more on or more of the panels are mounted, the frame extending around the head of the patient wherein one or more of the panels is moveable relative to the frame to adjust a position of the light emitting device mounted on each of the one or more panels relative to the frame.

2. The device of claim 1 wherein the controller on a main circuit board mounted to the frame is connected to each of the light emitting devices in parallel to transmit the control signals that separately control emission of light from each of the light emitting devices.

3. The device of claim 1, wherein the frame comprises a plurality of slots wherein one or more of the light emitting devices is positioned to move relative to a corresponding slot between at least two separated positions.

4. The device of claim 1 further comprising a communication circuit including a transceiver on the portable head mounted device to receive a wireless control signal to control an operation of the portable head mounted device.

5. The device of claim 1 wherein an inner surface of the frame is adjustable to conform to a head circumference of the patient.

6. (canceled)

7. (canceled)

8. (canceled)

9. The device of claim 1, wherein the plurality of panels mounted to the frame to illuminate the patient from a plurality of different angles, each panel having one or more light emitting diodes (LEDs)

10. (canceled)

11. The device of claim 1 wherein the controller actuates a wired parallel circuit connected to each light emitting device to independently control operation of each light emitting device.

12. The device of claim 9 wherein each panel in the plurality of panels comprises an LED circuit board on which the at least one LED is mounted, each LED circuit board being connected to a power control circuit mounted on the portable head mounted device.

13. The device of claim 1, wherein the battery is connected to the controller mounted on a main circuit board, the main circuit board being mounted within a housing attached to the frame.

14. The device of claim 4 wherein the communication circuit communicates with the external computing device by a cable, a wireless transmission or a combination thereof.

15. The device of claim 14 wherein the external computing device comprises a tablet display device having a touchscreen display that is operative in response to a plurality of touch gestures made by a user on the surface of the touchscreen display, the tablet display device including a processor programmed with one or more software modules to control operations of the tablet display device and the portable head mounted device.

16. The device of claim 1, wherein a main control circuit operates under closed loop control to control power distribution to each of the light emitting devices.

17. The device of claim 1, wherein an occipital housing is mounted on the frame, the occipital housing further comprising a plurality of panel slots to mount at least one light emitting device in each panel slot, the panel slots comprising openings through an inner panel of the occipital housing.

18. The device of claim 17 wherein the occipital housing includes an outer housing panel such that a circuit board on which the processor, memory and battery are mounted is housed between an inner housing panel and the outer housing panel.

19. The device of claim 1, wherein the controller operates in response to programmed instructions such that the processor executes a sequence of steps to illuminate different regions of brain tissue of the patient with selected levels of light and wherein the selected levels of light are manually selected by a user with a user interface.

20. (canceled)

21. (canceled)

22. The device of claim 1, wherein the portable head mounted device has a size, shape and weight to be worn by a child, the frame being adjustable to size to conform to the size of the child's head and wherein the processor is programmed to treat a neurological condition that comprises autism.

23. The device of claim 1, wherein the portable head mounted device communicates with the external computing device with a wireless connection wherein said communication includes illumination parameters and an illumination period.

24. (canceled)

25. (canceled)

26. The device of claim 9 wherein each LED panel comprises a LED circuit board having an LED extending through an opening in the circuit board, an inner heat transmitting layer and a porous outer panel.

27. The device of claim 9 wherein each LED panel comprises a temperature sensor and an LED indicator circuit and one or more of the LED panels are manually moved along the frame.

28. (canceled)

29. (canceled)

30. The device of claim 1, wherein at least one light emitting device is positioned on the frame to illuminate a temporal lobe of the patient and wherein at least one light emitting device is positioned on the frame or an occipital housing to illuminate an occipital lobe of the patient and optionally wherein at least one light emitting device is positioned on the frame to illuminate a Broca area of the patient and optionally wherein at least one light emitting device is positioned on the frame to illuminate a Wernicke area of the patient.

31. (canceled)

32. (canceled)

33. (canceled)

34. The device of claim 1, wherein at least one light emitting device is positioned on the frame to illuminate the patient's hippocampus and optionally wherein at least one light emitting device is positioned on the frame to illuminate the patient's cerebellum.

35. (canceled)

36. The device of claim 1 further comprising a sensor mounted on the frame or an occipital housing.

37. The device of claim 36 wherein the sensor comprises at least one of a contact sensor to detect contact with the patient's head, and a temperature sensor and an optical sensor.

38. (canceled)

39. (canceled)

40. The device of claim 1 further comprising an EEG electrode mounted on the frame.

41. The device of claim 1, wherein the light emitting devices are driven at a duty cycle having a frequency for a treatment period.

42. The device of claim 1, wherein the portable head mounted device comprises a plurality of light emitting devices including at least six light emitting diodes (LEDs) LEDs mounted at separate positions on the frame to illuminate different regions of the patient's brain.

43. The device of claim 1, wherein a main control circuit operates the light emitting devices at a substantially constant current and further comprising a patient detection circuit connected to a contact sensor, the patient detection circuit connected to a switch to shut off the light emitting devices upon removal of the portable head mounted device from the patient's head.

44. (canceled)

45. A photobiomodulation neuro-therapy device comprising:
a portable head mounted device that is sized to be positioned on a patient's head, the portable head mounted device including a plurality of light emitting devices, a control circuit, a memory and a battery providing power to the portable head mounted device, each of the light emitting devices being operable in response to control signals to control transmission of transcranial illuminating light into the head of the patient having at least one of a red and a near infrared wavelength delivered during a therapeutic period wherein the control circuit processor executes instructions stored in the memory to control the emission of light by the light emitting devices during the therapeutic period; and
a frame on the portable head mounted device to adjust a position of one or more of the plurality of light emitting devices relative to the frame wherein one or more of the light emitting devices are configured to move between at least two separated positions on the frame for emission of transcranial illumination.

46. The device of claim 45 wherein the control circuit is connected to each of the light emitting devices in parallel to transmit the control signals that separately control emission of light from each of the light emitting devices.

47. The device of claim 45, wherein the frame comprises a plurality of slots wherein one or more of the light emitting devices is positioned to move relative to a corresponding slot between the at least two separated positions.

48. The device of claim 45 further comprising a communication circuit including a transceiver on the portable head mounted device to receive a wireless control signal to control an operation of the portable head mounted device.

49. The device of claim 45 wherein an inner surface of the frame is adjustable to conform to a head circumference of the patient.

50. (canceled)

51. (canceled)

52. (canceled)

53. The device of claim 45 wherein the light emitting device further comprises a plurality of panels mounted to the frame to illuminate the patient from a plurality of different angles, each panel having one or more light emitting diodes (LEDs) and wherein each panel in the plurality of panels comprises an LED circuit board on which the at least one LED is mounted, each LED circuit board being connected to a power control circuit mounted on the portable head mounted device.

54. (canceled)

55. The device of claim 45 wherein the control circuit comprises a wired parallel circuit connected to each light emitting device to independently control operation of each light emitting device.

56. (canceled)

57. The device of claim 45, wherein the battery is connected to the control circuit mounted on a controller circuit board, the controller circuit board is mounted within a housing attached to the frame and wherein the communication circuit communicates with an external computing device by a cable, a wireless transmission or a combination thereof.

58. (canceled)

59. The device of claim 57, wherein the external computing device comprises a tablet display device having a touchscreen display that is operative in response to a plurality of touch gestures made by a user on the surface of the touchscreen display, the tablet display device including a processor programmed with one or more software modules to control operations of the tablet display device and the portable head mounted device.

60. The device of claim 45, wherein the control circuit operates under closed loop control to control power distribution to each of the light emitting devices.

61. The device of claim 45, wherein an occipital housing is mounted on the frame, the occipital housing further comprising a plurality of panel slots to mount at least one light emitting device in each panel slot, the panel slots comprising openings through an inner panel of the occipital housing.

62. The device of claim 61 wherein the occipital housing includes an outer housing panel such that a circuit board on which the processor, memory and battery are mounted is housed between an inner housing panel and the outer housing panel.

63. The device of claim 45, wherein the control circuit operates in response to programmed instructions such that the processor executes a sequence of steps to illuminate different regions of brain tissue of the patient with selected levels of light and wherein the selected levels of light are manually selected by a user with a user interface.

64. (canceled)

65. (canceled)

66. The device of claim 45, wherein the portable head mounted device has a size, shape and weight to be worn by a child, and wherein the processor is programmed to treat a neurological condition that comprises autism.

67. The device of claim 45, wherein the portable head mounted device communicates with the external computing device with a wireless connection wherein said communication includes illumination parameters and an illumination period, the external computing device having a user interface comprising a graphical user interface operable on a tablet touchscreen display.

68. (canceled)

69. (canceled)

70. The device of claim 53 wherein each LED panel comprises a circuit board having an LED extending through an opening in the circuit board, an inner heat transmitting layer and a porous outer panel.

71. The device of claim 53 wherein each LED panel comprises a temperature sensor and an LED indicator circuit and one or more LED panels move along the frame.

72. (canceled)

73. (canceled)

74. The device of claim 45, wherein at least one light emitting device is positioned on the frame to illuminate a temporal lobe of the patient or positioned on the frame or an occipital housing to illuminate an occipital lobe of the patient, or optionally positioned on the frame to illuminate a Broca area of the patient, or optionally positioned on the frame to illuminate a Wernicke area of the patient, or optionally positioned on the frame to illuminate the patient's hippocampus or positioned on the frame to illuminate the patient's cerebellum.

75-79. (canceled)

80. The device of any one of claims 45-79 further comprising a sensor mounted on the frame or an occipital housing wherein the sensor optionally comprises a contact sensor to detect contact with the patient's head, or wherein the sensor comprises a temperature sensor, or wherein the sensor comprises an optical sensor.

81-83. (canceled)

84. The device of claim 45 further comprising an EEG electrode mounted on the frame.

85. The device of claim 45, wherein the light emitting devices are driven at a duty cycle having a frequency for a treatment period and wherein the control circuit operates with at least a 400 milliamp substantially constant current to actuate the light emitting devices at a duty cycle of less than 45%.

86. (canceled)

87-127. (canceled)
